SEQUENCE LISTING (157 sequences)

SEQ ID NO: 1 - DNA, 3206 bp, Artificial Sequence
  Codon optimized sequence for translation into mammalian cells
ggcgcgccaagcttgcggccgcggtaccgctagcgccgccaccatggccgccagcttcccccctaccctgggcctgagcagcgcccctgacgagatccagcacccccacatcaagttcagcgagtggaagttcaagctgttcagagtgcggagcttcgaaaagacccccgaggaggcccagaaggagaagaaggacagcttcgagggcaagcccagcctggagcagagccctgccgtgctggacaaggccgacggccagaagcccgtccccacccagcccctgctgaaggcccaccccaagttcagcaagaagttccacgacaacgagaaggccaggggcaaggccatccaccaggccaacctgcggcacctgtgccggatctgcggcaacagcttccgggccgacgagcacaaccggcgctaccccgtgcacggccccgtggacggcaagaccctgggactgctgcggaagaaggagaagcgggccacctcctggcccgacctgatcgccaaggtgttcaggatcgacgtgaaggccgacgtggacagcatccaccccaccgagttctgccacaactgctggtccatcatgcaccggaagttcagctccgccccctgcgaggtgtacttcccccggaacgtgaccatggagtggcaccctcacacccccagctgcgacatctgcaacaccgccagacggggcctgaagcggaagtccctgcagcccaacctgcagctgtccaagaagctgaaaaccgtcctggatcaggcccggcaggccaggcagagaaagcggagagcccaggcccggatcagcagcaaggacgtgatgaagaagatcgccaactgcagcaagatccacctgagcaccaagctgctggccgtggacttccccgagcacttcgtgaagtccatcagctgccagatctgcgagcacatcctggccgaccccgtggagacaaactgcaagcacgtgttctgcagagtgtgcatcctgcggtgcctgaaggtgatgggcagctactgccccagctgcagatacccctgcttccccaccgacctggagagccccgtgaagtccttcctgagcgtgctgaacagcctgatggtgaagtgccccgccaaggagtgcaacgaggaggtctccctggagaagtacaaccaccacatcagcagccacaaggagagcaaggagatcttcgtccacatcaacaagggcggcagaccccggcagcacctgctgtccctgaccagacgggcccagaagcacagactgcgggagctgaagctgcaggtgaaggccttcgccgacaaggaggagggcggcgacgtcaagtccgtgtgcatgaccctgtttctgctggccctgagagctaggaacgagcaccggcaggccgatgagctggaggccatcatgcagggcaagggcagcggcctgcagcctgccgtgtgcctggccatcagagtgaacacctttctgagctgcagccagtaccacaagatgtaccggaccgtgaaggccatcaccggcagacagatcttccagcctctgcacgccctgcggaacgccgagaaggtgctgctgcccggctaccaccacttcgagtggcagccccccctgaagaacgtgtccagcagcaccgacgtgggcatcatcgacggcctgagcggcctgtccagctccgtggacgactaccctgtggacacaatcgccaagcggttcagatacgacagcgccctggtgtccgccctgatggacatggaggaggacatcctggagggcatgcggagccaggacctggacgattacctgaacggccccttcaccgtggtggtgaaagaatcctgcgacggcatgggcgacgtgtccgagaagcacggcagcggccctgtggtgcccgagaaggccgtgcggttcagcttcaccatcatgaagatcacaatcgcccacagcagccagaacgtgaaggtgttcgaggaggccaagcccaacagcgagctgtgctgcaagcccctgtgcctgatgctggccgacgagagcgaccacgagacactgaccgccatcctgagccccctgatcgccgagcgggaggccatgaagtcctccgagctgatgctggagctgggcggcatcctgaggaccttcaagttcatcttccggggcaccggctacgacgagaagctggtgcgggaggtggagggcctggaggccagcggcagcgtgtacatctgcaccctgtgcgacgccacccggctggaggcctcccagaacctggtgttccacagcatcacccggtcccacgccgagaacctggagagatacgaggtgtggcggagcaacccctaccacgagagcgtggaggagctgcgggacagagtgaagggcgtgagcgccaagcccttcatcgagacagtgcccagcatcgacgccctgcactgcgatatcggcaacgccgccgagttctacaagatctttcagctggagatcggagaggtgtacaagaaccccaacgccagcaaggaggagcggaagcgctggcaggccaccctggacaagcacctgcgcaagaagatgaacctgaagcccatcatgcggatgaacggcaacttcgccagaaagctgatgaccaaggaaacagtggacgccgtctgcgagctgatccccagcgaggagcggcacgaggccctgcgcgagctgatggacctgtacctgaagatgaagcccgtgtggcggtccagctgtcctgccaaggagtgtcccgagagcctgtgccagtacagcttcaacagccagcggttcgccgagctgctgtccaccaagttcaagtaccgctacgagggcaagatcaccaactacttccacaagacactggcccacgtgcccgagatcatcgagcgggacggcagcatcggcgcctgggccagcgagggcaacgagagcggcaacaagctgttccggcggttcaggaagatgaacgccaggcagagcaagtgctacgagatggaggacgtgctgaagcaccactggctgtacaccagcaagtacctgcagaaattcatgaacgcccacaacgccctgaaaaccagcggcttcaccatgaaccctcaggccagcctgggcgaccctctgggcatcgaggactccctggagtcccaggacagcatggaattctgataatctagagcggccgcggatccttaattaa

SEQ ID NO: 2 - protein, 1043 aa, Homo sapiens
MAASFPPTLGLSSAPDEIQHPHIKFSEWKFKLFRVRSFEKTPEEAQKEKKDSFEGKPSLEQSPAVLDKADGQKPVPTQPLLKAHPKFSKKFHDNEKARGKAIHQANLRHLCRICGNSFRADEHNRRYPVHGPVDGKTLGLLRKKEKRATSWPDLIAKVFRIDVKADVDSIHPTEFCHNCWSIMHRKFSSAPCEVYFPRNVTMEWHPHTPSCDICNTARRGLKRKSLQPNLQLSKKLKTVLDQARQARQRKRRAQARISSKDVMKKIANCSKIHLSTKLLAVDFPEHFVKSISCQICEHILADPVETNCKHVFCRVCILRCLKVMGSYCPSCRYPCFPTDLESPVKSFLSVLNSLMVKCPAKECNEEVSLEKYNHHISSHKESKEIFVHINKGGRPRQHLLSLTRRAQKHRLRELKLQVKAFADKEEGGDVKSVCMTLFLLALRARNEHRQADELEAIMQGKGSGLQPAVCLAIRVNTFLSCSQYHKMYRTVKAITGRQIFQPLHALRNAEKVLLPGYHHFEWQPPLKNVSSSTDVGIIDGLSGLSSSVDDYPVDTIAKRFRYDSALVSALMDMEEDILEGMRSQDLDDYLNGPFTVVVKESCDGMGDVSEKHGSGPVVPEKAVRFSFTIMKITIAHSSQNVKVFEEAKPNSELCCKPLCLMLADESDHETLTAILSPLIAEREAMKSSELMLELGGILRTFKFIFRGTGYDEKLVREVEGLEASGSVYICTLCDATRLEASQNLVFHSITRSHAENLERYEVWRSNPYHESVEELRDRVKGVSAKPFIETVPSIDALHCDIGNAAEFYKIFQLEIGEVYKNPNASKEERKRWQATLDKHLRKKMNLKPIMRMNGNFARKLMTKETVDAVCELIPSEERHEALRELMDLYLKMKPVWRSSCPAKECPESLCQYSFNSQRFAELLSTKFKYRYEGKITNYFHKTLAHVPEIIERDGSIGAWASEGNESGNKLFRRFRKMNARQSKCYEMEDVLKHHWLYTSKYLQKFMNAHNALKTSGFTMNPQASLGDPLGIEDSLESQDSMEF

SEQ ID NO: 3 - DNA, 1661 bp, Artificial Sequence
  Codon optimized sequence for translation into mammalian cells
ggcgcgccgaattcgcggccgcggtaccgctagcaagcttgccgccaccatgagcctgcagatggtgaccgtgtccaacaatatcgccctgatccagcccggcttcagcctgatgaacttcgacggccaggtgttcttcttcggccagaagggctggcccaagcggagctgccccaccggcgtgttccacctggacgtgaagcacaaccacgtgaagctgaagcctaccatcttcagcaaggacagctgctacctgccccccctgcgctaccctgccacctgcaccttcaagggcagcctggagagcgagaagcaccagtacatcatccacggcggcaagacacccaacaacgaggtgtccgacaagatctacgtgatgagcatcgtgtgcaagaacaacaagaaggtgaccttccgctgcaccgagaaggacctggtgggagatgtgcccgaggccagatacggccactccatcaacgtggtgtacagccggggcaagagcatgggcgtgctgttcggcggcaggtcctacatgcccagcacccaccggaccaccgagaagtggaacagcgtggccgactgcctgccctgcgtgttcctggtggacttcgagttcggctgcgccacctcctacatcctgccagagctgcaggacggcctgtccttccacgtgtctatcgccaagaacgacaccatctacatcctgggcggccacagcctggccaacaacatcaggcccgccaacctgtaccggatcagggtggacctgcccctgggcagcccagccgtgaactgcaccgtgctgcctggcggcatcagcgtgtcctctgccatcctgacccagaccaacaacgacgagttcgtgatcgtgggcggctaccagctggagaaccagaaacggatgatctgcaacatcatcagcctggaggacaacaagatcgagatccgggagatggagacacccgactggacccctgacatcaagcacagcaagatctggttcggcagcaacatgggcaacggcaccgtgtttctgggcatccccggcgacaacaagcaggtggtgtccgagggcttctacttctacatgctgaagtgcgccgaggacgacaccaacgaggagcagaccaccttcaccaacagccagaccagcaccgaggaccccggcgactccacccccttcgaggacagcgaggagttttgcttcagcgccgaggccaacagcttcgacggcgacgacgagtttgacacctacaacgaggacgacgaggaggacgagtccgagacaggctactggatcacctgctgccctacctgcgacgtggatatcaacacctgggtgcccttctacagcaccgagctgaacaagcccgccatgatctactgcagccacggcgacggccactgggtgcacgcccagtgcatggacctggccgagcggaccctgatccacctgtccgccggctccaacaagtactactgcaacgagcacgtggagatcgccagggccctgcacaccccccagagagtgctgcctctgaaaaagccccctatgaagtccctgaggaagaagggctccggcaagatcctgacccccgccaagaagtcctttctgcggcggctgttcgactgagcggccgctctagactcgagttaattaa

SEQ ID NO: 4 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 5 - DNA, 1551 bp, Artificial Sequence
  Codon optimized sequence for translation into mammalian cells
aagcttgccgccaccatggacccccccagagccagccacctgagccccagaaagaagagacccagacagaccggcgccctgatggccagcagcccccaggacatcaagttccaggacctggtggtgttcatcctggagaagaagatgggcaccaccagaagagccttcctgatggagctggccagaagaaagggcttcagagtggagaacgagctgagcgacagcgtgacccacatcgtggccgagaacaacagcggcagcgacgtgctcgagtggctgcaggcccagaaggtgcaggtgagcagccagcccgagctgctggacgtgagctggctgatcgagtgcatcagagccggcaagcccgtggagatgaccggcaagcaccagctggtggtgagaagagactacagcgacagcaccaaccccggcccccccaagaccccccccatcgccgtgcagaagatcagccagtacgcctgccagagaagaaccaccctgaacaactgcaaccagattttcaccgacgccttcgacatcctggccgagaactgcgagttcagagagaacgaggacagctgcgtgaccttcatgagagccgccagcgtgctgaagagcctgcccttcaccatcatcagcatgaaggacaccgagggcatcccctgcctgggcagcaaggtgaagggcatcatcgaggagatcatcgaggacggcgagagcagcgaggtgaaggccgtgctgaacgacgagagataccagagcttcaagctgttcaccagcgtgttcggcgtgggcctgaagaccagcgagaagtggttcagaatgggcttcagaaccctgagcaaggtgagaagcgacaagagccttaagttcaccagaatgcagaaggccggcttcctgtactacgaagatctggtgagctgcgtgaccagagccgaggccgaggccgtgagcgtgctggtgaaggaggccgtgtgggccttcctgcccgacgccttcgtgaccatgaccggcggcttcagaagaggcaagaagatgggccacgacgtggacttcctgatcaccagccccggcagcaccgaggacgaggagcagctgctgcagaaggtgatgaacctgtgggagaagaagggcctgctgctgtactacgacctggtggagagcaccttcgagaagctgagactgcccagcagaaaggtggacgccctggaccacttccagaagtgcttcctgatcttcaagctgcccagacagagagtggacagcgaccagagcagctggcaggagggcaagacctggaaggccatcagagtggacctggtgctgtgcccctacgagagaagagccttcgccctgctgggctggaccggcagcagacagttcgagagagacctgagaagatacgccacccacgagagaaagatgatcctggacaaccacgccctgtacgacaagaccaagagaatcttcctgaaggccgagagcgaggaggaaatcttcgcccacctgggcctggactacatcgagccctgggagagaaacgcctgatctaga

SEQ ID NO: 6 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 7 - DNA, 3385 bp, Artificial Sequence
  LacZ recombination signal sequence
ttaattaagcttctgcacctcgaagggtacctactgtgcgagagacacagtgctccagggctgaacaaaaaccgaattctcacttctggcaccacaccagctgatagtggtatctgccggcggacagctgaaactcggcggacacgctgggggaccagctgtcgtcgccgccgatgcccatgtggaagccgtcgatgttcagccaggtgccctcctcggcgtgcagcaggtgccggtggcttgtctccatcagctgctgctgagagtaccggctgatgttgaactggaagtcgcccctccactggtgggggccgtagttcagctcccgggtgccgcatctcaggccattctcgctggggaacacgtagggggtgtacatgtcgctcagaggcaggtcccatctgtcgaagcaggcggcggtcagccggtcggggtagttctcctgggggcccaggcccagccagttcactctctcggccacctgggccagctgacagttcaggccgattctggcggggtgaggggtgtcggaggccacctccacgtccactgtgatggccatctggccgctgccgtcgatccggtaggtctttctggagatgaacagtgtcttgccctggtgctgccaagcgtgggcggtggtgatcagcacggcgtcggccagggtatcggcggtgcactgcagcagggcggcctcggcctggtagtgtccggcagccttccaccgctccacccaggcgttggggtcgatccgggtggcctcgctcacgccgatgtcgttgtccaggggggctctggtgaactggtcccgcaggggggtcagcagctgcttcttgtcgccgatccacatctgggacaggaagccgctctgccggttgaactgccaccgcttgttgcccagctcgatgcaaaagtccatctcggaggtggtcaggtgggggatggcgtggctggcggcaggcagggtcacggacaggttctcggccagccgccactgctgccaggcgctgatgtggccggcctcggaccaggcggtggcgttgggctgcaccacccgcacggtcagccacagctggccggcagactcaggctggggcagctcaggcagctcgatcagctgcttgccctggggggccacgtccagtggcacctcgccggaggccagaggcttgccgtccagggccaccatccagtgcagcagctcgttgtcgctgtgccggaacaggtactcgctggtcacctcgatggtctggccggacagccggaactggaagaactgctgctggtgcttggcttcggtcagggcagggtggggggtccggtcggcgaacaccaggccattcatgcagaactgccggtcattgggggtgtcgccgaagtcgccgccgtaggcgctccaagggttgccgttctcgtcgtacttgatcaggctctggtccacccagtcccacacaaagccgccctgcagccgggggtactgccggaaggcctgccagtacttggcgaagccgcccaggctgttgcccatggcgtgggcgtactcgcacaggatcaggggccgtgtctcgccgggcagggacagccacttcttgatgctccacttaggcacggcggggaagggctggtcctcgtccactctggcgtacatggggcagatgatgtcggtggcggtggtgtcggctcctccgccctcgtactgcacgggcctgctggggtccacgctcttgatccaccggtacagggcgtcgtggttggcgccgtggccgctctcgttgcccagggaccagatgatcacgctggggtggttccggtcccgctgcaccatccgggtcacgcgctcgctcatggcgggcagccatctggggtcgtcggtcagcctgttcatgggcaccatgccgtgtgtctcgatgttggcctcgtccaccacgtacaggccgtatctgtcgcacagggtgtaccacagagggtggttggggtagtggctgcaccgcacggcgttgaagttgttctgcttcatcagcaggatgtcctgcaccatggtctgctcgtccatcacctggccgtgcagggggtggtgctcgtgccggttcacgccccggatcagcaggggcttgccgttcagcagcagcaggccgttctcgatccgcacctcccggaagcccacgtcgcaggcctcggcctcgatcagggtgccgtcggcggtgtgcagctccaccacggcccggtacaggttggggatctcggcgctccacagcttggggttctccacgttcagccgcagggtcactctgtcggcgtagccgcccctctcgtcgatgatctcgccgccgaaaggggcggtgccgctggccacctgtgtctcgccctgccacagggacacggtcactctcaggtagtcccgcagctcgccgcacatctgcacctcggcctccagcacggccctgctgaagtcgtcgttgaaccgggtggccacgtggaagtcgctgatctgggtggtgggcttgtgcagcagggacacgtcccggaagatgccgctcatccgccacatgtcctggtcctccaggtagctgccgtcgctccaccgcagcaccatcacggccagcctgttctcgccggctctcaggaaggcgctcaggtcgaactcgctgggcagccggctatcctggccgtagcccacccatctgccgttgcaccacaggtggaaggcgctgttcacgccgtcgaagatgatcctggtctggccctcctgcagccaggactcgtccacgttgaaggtcaggctgtagcagccggtggggttctcggtgggcacgaagggggggttcacggtgatggggtaggtcacgttggtgtagatgggggcgtcgtagccgtgcatctgccagttgctgggcaccaccacggtgtcggcctcgggcaggtcgcactccagccagctctcgggcacggcctcgggggcagggaaccaggcgaaccgccactcgccgttcaggctccgcagctgctgggagggcctgtcggtccgggcctcctcgctgttccgccagctggcaaagggagggtgggcggccagccggttcagctgggtcacgccagggttctcccagtcccgccgctgcagcaccacggccaggctgtcggtgatcatggtggcggctctagactagatcttccggaacacactggcctcccacagtggtagtactccactgtctgggtgtacaaaaaccggatcctttaccagacatgataagatacattgatgagtttggacaaaccacaactagaatgcagtgaaaaaaatgctttatttgtgaaatttgtgatgctattgctttatttgtaaccattataagctgcaataaacaagttctcgaggcgcgcc

SEQ ID NO: 8 - protein, 1022 aa, Artificial Sequence
  LacZ recombination signal sequence
MITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 9 - DNA, 889 bp, Artificial Sequence
  Gene optimized sequence for expression in mammalian cells
ggcgcgccaagcttgccgccaccatggacatgcgggtgcccgcccagctcctggggctcctgctactctggctccgaggtaaggatggagaacactaggaatttactcctcgagctcgcggccgcagccagtgtgctcagtactgactggaacttcagggaagttctctgataacatgattaatagtaagaatatttgtttttatgtttccaatctcaggtgccagatgtgacatccagatgacccagagccccagcagcctgagcgccagcgtgggcgacagagtgaccatcacctgccgggccagccagagcatcagcaactacctgaactggtatcagcagaagcccggcaaggcccccaagttcctgatctacggcgccagctccctggaaagcggcgtgcccagccggtttagcggcagcggctccggcaccgacttcaccctgaccatcagcagcctgcagcccgaggacttcgccacctactactgccagcagagctacagcaaccccctgacctttggcggcggaacaaaggtggagatcaagcggaccgtggccgctcccagcgtgttcatcttcccccccagcgacgagcagcttaagagcggtaccgctagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgctaatctagattaattaa

SEQ ID NO: 10 - protein, 236 aa, Artificial Sequence
  Gene optimized sequence for expression in mammalian cells
MDMRVPAQLLGLLLLWLRGARCDIQMTQSPSSLSASVGDRVTITCRASQSISNYLNWYQQKPGKAPKFLIYGASSLESGVPSRFSGSGSGTDFTLTISSLQPEDFATYYCQQSYSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 3134 bp, Artificial Sequence
  A heavy chain vector designed to express human IgG on the cell surface
ggcgcgccggatccactagccagtgtggtgcttaagtgcagatatcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagcggccgcgccgccaccatggggtcaaccgccatcctcgccctcctcctggctgttctccaaggagtctgtgccgaggtgcagctggtgcagtctggagcagaggtgaaaaagcccggggagtctctgaaaatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctccagtggagcagcctgaaggcctcggacaccgccatgtattactgtgcgagacaggacggcgacagctttgactactggggccagggaaccctggtcaccgtctcctcaggtgagtcctcacaacctctctcctgctttaactctgaagggttttgctgcatttctggggggaaataagggtgctgggtctcctgccaagagagcccctgcagagggccaccctaggcctctggggtccaatgcccaacaacccccgggccctccccgggctcagtctgagagggtcccagggacgtagcggggcgccggtttttgtacacccagacagtggagtactaccactgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaggaggccagcagagggttccatgagaagggcaggacagggccacggacagtcagcttccatgtgacgcccggagacagaaggtctctgggtggctggtttttgtacacccagacagtggagtactaccactgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggacgctagcgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcataccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgaataacttcgtataatgtatgctatacgaagttatgggccacatggaattcagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggcaaagctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgacgcgt

SEQ ID NO: 12 - protein, 515 aa, Artificial Sequence
  A heavy chain vector designed to express human IgG on the cell surface
MGSTAILALLLAVLQGVCAEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQDGDSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKAVGQDTQEVIVVPHSLPFKVVVISAILALVVLTIISLIILIMLWQKKPR

SEQ ID NO: 13 - DNA, 11 bp, Artificial Sequence
  D segment encoding sequence
ctaactgggga

SEQ ID NO: 14 - DNA, 4193 bp, Artificial Sequence
  Nucleotide sequence variant of the V64 antibody generation vector
ggatccactagccagtgtggtgcttaagtgcagatatcgcggccgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggggtcaaccgccatcctcgccctcctcctggctgttctccaaggagtctgtgccgaggtgcagctggtgcagtctggagcagaggtgaaaaagcccggggagtctctgaaaatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctccagtggagcagcctgaaggcctcggacaccgccatgtattactgtgcgagacacacagtggtagtactccactgtctgggtgtacaaaaacctccacaccgcaggtgcagaaactagtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggagtttgggctgagctgggttttccttgttgctattataaaaggtgtccagtgtcaggtacagctccagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctccagctgaactctgtgactcccgaggacacggctgtgtattactgtgcaagagacacagtggtagtactccactgtctgggtgtacaaaaacctccctgcacggatgctcaggtccggaccagtgggcaccctcttccaggacagtcctcagtgatatcacatcgggaacccacatctggatcaggacggcacccagaacacaagatggcccatggggacagccccacagcccagcccttcccagacccctaaaaggtgtcccaccccctgcacctaccccaggactaaaaatccaggaggcctgactcctgcacatgctctgaccggatgtcacctcggcccctcctggaggggacaggagccctggagggtgagtcagaccctcctgccctcgacggcaggcggggaagattcagaccggtctgagatccccaggatgcagcaccactgtcaatgggggccccagacgcctggaccagcacctgcgtgggaaatgcctctgggctcactgaggggctttttgtgaaggccctcctgctatgtgactatggtgctaactaccacagtgatgaacccagcagcaaaaactgaccggactcccagggtttatgcacacttctcggctcagagttctccaggataagaagagccaggcccaaggatttctgcccagaccctcggcctctagggacaccttggccatgaaagcccatgggctggtgccccacacttcatctgccttcaaacaagggcttcagagggctctgaggtgacctcactcatgaccacaggtgcctgccagctgcaccgaaccctgtcccaacagctgccacagttccaacagccaattcctagggccgggaattgctgtagacaccagccttgttccagcacctcctgccaattgcctggattcccatcctggctggaatcaagagggcagcatccgcaagcttatgctcccccgggaccccgggctgtggtttttgtacacccagacagtggagtactaccactgtggctgaatacttccagcactggggccagggcaccctggtcaccgtctcctcaggtgagtctgctgtctggggatagcggggagccaggtgtactgggccaggcaagggctttgggctccttctccggctgtttgggaccacgttcagcagaaggcctttctttgggaactgggactctgctgctggggggcttcagacttggggacaggtgctcagcaaaggaggtcggcaggagggcggagggtggtttttgtacacccagacagtggagtactaccactgtgctactggtacttcgatctctggggccgtggcaccctggtcactgtctcctcaggtgagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggcaaagggtgggcagagtcatgcttgtgctggggacaaaatgaccttgggacacggggctggctgccacggccggcccgggacagtcggagagtcaggtttttgtacacccagacagtggagtactaccactgtgactactttgactactggggccagggaaccctggtcaccgtctcctcaggtgagtcctcacaacctctctcctgctttaactctgaagggttttgctgcatttctggggggaaataagggtgctgggtctcctgccaagagagcccctgcagagggccaccctaggcctctggggtccaatgcccaacaacccccgggccctccccgggctcagtctgagagggtcccagggacgtagcggggcgccggtttttgtacacccagacagtggagtactaccactgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaggaggccagcagagggttccatgagaagggcaggacagggccacggacagtcagcttccatgtgacgcccggagacagaaggtctctgggtggctggtttttgtacacccagacagtggagtactaccactgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggacgctagc

SEQ ID NO: 15 - DNA, 4205 bp, Artificial Sequence
  Nucleotide sequence variant of the V64 antibody generation vector
ggatccactagccagtgtggtgcttaagtgcagatatcgcggccgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggggtcaaccgccatcctcgccctcctcctggctgttctccaaggagtctgtgccgaggtgcagctggtgcagtctggagcagaggtgaaaaagcccggggagtctctgaaaatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctccagtggagcagcctgaaggcctcggacaccgccatgtattactgtgcgagacacacagtggtagtactccactgtctgggtgtacaaaaacctccacaccgcaggtgcagaaactagtctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggagtttgggctgagctgggttttccttgttgctattataaaaggtgtccagtgtcaggtacagctccagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctccagctgaactctgtgactcccgaggacacggctgtgtattactgtgcaagagacacagtggtagtactccactgtctgggtgtacaaaaacctccctgcacggatgctcaggtccggaccagtgggcaccttcttccaggacattcctcggtcgcatcacagcaggcacccacatctggatcaggacggcccccagaacacaagatggcccatggggacagccccacaacccaggccttcccagacccctaaaaggcgtcccaccccctgcacctgccccagggctaaaaatccaggaggcttgactcccgcataccctccagccagacatcacctcagccccctcctggaggggacaggagcccgggagggtgagtcagacccacctgccctcgatggcaggcggggaagattcagaaaggcctgagatccccaggacgcagcaccactgtcaatgggggccccagacgcctggaccagggcctgcgtgggaaaggccgctgggcacactcaggggctttttgtgaaggcccctcctactgtgtgactacggtgactaccacagtgatgaaactagcagcaaaaactggccggacacccagggaccatgcacacttctcagcttggagctctccaggaccagaagagtcaggtctgagggtttgtagccagaccctcggcctctagggacaccctggccatcacagcagatgggctggtgccccacatgccatctgctccaaacaggggcttcagagggctctgaggtgacttcactcatgaccacaggtgccctggccccttccccgccagctacaccgaaccctgtcccaacagctgccccagttccaacagccaattcctggggcccagaattgctgtagacaccagcctcgttccagcacctcctgccaattgcctggattcacatcctggctggaatcaagagggcagcatccgcaagcttatgctcccccgggaccccgggctgtggtttttgtacacccagacagtggagtactaccactgtggctgaatacttccagcactggggccagggcaccctggtcaccgtctcctcaggtgagtctgctgtctggggatagcggggagccaggtgtactgggccaggcaagggctttgggctccttctccggctgtttgggaccacgttcagcagaaggcctttctttgggaactgggactctgctgctggggggcttcagacttggggacaggtgctcagcaaaggaggtcggcaggagggcggagggtggtttttgtacacccagacagtggagtactaccactgtgctactggtacttcgatctctggggccgtggcaccctggtcactgtctcctcaggtgagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggcaaagggtgggcagagtcatgcttgtgctggggacaaaatgaccttgggacacggggctggctgccacggccggcccgggacagtcggagagtcaggtttttgtacacccagacagtggagtactaccactgtgactactttgactactggggccagggaaccctggtcaccgtctcctcaggtgagtcctcacaacctctctcctgctttaactctgaagggttttgctgcatttctggggggaaataagggtgctgggtctcctgccaagagagcccctgcagagggccaccctaggcctctggggtccaatgcccaacaacccccgggccctccccgggctcagtctgagagggtcccagggacgtagcggggcgccggtttttgtacacccagacagtggagtactaccactgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaggaggccagcagagggttccatgagaagggcaggacagggccacggacagtcagcttccatgtgacgcccggagacagaaggtctctgggtggctggtttttgtacacccagacagtggagtactaccactgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggacgctagcc

SEQ ID NO: 16 - DNA, 3365 bp, Artificial Sequence
  V67 vector sequence
ggatccactagccagtgtggtgcttaagtgcagatatcgcggccgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggggtcaaccgccatcctcgccctcctcctggctgttctccaaggagtctgtgccgaggtgcagctggtgcagtctggagcagaggtgaaaaagcccggggagtctctgaaaatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctccagtggagcagcctgaaggcctcggacaccgccatgtattactgtgcgagacacacagtggtagtactccactgtctgggtgtacaaaaacctccacaccgcaggtgcagaaactagccggaccagtgggcaccctcttccaggacagtcctcagtgatatcacatcgggaacccacatctggatcaggacggcacccagaacacaagatggcccatggggacagccccacagcccagcccttcccagacccctaaaaggtgtcccaccccctgcacctaccccaggactaaaaatccaggaggcctgactcctgcacatgctctgaccggatgtcacctcggcccctcctggaggggacaggagccctggagggtgagtcagaccctcctgccctcgacggcaggcggggaagattcagaccggtctgagatccccaggatgcagcaccactgtcaatgggggccccagacgcctggaccagcacctgcgtgggaaatgcctctgggctcactgaggggctttttgtgaaggccctcctgctatgtgactatggtgctaactaccacagtgatgaacccagcagcaaaaactgaccggactcccagggtttatgcacacttctcggctcagagttctccaggataagaagagccaggcccaaggatttctgcccagaccctcggcctctagggacaccttggccatgaaagcccatgggctggtgccccacacttcatctgccttcaaacaagggcttcagagggctctgaggtgacctcactcatgaccacaggtgcctgccagctgcaccgaaccctgtcccaacagctgccacagttccaacagccaattcctagggccgggaattgctgtagacaccagccttgttccagcacctcctgccaattgcctggattcccatcctggctggaatcaagagggcagcatccgcaagcttatgctcccccgggaccccgggctgtggtttttgtacacccagacagtggagtactaccactgtggctgaatacttccagcactggggccagggcaccctggtcaccgtctcctcaggtgagtctgctgtctggggatagcggggagccaggtgtactgggccaggcaagggctttgggctccttctccggctgtttgggaccacgttcagcagaaggcctttctttgggaactgggactctgctgctggggggcttcagacttggggacaggtgctcagcaaaggaggtcggcaggagggcggagggtggtttttgtacacccagacagtggagtactaccactgtgctactggtacttcgatctctggggccgtggcaccctggtcactgtctcctcaggtgagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgtgatgcttttgatatctggggccaagggacaatggtcaccgtctcttcaggtaagatggctttccttctgcctcctttctctgggcccagcgtcctctgtcctggagctgggagataatgtccgggggctccttggtctgcgctgggcaaagggtgggcagagtcatgcttgtgctggggacaaaatgaccttgggacacggggctggctgccacggccggcccgggacagtcggagagtcaggtttttgtacacccagacagtggagtactaccactgtgactactttgactactggggccagggaaccctggtcaccgtctcctcaggtgagtcctcacaacctctctcctgctttaactctgaagggttttgctgcatttctggggggaaataagggtgctgggtctcctgccaagagagcccctgcagagggccaccctaggcctctggggtccaatgcccaacaacccccgggccctccccgggctcagtctgagagggtcccagggacgtagcggggcgccggtttttgtacacccagacagtggagtactaccactgtgacaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaggaggccagcagagggttccatgagaagggcaggacagggccacggacagtcagcttccatgtgacgcccggagacagaaggtctctgggtggctggtttttgtacacccagacagtggagtactaccactgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggacgctagc

SEQ ID NO: 17 - DNA, 2158 bp, Artificial Sequence
  V86 antibody generating substrate sequence
ggatccactagccagtgtggtgcttaagtgcagatatcgcggccgcctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggagccgccaccatggggtcaaccgccatcctcgccctcctcctggctgttctccaaggagtctgtgccgaggtgcagctggtgcagtctggagcagaggtgaaaaagcccggggagtctctgaaaatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctccagtggagcagcctgaaggcctcggacaccgccatgtattactgtgcgagacacacagtggtagtactccactgtctgggtgtacaaaaacctccacaccgcaggtgcagaaactagccggaccagtgggcaccctcttccaggacagtcctcagtgatatcacatcgggaacccacatctggatcaggacggcacccagaacacaagatggcccatggggacagccccacagcccagcccttcccagacccctaaaaggtgtcccaccccctgcacctaccccaggactaaaaatccaggaggcctgactcctgcacatgctctgaccggatgtcacctcggcccctcctggaggggacaggagccctggagggtgagtcagaccctcctgccctcgacggcaggcggggaagattcagaccggtctgagatccccaggatgcagcaccactgtcaatgggggccccagacgcctggaccagcacctgcgtgggaaatgcctctgggctcactgaggggctttttgtgaaggccctcctgctatgtgactatggtgctaactaccacagtgatgaacccagcagcaaaaactgaccggactcccagggtttatgcacacttctcggctcagagttctccaggataagaagagccaggcccaaggatttctgcccagaccctcggcctctagggacaccttggccatgaaagcccatgggctggtgccccacacttcatctgccttcaaacaagggcttcagagggctctgaggtgacctcactcatgaccacaggtgcctgccagctgcaccgaaccctgtcccaacagctgccacagttccaacagccaattcctagggccgggaattgctgtagacaccagccttgttccagcacctcctgccaattgcctggattcccatcctggctggaatcaagagggcagcatccgcaagcttctgcctcctttctctgggcccagcgtcctctgtcctggagctgggagataatgtccgggggctccttggtctgcgctgggcaaagggtgggcagagtcatgcttgtgctggggacaaaatgaccttgggacacggggctggctgccacggccggcccgggacagtcggagagtcaggtttttgtacacccagacagtggagtactaccactgtgactactttgactactggggccagggaaccctggtcaccgtctcctcaggtgagtcctcacaacctctctcctgctttaactctgaagggttttgctgcatttctggggggaaataagggtgctgggtctcctgccaagagagcccctgcagagggccaccctaggcctctggggtccaatgcccaacaacccccgggccctccccgggctcagtctgagagggtcccgctagc

SEQ ID NO: 18 - DNA, 28 bp, Artificial Sequence
  Recombination signal sequence
cacagtgctccagggctgaacaaaaacc

SEQ ID NO: 19 - DNA, 39 bp, Artificial Sequence
  Recombination signal sequence
cacagtggtagtactccactgtctgggtgtacaaaaacc

SEQ ID NO: 20 - DNA, 28 bp, Artificial Sequence
  Recombination signal sequence
cacagtggtacagaccaatacaaaaacc

SEQ ID NO: 21 - DNA, 29 bp, Artificial Sequence
  Recombination signal sequence
cacatagcaggagggccttcacaaaaagc

SEQ ID NO: 22 - DNA, 28 bp, Artificial Sequence
  Recombination signal sequence
cacagtgatgaacccagcagcaaaaact

SEQ ID NO: 23 - DNA, 28 bp, Artificial Sequence
  Recombination signal sequence
cacagtaggaggggccttcacaaaaagc

SEQ ID NO: 24 - DNA, 28 bp, Artificial Sequence
  Recombination signal sequence
cacagtgatgaaactagcagcaaaaact

SEQ ID NO: 25 - DNA, 29 bp, Artificial Sequence
  Recombination signal sequence
cacatagcaggagggccttcacaaaaagc

SEQ ID NO: 26 - DNA, 28 bp, Artificial Sequence
  Recombination signal sequence
cacagtgatgaacccagcagcaaaaact

SEQ ID NO: 27 - DNA, 29 bp, Artificial Sequence
  Recombination signal sequence
cacatagcaggagggccttcacaaaaagc

SEQ ID NO: 28 - DNA, 28 bp, Artificial Sequence
  Recombination signal sequence
cacagtgatgaacccagcagcaaaaact

SEQ ID NO: 29 - DNA, 28 bp, Artificial Sequence
  Recombination signal sequence
cacagtgatacagaccttaacaaaaacc

SEQ ID NO: 30 - DNA, 39 bp, Artificial Sequence
  Recombination signal sequence
cacagtggtagtactccactgtctggctgtacaaaaacc

SEQ ID NO: 31 - DNA, 28 bp, Artificial Sequence
  Recombination signal sequence
cacagtgctacagactggaacaaaaacc

SEQ ID NO: 32 - DNA, 39 bp, Artificial Sequence; identical to SEQ ID NO: 30
  Recombination signal sequence

SEQ ID NO: 33 - DNA, 28 bp, Artificial Sequence
  Recombination signal sequence
cacagtgctccagggctgaacaaaaacc

SEQ ID NO: 34 - DNA, 39 bp, Artificial Sequence; identical to SEQ ID NO: 19
  Recombination signal sequence

SEQ ID NO: 35 - DNA, 28 bp, Artificial Sequence
  Recombination signal sequence
cacagtgctacagactggaacaaaaacc

SEQ ID NO: 36 - DNA, 39 bp, Artificial Sequence
  Recombination signal sequence
cacagtgttgcaaccacatcctgagtgtgtacaaaaacc

SEQ ID NO: 37 - DNA, 28 bp, Artificial Sequence
  Recombination signal sequence
cacagtgctacagactggaacaaaaacc

SEQ ID NO: 38 - DNA, 39 bp, Artificial Sequence; identical to SEQ ID NO: 30
  Recombination signal sequence

SEQ ID NO: 39 - DNA, 28 bp, Artificial Sequence
  Recombination signal sequence
cacagtgctacagactggaacaaaaacc

SEQ ID NO: 40 - DNA, 39 bp, Artificial Sequence
  Recombination signal sequence
cacagtgacggagataaaggaggaagcaggacaaaaacc

SEQ ID NO: 41 - DNA, 28 bp, Artificial Sequence
  Recombination signal sequence
cacagtggtacagaccaatacagaaacc

SEQ ID NO: 42 - DNA, 39 bp, Artificial Sequence
  Recombination signal sequence
cacagtggccgggccccgcggcccggcggcacaaaaacc

SEQ ID NO: 43 - DNA, 28 bp, Artificial Sequence
  Recombination signal sequence
cacggtgctacagactggaacaaaaacc

SEQ ID NO: 44 - DNA, 39 bp, Artificial Sequence; identical to SEQ ID NO: 30
  Recombination signal sequence

SEQ ID NO: 45 - DNA, 28 bp, Artificial Sequence
  Recombination signal sequence
cacaatgctacagactggaacaaaaacc

SEQ ID NO: 46 - DNA, 39 bp, Artificial Sequence; identical to SEQ ID NO: 30
  Recombination signal sequence

SEQ ID NO: 47 - DNA, 28 bp, Artificial Sequence
  Recombination signal sequence
cacagcgctacagactggaacaaaaacc

SEQ ID NO: 48 - DNA, 39 bp, Artificial Sequence; identical to SEQ ID NO: 30
  Recombination signal sequence

SEQ ID NO: 49 - DNA, 28 bp, Artificial Sequence
  Recombination signal sequence
cacagtgctacagactggaacaaaaacc

SEQ ID NO: 50 - DNA, 39 bp, Artificial Sequence
  Recombination signal sequence
cacaatggtagtactccactgtctggctgtacaaaaacc

SEQ ID NO: 51 - DNA, 28 bp, Artificial Sequence
  Recombination signal sequence
cacagtgctacagactggaacaaaaacc

SEQ ID NO: 52 - DNA, 39 bp, Artificial Sequence
  Recombination signal sequence
cacagcggtagtactccactgtctggctgtacaaaaacc

SEQ ID NO: 53 - DNA, 28 bp, Artificial Sequence
  Recombination signal sequence
cacagtgctacagactggaacaaaaacc

SEQ ID NO: 54 - DNA, 39 bp, Artificial Sequence
  Recombination signal sequence
cacagtagtagtactccactgtctggctgtacaaaaacc

SEQ ID NO: 55 - DNA, 28 bp, Artificial Sequence
  Recombination signal sequence
cacagtgctacagactggaacaaaaacc

SEQ ID NO: 56 - DNA, 39 bp, Artificial Sequence
  Recombination signal sequence
cacagtggtagtactccactgtctggctgtacaataacc

SEQ ID NO: 57 - DNA, 28 bp, Artificial Sequence
  Recombination signal sequence
cacagtgctacagactggaacaaaaacc

SEQ ID NO: 58 - DNA, 39 bp, Artificial Sequence
  Recombination signal sequence
cacagtggtagtactccactgtctggctgtacaagaacc

SEQ ID NO: 59 - DNA, 28 bp, Artificial Sequence
  Recombination signal sequence
cacagtgctacagactggaacaaaaacc

SEQ ID NO: 60 - DNA, 39 bp, Artificial Sequence
  Recombination signal sequence
cacagtggtagtactccactgtctggctgtacacgaacc

SEQ ID NO: 61 - DNA, 28 bp, Artificial Sequence
  Recombination signal sequence
cacagtgctacagactggacaaaaaccc

SEQ ID NO: 62 - DNA, 39 bp, Artificial Sequence; identical to SEQ ID NO: 30
  Recombination signal sequence

SEQ ID NO: 63 - DNA, 28 bp, Artificial Sequence
  Recombination signal sequence
cacagtgctacagactggaacaaaaacc

SEQ ID NO: 64 - DNA, 39 bp, Artificial Sequence; identical to SEQ ID NO: 60
  Recombination signal sequence

SEQ ID NO: 65 - DNA, 28 bp, Artificial Sequence
  Recombination signal sequence
cacaatgctacagactggaacaaaaacc

SEQ ID NO: 66 - DNA, 39 bp, Artificial Sequence; identical to SEQ ID NO: 50
  Recombination signal sequence

SEQ ID NO: 67 - DNA, 28 bp, Artificial Sequence
  Recombination signal sequence
cacagcgctacagactggaacaaaaacc

SEQ ID NO: 68 - DNA, 39 bp, Artificial Sequence; identical to SEQ ID NO: 52
  Recombination signal sequence

SEQ ID NO: 69 - DNA, 28 bp, Artificial Sequence
  Recombination signal sequence
tacagtgctacagactggaacaaaaacc

SEQ ID NO: 70 - DNA, 39 bp, Artificial Sequence; identical to SEQ ID NO: 54
  Recombination signal sequence

SEQ ID NO: 71 - DNA, 28 bp, Artificial Sequence
  Recombination signal sequence
gacagtgctacagactggaacaaaaacc

SEQ ID NO: 72 - DNA, 39 bp, Artificial Sequence; identical to SEQ ID NO: 30
  Recombination signal sequence

SEQ ID NO: 73 - DNA, 28 bp, Artificial Sequence
  Recombination signal sequence
catagtgctacagactggaacaaaaacc

SEQ ID NO: 74 - DNA, 39 bp, Artificial Sequence; identical to SEQ ID NO: 50
  Recombination signal sequence

SEQ ID NO: 75 - DNA, 28 bp, Artificial Sequence
  Recombination signal sequence
cacaatgctacagactggaacaaaaacc

SEQ ID NO: 76 - DNA, 39 bp, Artificial Sequence
  Recombination signal sequence
catagtggtagtactccactgtctggctgtacaaaaacc

SEQ ID NO: 77 - DNA, 28 bp, Artificial Sequence
  Recombination signal sequence
cacagtgctacagactggaacaaaaacc

SEQ ID NO: 78 - DNA, 39 bp, Artificial Sequence
  Recombination signal sequence
cacagtggtagtactccactgtctggctgttgtctctga

SEQ ID NO: 79 - DNA, 28 bp, Artificial Sequence
  Recombination signal sequence
cagagtgctccagggctgaacaaaaacc

SEQ ID NO: 80 - DNA, 39 bp, Artificial Sequence; identical to SEQ ID NO: 19
  Recombination signal sequence

SEQ ID NO: 81 - DNA, 28 bp, Artificial Sequence
  Recombination signal sequence
cacagtgctccagggctgaaaaaaaacc

SEQ ID NO: 82 - DNA, 39 bp, Artificial Sequence; identical to SEQ ID NO: 19
  Recombination signal sequence

SEQ ID NO: 83 - DNA, 28 bp, Artificial Sequence
  Recombination signal sequence
ctcagtgctccagggctgaacaaaaacc

SEQ ID NO: 84 - DNA, 39 bp, Artificial Sequence; identical to SEQ ID NO: 19
  Recombination signal sequence

SEQ ID NO: 85 - DNA, 17 bp, Artificial Sequence
  D segment encoding sequence
ggtacaactggaacgac

SEQ ID NO: 86 - DNA, 17 bp, Artificial Sequence
  D segment encoding sequence
ggtataactggaactac

SEQ ID NO: 87 - DNA, 17 bp, Artificial Sequence
  D segment encoding sequence
ggtataactggaacgac

SEQ ID NO: 88 - DNA, 20 bp, Artificial Sequence
  D segment encoding sequence
ggtatagtgggagctactac

SEQ ID NO: 89 - DNA, 31 bp, Artificial Sequence
  D segment encoding sequence
aggatattgtagtagtaccagctgctatacc

SEQ ID NO: 90 - DNA, 31 bp, Artificial Sequence
  D segment encoding sequence
aggatattgtactaatggtgtatgctatacc

SEQ ID NO: 91 - DNA, 31 bp, Artificial Sequence
  D segment encoding sequence
aggatattgtagtggtggtagctgctactcc

SEQ ID NO: 92 - DNA, 28 bp, Artificial Sequence
  D segment encoding sequence
agcatattgtggtggtgactgctattcc

SEQ ID NO: 93 - DNA, 31 bp, Artificial Sequence
  D segment encoding sequence
gtattacgatttttggagtggttattatacc

SEQ ID NO: 94 - DNA, 31 bp, Artificial Sequence
  D segment encoding sequence
gtattacgatattttgactggttattataac

SEQ ID NO: 95 - DNA, 31 bp, Artificial Sequence
  D segment encoding sequence
gtattactatggttcggggagttattataac

SEQ ID NO: 96 - DNA, 37 bp, Artificial Sequence
  D segment encoding sequence
gtattatgattacgtttgggggagttatcgttatacc

SEQ ID NO: 97 - DNA, 31 bp, Artificial Sequence
  D segment encoding sequence
gtattactatgatagtagtggttattactac

SEQ ID NO: 98 - DNA, 16 bp, Artificial Sequence
  D segment encoding sequence
tgactacagtaactac

SEQ ID NO: 99 - DNA, 16 bp, Artificial Sequence
  D segment encoding sequence
tgactacagtaactac

SEQ ID NO: 100 - DNA, 16 bp, Artificial Sequence
  D segment encoding sequence
tgactacggtgactac

SEQ ID NO: 101 - DNA, 19 bp, Artificial Sequence
  D segment encoding sequence
tgactacggtggtaactcc

SEQ ID NO: 102 - DNA, 20 bp, Artificial Sequence
  D segment encoding sequence
gtggatacagctatggttac

SEQ ID NO: 103 - DNA, 23 bp, Artificial Sequence
  D segment encoding sequence
gtggatatagtggctacgattac

SEQ ID NO: 104 - DNA, 20 bp, Artificial Sequence
  D segment encoding sequence
gtggatacagctatggttac

SEQ ID NO: 105 - DNA, 20 bp, Artificial Sequence
  D segment encoding sequence
gtagagatggctacaattac

SEQ ID NO: 106 - DNA, 18 bp, Artificial Sequence
  D segment encoding sequence
gagtatagcagctcgtcc

SEQ ID NO: 107 - DNA, 21 bp, Artificial Sequence
  D segment encoding sequence
gggtatagcagcagctggtac

SEQ ID NO: 108 - DNA, 21 bp, Artificial Sequence
  D segment encoding sequence
gggtatagcagtggctggtac

SEQ ID NO: 109 - DNA, 11 bp, Artificial Sequence
  D segment encoding sequence
ctaactgggga

SEQ ID NO: 110 - protein, 41 aa, Artificial sequence
  Single domain A avimer construct
CAPSQFQCGSGYCISQRWVCDGENDCEDGSDEANCAGSVPT

SEQ ID NO: 111 - DNA, 27 bp, Artificial sequence
  Cassette for generating avimer sequence diversity
agccagttccagtgcggctccggctac

SEQ ID NO: 112 - protein, 9 aa, Artificial sequence
  Cassette for generating avimer sequence diversity
SQFQCGSGY

SEQ ID NO: 113 - DNA, 33 bp, Artificial sequence
  Cassette for generating avimer sequence diversity
tacagccagtttgtgtgcggctccggctactac

SEQ ID NO: 114 - DNA, 2425 bp, Artificial sequence
  Avimer construct E188 (partial sequence)
gccgccaccatggagtttgggctgagctggctttttcttgtggctattttaaaaggtgtccagtgttacccatacgatgttccagattacgcttgtgcccctcacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagcgtctctccaagtgcaaagggacaggaggtttttgttaagggctgtatcactgtgagccagttccagtgcggctccggctaccacagtgatacagcccttaacaaaaacccctactgcaacctggcggtaagagacgtccggaggccagcccttctcatgttcagagaacatggttaactggttaagtcatgtcgtcccacaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgggctactgcatcagccagagatgggtgtgcgacggggagaatgattgcgaggacggcagcgacgaggccaattgtgccggctctgtgcctaccgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggcaaactggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatca

SEQ ID NO: 115 - DNA, 2533 bp, Artificial sequence
  Avimer construct E189 (partial sequence)
gccgccaccatggagtttgggctgagctggctttttcttgtggctattttaaaaggtgtccagtgttacccatacgatgttccagattacgcttgcctgccccacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagcgtctctccaagtgcaaagggacaggaggtttttgttaagggctgtatcactgtggaccagttcagatgcggcaacggccagtgcatccccctggattgggtgtgcgacggcgtgaacgactgccccgattccgatgaggaaggctgcccccctagaacctgtgcccctagccagcacagtgatacagcccttaacaaaaacccctactgcaacctggcggtaagagacgtccggaggccagcccttctcatgttcagagaacatggttaactggttaagtcatgtcgtcccacaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgttccagtgcggctccggctactgcatcagccagagatgggtgtgcgacggggagaatgattgcgaggacggcagcgacgaggccaattgtgccggctctgtgcctaccgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggcaaactggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatca

SEQ ID NO: 116 - DNA, 7611 bp, Artificial sequence
  Avimer construct E188 (complete sequence)
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggagtttgggctgagctggctttttcttgtggctattttaaaaggtgtccagtgttacccatacgatgttccagattacgcttgtgcccctcacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagcgtctctccaagtgcaaagggacaggaggtttttgttaagggctgtatcactgtgagccagttccagtgcggctccggctaccacagtgatacagcccttaacaaaaacccctactgcaacctggcggtaagagacgtccggaggccagcccttctcatgttcagagaacatggttaactggttaagtcatgtcgtcccacaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgggctactgcatcagccagagatgggtgtgcgacggggagaatgattgcgaggacggcagcgacgaggccaattgtgccggctctgtgcctaccgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggcaaactggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttaaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 117 - DNA, 7719 bp, Artificial sequence
  Avimer construct E188 (complete sequence)
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggagtttgggctgagctggctttttcttgtggctattttaaaaggtgtccagtgttacccatacgatgttccagattacgcttgcctgccccacagtggtagtactccactgtctgggtgtacaaaaacctccctgcacgcctctctaacctcacaattctgtggcggccgcgccgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggtgagtacaggaggtggagagtacgcgtaacacttaagcgtctctccaagtgcaaagggacaggaggtttttgttaagggctgtatcactgtggaccagttcagatgcggcaacggccagtgcatccccctggattgggtgtgcgacggcgtgaacgactgccccgattccgatgaggaaggctgcccccctagaacctgtgcccctagccagcacagtgatacagcccttaacaaaaacccctactgcaacctggcggtaagagacgtccggaggccagcccttctcatgttcagagaacatggttaactggttaagtcatgtcgtcccacaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgactgcaggagtcccactgcacccccctcccagtcttctctgtccaggcaccaggccaggtatctggggtgtgcagccggcctgggtctggcctgaggccacaagcccgggggtctgtgtggctggggacagggacgccggctgcctctgctctgtgcttgggccatgtgacccattcgagtgtcctgcacgggcacaggtttttgtacacccagacagtggagtactaccactgtgttccagtgcggctccggctactgcatcagccagagatgggtgtgcgacggggagaatgattgcgaggacggcagcgacgaggccaattgtgccggctctgtgcctaccgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggcaaactggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttaaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 118 - DNA, 6139 bp, Artificial sequence
  Acceptor vector
ctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacgacgttgtaaaacgacggccagtgagcgcgacgtaatacgactcactatagggcgaattggcggaaggccgtcaaggcctaggcgcgcctgaataacttcgtatagcatacattatagcaatttatcgaaaaagcctgaactcaccgcgacatccgtggagaaattcctcatcgaaaaattcgactccgtgtccgatctcatgcagctgtccgagggcgaggagagtagagcattctcattcgatgtgggcgggagaggctacgtgctgagagtgaactcttgtgccgacggcttctacaaggaccgatacgtctaccggcattttgcttccgccgctctgcctattccagaagtcctggacattggggagtttagcgagtccctcacttactgtattagccggcgagcccagggagtgacactccaggatctgcctgaaactgaactgcctgctgtgctccagcctgtcgctgaggcaatggatgctattgctgctgccgatctgagtcagactagcggattcggcccatttggaccccagggcattggccagtacacaacatggcgagacttcatctgtgctatcgccgatcctcacgtgtaccattggcagactgtgatggacgatactgtgtctgcttctgtggcacaggcactcgacgaactcatgctgtgggctgaggactgtcctgaagtgagacatctggtccatgccgattttggctccaacaatgtgctcaccgataacgggagaatcactgccgtgatcgactggagcgaggcaatgtttggcgattcccagtacgaagtggccaacatcttcttttggcggccttggctggcttgtatggaacagcagacccggtactttgaacggcgccaccctgagctggctgggagtcctagactgagagcctacatgctccgaattggcctggatcagctctaccagtcactggtggatggcaatttcgacgatgctgcttgggcacaggggcgctgtgatgctattgtccgatccggcgctggaactgtggggagaacacagatcgctaggagatccgctgctgtctggaccgatggatgtgtggaagtgctggccgatagtggaaaccggaggccttcaacccgaccccgggcaaaggagtaatgaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcccgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactgctcgacgatctgatcaagagacaggataaggagccgccaccatggagtttgggctgagctggctttttcttgtggctattttaaaaggtgtccagtgtagagaccggaagagattggtaccgagcccaaatcttgtgacaaaactcacacatgcccaccgtgcccagcacctgaactcctggggggaccgtcagtcttcctcttccccccaaaacccaaggacaccctcatgatctctagaacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggcaaactggctctcattgtcctgggcggcgtggctggcctgctgctgtttattgggctgggcatcttcttttgtgtccggtgtcggcataggaggcgccaaggaggtggcggatctggagggggaggatctggagggggctcaggatcagggggaggatctggaggcggatcaactgagtacaaacccactgtgaggctcgctactagagatgatgtgcctagagctgtccgaactctggctgctgccttcgccgattaccctgccactcgccataccgtcgatcccgatcgccacattgaacgagtcaccgaactccaggagctgtttctcactagagtcgggctggatattggcaaagtctgggtggccgatgacggagccgctgtcgctgtgtggactacacctgagtctgtggaggctggcgccgtgtttgctgaaattggacctcggatggctgaactgtctggatctcgactggctgcccagcagcagatggagggactgctggcaccccatagaccaaaggaacctgcctggtttctggcaactgtgggagtgtcacccgatcatcagggcaaaggactgggatctgccgtggtgctccctggcgtggaggccgctgaacgagctggcgtccccgcttttctcgaaacttctgccccccgaaatctccctttctacgaacgactgggattcactgtcaccgccgatgtcgaagtgcctgaggggcctagaacatggtgtatgacccggaaacccggagcttaaccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctcgagttaattaactggcctcatgggccttccgctcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 119 - DNA, 29 bp, Artificial sequence
  Primer
tcttggcattatgcacctccacgccgtcc

SEQ ID NO: 120 - DNA, 49 bp, Artificial sequence
  Primer
gagagagattggtctcgagaacccactgcttactgctcgacgatctgat

SEQ ID NO: 121 - DNA, 30 bp, Artificial sequence
  Primer
gtcttcgtggctcacgtccaccaccacgca

SEQ ID NO: 122 - DNA, 29 bp, Artificial sequence
  Primer
ctgacctggttcttggtcagctcatcccg

SEQ ID NO: 123 - DNA, 10 bp, Artificial sequence
  Avimer variant sequence
agggccaaga

SEQ ID NO: 124 - DNA, 14 bp, Artificial sequence
  Avimer variant sequence
tggggttaagcctc

SEQ ID NO: 125 - DNA, 14 bp, Artificial sequence
  Avimer variant sequence
tagggggttccagt

SEQ ID NO: 126 - DNA, 16 bp, Artificial sequence
  Avimer variant sequence
ccctccgtcctacctc

SEQ ID NO: 127 - DNA, 18 bp, Artificial sequence
  Avimer variant sequence
tccagtgcggctccggga

SEQ ID NO: 128 - DNA, 17 bp, Artificial sequence
  Avimer variant sequence
ggagccgcactggaact

SEQ ID NO: 129 - protein, 6 aa, Artificial sequence
  Avimer variant sequence
DYACAP

SEQ ID NO: 130 - protein, 9 aa, Artificial sequence
  Avimer variant sequence
SQFQCGSGY

SEQ ID NO: 131 - protein, 11 aa, Artificial sequence
  Avimer variant sequence
GYCISQRWVCD

SEQ ID NO: 132 - protein, 10 aa, Artificial sequence
  Avimer variant sequence
FQFQCGSGYN

SEQ ID NO: 133 - protein, 9 aa, Artificial sequence
  Avimer variant sequence
CISQRWVCD

SEQ ID NO: 134 - protein, 9 aa, Artificial sequence
  Avimer variant sequence
TSSSAAPAY

SEQ ID NO: 135 - protein, 10 aa, Artificial sequence
  Avimer variant sequence
RRQFQCGSGY

SEQ ID NO: 136 - protein, 10 aa, Artificial sequence
  Avimer variant sequence
YCISQRWVCD

SEQ ID NO: 137 - protein, 11 aa, Artificial sequence
  Avimer variant sequence
LLASSSAAPAT

SEQ ID NO: 138 - protein, 8 aa, Artificial sequence
  Avimer variant sequence
QDAAPATS

SEQ ID NO: 139 - protein, 9 aa, Artificial sequence
  Avimer variant sequence
PQFQCGSGY

SEQ ID NO: 140 - protein, 5 aa, Artificial sequence
  Avimer variant sequence
SSSSD

SEQ ID NO: 141 - protein, 8 aa, Artificial sequence
  Avimer variant sequence
RSRSRTGT

SEQ ID NO: 142 - protein, 8 aa, Artificial sequence
  Avimer variant sequence
ASSSAAPA

SEQ ID NO: 143 - protein, 8 aa, Artificial sequence
  Avimer variant sequence
RFQCGSGS

SEQ ID NO: 144 - protein, 11 aa, Artificial sequence
  Avimer variant sequence
RRQFQCGSGFP

SEQ ID NO: 145 - protein, 9 aa, Artificial sequence
  Avimer variant sequence
QFQCGSGYD

SEQ ID NO: 146 - protein, 9 aa, Artificial sequence
  Avimer variant sequence
RAKRLWGAS

SEQ ID NO: 147 - protein, 9 aa, Artificial sequence
  Avimer variant sequence
SQFQCGSGY

SEQ ID NO: 148 - protein, 10 aa, Artificial sequence
  Avimer variant sequence
RQFQCGSGYG

SEQ ID NO: 149 - protein, 10 aa, Artificial sequence
  Avimer variant sequence
LGGSSAAPAE

SEQ ID NO: 150 - protein, 11 aa, Artificial sequence
  Avimer variant sequence
RTVPVPLRPTS

SEQ ID NO: 151 - protein, 9 aa, Artificial sequence
  Avimer variant sequence
SGDSQFQCH

SEQ ID NO: 152 - protein, 9 aa, Artificial sequence
  Avimer variant sequence
PSSSSAAPG

SEQ ID NO: 153 - protein, 9 aa, Artificial sequence
  Avimer variant sequence
LQFQCGSGF

SEQ ID NO: 154 - protein, 9 aa, Artificial sequence
  Avimer variant sequence
LASSSAAPA

SEQ ID NO: 155 - protein, 10 aa, Artificial sequence
  Cassette for generating avimer sequence diversity
QPVCVRLRLL

SEQ ID NO: 156 - protein, 10 aa, Artificial sequence
  Cassette for generating avimer sequence diversity
TASLCAAPAT

SEQ ID NO: 157 - protein, 11 aa, Artificial sequence
  Cassette for generating avimer sequence diversity
YSQFVCGSGYY

(19) United States
(12) Patent Application Publication
(10) Pub. No.: US 2013/0236931 A1
(43) Pub. Date: Sep. 12, 2013

(54) SEQUENCE DIVERSITY GENERATION IN IMMUNOGLOBULINS AND OTHER PROTEINS

(71) Applicants: Michael Gallo, North Vancouver (CA); Jaspal Singh Kang, Surrey (CA); Craig Robin Pigott, Vancouver (CA)

(72) Inventors: Michael Gallo, North Vancouver (CA); Jaspal Singh Kang, Surrey (CA); Craig Robin Pigott, Vancouver (CA)

(21) Appl. No.: 13/765,484

(22) Filed: Feb. 12, 2013

Related U.S. Application Data
(63) Continuation-in-part of application No. 13/205,218, filed on Aug. 8, 2011.
Application No. 13/205,218 is a continuation of application No. 12/423,594, filed on Apr. 14, 2009, now Pat. No. 8,012,714.
(60) Provisional application No. 61/044,795, filed on Apr. 14, 2008.

(51) Int. Cl. C12P 21/00

(52) U.S. Cl. CPC C12P 21/00
USPC 435/69.6; 435/325; 435/358; 435/369; 435/366; 435/363; 435/352; 435/354; 435/353; 435/350; 435/351; 435/326; 435/69.1

(57) ABSTRACT

An in vitro system for generating sequence, and thus structural, diversity in proteins is described. The system can be constructed using appropriately selected nucleic acid molecules that encode regions of a selected protein or proteins and recombination signal sequences (RSS). The selected protein(s) can be, for example, immunoglobulin (Ig) V, D, J and/or C regions, regions of a non-immunoglobulin (non-Ig) protein, or a combination of Ig regions and non-Ig regions. Assembly of such appropriately selected components and their introduction into suitable recombination-competent host cells allows for recombination between the RSS sequences and introduction of sequence and structural diversity into the protein(s).

### FIELD OF THE INVENTION

The present invention relates generally to compositions and methods for use in generating protein sequence diversity and in particular, to an in vitro molecular biological approach to generating proteins having structurally diverse regions and other advantageous properties.

### BACKGROUND OF THE INVENTION

The recombination of different immunoglobulin heavy chain (IgH) V, D, and J gene segments creates a wide repertoire of antibody variable regions having distinct binding specificities for different antigens. Antibody light chains (Kappa and Lambda) are also generated via the same type of recombination process except that the light chain does not have any D gene segments. These recombination events involve the breaking and joining of DNA segments in the genome and collectively referred to as V(D)J recombination.

V(D)J recombination occurs at two steps. First, two lymphoid-specific recombinase proteins that are expressed in cells which are capable of immunoglobulin gene rearrangement (e.g., pre-B lymphocytes), RAG-1 and RAG-2, recognize signal sequences and form a synaptic complex with the assistance of HMG1, one of the non-histone chromatin proteins. Then, the RAG proteins cut DNA at the border between the signal sequence and the immunoglobulin polypeptide-coding sequence. At this cleavage step, DNA is nicked first by RAG proteins at the top strand, and then the 3′-hydroxyl group attacks the phosphodiester bond of the bottom strand by a direct nucleophilic reaction, resulting in formation of a hairpin intermediate at the coding end.

The recombination signal sequence (RSS) consists of two conserved sequences (heptamer, 5′-CACAGTG-3′, and nonamer, 5′-ACAAAAACC-3′), separated by a spacer of either 12+/−1 bp (“12-signal”) or 23+/−1 bp (“23-signal”). To begin this lymphoid-specific process, two signals (one 12-signal and one 23-signal) are selected and rearranged under the “12/23 rule”; recombination does not occur between two RSS signals with the same size spacer. In spite of the specificity of the recombinase most of the nucleotide positions within the recombination signals are variable, especially those in the 23 signal. The consensus sequences being accepted as CACAGTG for the heptamer and ACAAAAACC for the nonamer. A number of nucleotide positions have been identified as important for recombination including the CA dinucleotide at position one and two of the heptamer, and a C at heptamer position three has also been shown to be strongly preferred as well as an A nucleotide at positions 5, 6, 7 of the nonamer. (Ramsden et. al 1994; Akamatsu et. al. 1994; Hesse et. al. 1989). Mutations of other nucleotides have minimal or inconsistent effects. The spacer, although more variable, also has an impact on recombination, and single-nucleotide replacements have been shown to significantly impact recombination efficiency (Fanning et. al. 1996, Larijani et. al 1999; Nadel et. al. 1998). Because of the large amount of sequence variability found at functional RSSs it is difficult to comprehensively evaluate the influence of specific sequences on recombination potential. Recently the Schatz laboratory developed genetic and functional screens to evaluate several thousand 12 spacer RSSs in the context of a consensus heptamer and non-consensus nonamer. They were able to demonstrate that non-consensus spacer nucleotides often impaired recombination (Lee et. al. 2003). It is believed that the spacer might influence recombination at a post-cleavage stage, perhaps during formation of the synaptic complex or coding joint resolution. Differences in the spacer can account for over a 30-fold range in recombination efficiency (Cowell et. al 2004). Studies have shown that the nonamer may be the primary determinant of RSS binding by the recombinase while the heptamer sequence guides cleavage.

The final recombination potential of any single RSS is the combination of all its sequences, which has made predictions difficult. Cowell et al. have generated an algorithm and have identified the optimal sequences for high efficiency recombination. Other in vitro studies have defined the minimal distance required between signal sequences as well as the influence of flanking coding sequences on recombination efficiency. Although it is difficult to predict the efficiency of a RSS by its sequence alone, an algorithm of good predictive potential has been generated and there are empirical data on specific RSSs on the basis of which a skilled person can select RSS polynucleotide sequences that would have significantly different recombination efficiencies (Ramsden et. al 1994; Akamatsu et. al. 1994; Hesse et. al. 1989 and Cowell et. al. 1994).

Following the (RSS) signal-directed DNA cleavage the broken DNA ends are repaired by double-strand break repair proteins. The coding ends are often processed before being repaired, which is an additional step that generates more potential for structural diversity from the reaction. Such processing involves deletion of nucleotides at the coding joint of antigen receptor genes, which is commonly observed at the V_{H} 3′ junction, at both sides (5′ and 3′) of the D segment, and at the 5′ junction of the J segment, followed in some cases by addition of other nucleotides at these processing sites. Terminal deoxynucleotide transferase (TdT) has been identified as a polymerase that plays a role in such nucleotide addition during V(D)J recombination, thus contributing further diversity to the antibody repertoire (Landau et al., Mol. Cell Biol. 1987 7:3237). The diversity of the antibody repertoire is therefore the combined result of (i) different gene segment utilization through the recombination events, (ii) optional deletion and/or addition of one or more nucleotides at each of the junctions (e.g., mediation of junctional diversity, such as by TdT), and (iii) differential pairings of the various heavy and light chain combinations that may result from (i) and (ii) in different cells. In vivo the process is highly regulated and once a set of gene segments for a specific antigen receptor is successfully rearranged to generate a functional molecule the gene rearrangement process for additional antigen receptors is prohibited within a given lymphocyte; once successful heavy chain rearrangement is achieved no additional rearrangements take place at that locus. (Inlay et. al. 2006; Alt et. al. 1984)

Protein function can be modified and improved in vitro by a variety of methods, including site-directed mutagenesis, combinatorial cloning and random mutagenesis combined with an appropriate selection system.

The method of random mutagenesis together with selection has been used in a number of cases to improve protein function and generally follows one of two strategies. The first involves randomisation of the entire gene sequence in combination with the selection of a variant (mutant) protein with desired characteristics. This process can be repeated on the selected variant until a protein variant is found which is considered optimal. Mutations are typically introduced by error-prone PCR (Leung et al., 1989, Technique, 1:11-15) with a mutation rate of approximately 0.7%. The second strategy is to mutagenize defined regions of the gene with degenerate primers (“saturation mutagenesis”), which allows for mutation rates of up to 100% (Griffiths et al., 1994, EMBO. J, 13:3245-3260; Yang et al., 1995, J. Mol. Biol. 254:392-403), followed by selection of variants with interesting characteristics. The mutated DNA regions from different variants, each with interesting characteristics, may subsequently be combined into one coding sequence (Yang et al., ibid).

Another process for in vitro mutation of protein function is “DNA shuffling,” which uses random fragmentation of DNA and assembly of fragments into a functional coding sequence (Stemmer, 1994, Nature 370:389-391). The DNA shuffling process generates diversity by recombination, combining useful mutations from individual genes. The genes are randomly fragmented using DNase I and then reassembled by recombination with each other. The starting material can be either a single gene (first randomly mutated using error-prone PCR) or naturally occurring homologous sequences (so-called family shuffling).

The use of “protein scaffolds” for the generation of novel binding proteins via combinatorial engineering has recently emerged as a powerful alternative to natural or recombinant antibodies. It has been found that novel binding sites can be introduced into proteins from several protein families with non-Ig architectures by combinatorial engineering, such as site-directed random mutagenesis combined with phage display or other selection techniques (Rothe, A., et al., 2006, FASEB J., 20:1599-1610). This concept requires a stable protein architecture (“scaffold”) tolerating multiple substitutions or insertions at the primary structural level (see reviews by Binz, H. K., et al., 2005, Nature Biotechnology, 23(10):1257-1268; Nygren, P-A. & Skerra, A., 2004, J. Immunol. Methods, 290:3-28, and Gebauer, M. & Skerra, A., 2009, Curr. Op. Chem. Biol., 13:245-255).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to sequence diversity generation in immunoglobulins and other proteins.

In accordance with one aspect of the invention, there is provided an isolated recombination-competent host cell comprising a nucleic acid composition for generating protein structural diversity comprising a tripartite recombination substrate, wherein the tripartite recombination substrate comprises: (a) a first nucleic acid sequence operably linked to an expression control sequence and consisting essentially of (i) a first polynucleotide sequence that encodes at least a first portion of a protein, and (ii) a first recombination signal sequence located 3′ to the first polynucleotide sequence; (b) a second nucleic acid sequence consisting essentially of (i) a second polynucleotide sequence that encodes at least a second portion of a protein, (ii) a second recombination signal sequence located 5′ to the second polynucleotide sequence that is capable of functional recombination with the first recombination signal sequence, and (iii) a third recombination signal sequence located 3′ to the second polynucleotide sequence; and (c) a third nucleic acid sequence consisting essentially of (i) a third polynucleotide sequence that encodes at least a third portion of a protein, and (ii) a fourth recombination signal sequence located 5′ to the third polynucleotide sequence that is capable of functional recombination with the third recombination signal sequence, wherein the tripartite recombination substrate can undergo recombination in the isolated host cell to form a recombined polynucleotide that encodes a structurally diversified protein, and wherein the isolated host cell expresses the structurally diversified protein, and wherein at least one of the first, second and third portions is a portion of a non-immunoglobulin protein.

In accordance with certain embodiments, the first, second and third portions are each a portion of a non-immunoglobulin protein.

In accordance with certain embodiments, the first, second and third portions are each a portion of the same non-immunoglobulin protein.

In accordance with certain embodiments, at least one of the first, second and third portions is a portion of an immunoglobulin protein.

In accordance with certain embodiments, the nucleic acid composition further comprises a fourth nucleic acid sequence that comprises a polynucleotide sequence encoding a membrane anchor domain operably linked to the tripartite recombination substrate, and wherein the expressed protein comprises a membrane anchor domain.

In accordance with certain embodiments, the nucleic acid composition is maintained extrachromosomally in the isolated host cell.

In accordance with certain embodiments, the nucleic acid composition is integrated into the genome of the isolated host cell.

In accordance with another aspect of the invention, there is provided a method for generating structural diversity in a protein comprising maintaining an isolated host cell as described above under conditions and for a time sufficient to allow for recombination of the tripartite recombination substrate and expression of the recombined polynucleotide, thereby generating a structurally diversified protein.

### BRIEF DESCRIPTION OF THE DRAWINGS

FIG. 1 shows theoretical Ig V_{H} locus D segment utilization by (FIG. 1A) locus having 50 functional V_{H}, 25 functional D and 6 functional J_{H} gene segments; and (FIG. 1B) theoretical Ig V_{H} locus having 21 functional V_{H}, 18 functional D and 6 functional J_{H} gene segments.

FIG. 2 shows theoretical Ig V_{H} locus D segment utilization by (FIG. 2A) locus having 6 functional V_{H}, 12 functional D and 6 functional J_{H} gene segments; (FIG. 2B) theoretical Ig V_{H} locus having 12 functional V_{H}, 12 functional D and 12 functional J_{H} gene segments; (FIG. 2C) theoretical Ig V_{H} locus having 13 functional V_{H}, 10 functional D and 9 functional J_{H} gene segments.

FIG. 3 shows a schematic diagram of the LacZ-RSS. The RSS with the 12 base pair recombination signal sequence and the RSS with the 23 base pair rescombination signal sequence are positioned in the same orientation. The HindIII-XhoI fragment of LacZ-RSS was inserted into pcDNA3.1(+) so that the LacZ open reading frame is in the opposite orientation relative to the CMV promoter to create vector V25. V25 is an inversional VDJ substrate.

FIG. 4 shows RAG-1/RAG-2 mediated recombination of a β-gal substrate (LacZ-RSS). 293 Cells were transfected with 67 ng of the LacZ-RSS plasmid, 0 (diamonds) or 33 ng (squares) of the RAG-2 plasmid and 0, 8, 17, 33 or 67 ng of the RAG-1 plasmid. Carrier plasmid was added such that all samples received the same total amount of DNA. Two days after transfection, cell lysates were prepared and beta-galactosidase activity was determined using the colorimetric substrate chlorophenol red-β-D-galactopyranoside (Sigma, St. Louis, Mo., Cat. No. 59767-25MG-F).

FIG. 5 shows a schematic diagram of ITS-4, a vector encoding a functional immunoglobulin kappa antibody light chain protein.

FIG. 6 shows a schematic diagram of ITS-6, a vector encoding a functional immunoglobulin IgG heavy chain membrane-expressed protein.

FIG. 7 shows a schematic diagram of V64, a tripartite immunoglobulin diversifying vector with a 2:1:6 (V:D:J) ratio.

FIG. 8 shows a schematic diagram of V67, a tripartite immunoglobulin diversifying vector with a 1:1:6 (V:D:J) ratio.

FIG. 9 shows a schematic diagram of V86, a tripartite immunoglobulin diversifying vector with a 1:1:1 (V:D:J) ratio.

FIG. 10 presents a schematic representation of (A) a single domain A avimer construct comprising a pair of RSSs in loop 1 and a pair of RSSs in loop 2, a selectable marker was included between the Tm domain and the poly A; (B) sequence details of the construct shown in (A) with arrows indicting the positions of insertion of the RSS cassettes, and (C) an overview of the steps for mutagenesis of the single domain A avimer construct shown in (A).

FIG. 11 presents a schematic representation of an overview of the steps for mutagenesis of a double domain A avimer construct including RSS sequences in each loop 1.

FIG. 12 presents a partial nucleotide sequence of avimer construct E188 that comprises a single avimer A domain, a pair of RSSs introduced into loop 1 of the construct and a pair of RSSs introduced into loop 2 of the construct together with flanking sequences encoding GY amino acid residues [SEQ ID NO:114].

FIG. 13 presents a partial nucleotide sequence of avimer construct E189 that comprises double avimer A domains and a pair of RSSs in each loop 1 of the construct, as well as stop codons in other reading frames in the 3′ loop 1.1 to 5′ loop 1.2 region [SEQ ID NO:115].

FIG. 14 presents the nucleotide sequence for the vector E188 [SEQ ID NO:116].

FIG. 15 presents the nucleotide sequence for the vector E189 [SEQ ID NO:117].

FIG. 16 presents a schematic representation of single, double and triple A domain avimer constructs.

FIG. 17 depicts (A) a schematic representation of the acceptor vector used in the construction of the avimer constructs and for CDR diversification, and (C) the nucleotide sequences for the vector represented in (A) [SEQ ID NO:118] (BsaI and KpnI restriction sites are bolded).

FIG. 18 depicts (A) the sequences of RSS flanked cassettes used to introduce sequence diversity into avimer sequences and corresponding amino acids, and (B) the CCA nucleotides changed to TGT introducing cysteines in two additional reading frames.

### DETAILED DESCRIPTION OF THE INVENTION

The present invention relates to an in vitro system for generating sequence, and thus structural, diversity in proteins. The system can be constructed using appropriately selected nucleic acid molecules that encode regions of a selected protein or proteins and recombination signal sequences (RSS). The selected protein(s) can be, for example, immunoglobulin (Ig) V, D, J and/or C regions, regions of a non-immunoglobulin (non-Ig) protein, or a combination of Ig regions and non-Ig regions. Assembly of such appropriately selected components and their introduction into suitable recombination-competent host cells allows for recombination between the RSS sequences and introduction of sequence and structural diversity into the protein(s).

### DEFINITIONS

Unless defined otherwise, all technical and scientific terms used herein have the same meaning as commonly understood by one of ordinary skill in the art to which this invention belongs.

“Naturally occurring,” as used herein with reference to an object, refers to the fact that the object can be found in nature. For example, an organism, or a polypeptide or polynucleotide sequence that is present in an organism that can be isolated from a source in nature and which has not been intentionally modified by man in the laboratory is naturally occurring.

The term “isolated,” as used herein with reference to a material, means that the material is removed from its original environment (for example, the natural environment if it is naturally occurring). For example, a naturally occurring polynucleotide or polypeptide present in a living animal is not isolated, but the same polynucleotide or polypeptide separated from some or all of the co-existing materials in the natural system, is isolated. Such polynucleotides could be part of a vector and/or such polynucleotides or polypeptides could be part of a composition, and still be isolated in that such vector or composition is not part of its natural environment.

The term “gene,” as used herein, refers to a segment of DNA involved in producing a polypeptide chain. The segment of DNA may include regions preceding and/or following the coding region, as well as intervening sequences (introns) between individual coding segments (exons), and may also include regulatory elements (for example, promoters, enhancers, repressor binding sites and the like).

The term “deletion” as used herein with reference to a polynucleotide, polypeptide or protein has its common meaning as understood by those familiar with the art and may refer to molecules that lack one or more of a portion of a sequence from either terminus or from a non-terminal region, relative to a corresponding full length molecule. For example, in certain embodiments, a deletion may be a deletion of between 1 and about 1500 contiguous nucleotide or amino acid residues from the full length sequence.

The term “expression vector,” as used herein, refers to a vehicle used in a recombinant expression system for the purpose of expressing a polynucleotide sequence constitutively or inducibly in a host cell, including prokaryotic, yeast, fungal, plant, insect or mammalian host cells, either in vitro or in vivo. The term includes both linear and circular expression systems. The term includes expression systems that remain episomal and expression systems that integrate into the host cell genome. The expression systems can have the ability to self-replicate or they may not (for example, they may drive only transient expression in a cell).

The term “tripartite reaction,” as used herein, refers to a recombination reaction that involves two pairs of RSSs (each 12 bp and 23 bp, or 23 bp and 12 bp). An example of a tripartite reaction is in vivo immunoglobulin heavy chain recombination, which joins the V, the D and the J gene segments. A tripartite reaction generates two independent coding junctions. Two sequential bipartite reactions can be considered to be a tripartite reaction in that a tripartite reaction may comprise two bipartite reactions occurring in the same substrate, usually (but not always) in close temporal time. The tripartite reaction can occur in the presence or absence of TdT.

As used herein, the term “about” refers to an approximately +/−10% variation from a given value. It is to be understood that such a variation is always included in any given value provided herein, whether or not it is specifically referred to.

The term “plurality” as used herein means more than one, for example, two or more, three or more, four or more, and the like.

### Immunoglobulins

Certain embodiments of the invention disclosed herein are based on the surprising discovery that an in vitro system for generating antibody diversity can be constructed using appropriately selected nucleic acid molecules that comprise immunoglobulin V, D, J and C region encoding polynucleotide sequences and recombination signal sequences (RSS). As described herein, by the assembly of such appropriately selected components and their introduction into suitable recombination-competent host cells, previously insurmountable challenges associated with the temporal regulation of V(D)J recombination can be overcome. Despite the identification over 18 years ago of the cis elements and trans factors involved in immunoglobulin gene rearrangement, as described above, an in vitro system for generating large antibody repertoires de novo has not been described prior to the present disclosure.

In particular, according to the present application it is disclosed for the first time that in an in vitro antibody gene recombination system, it is not required that an immunoglobulin D-J gene recombination event precedes a V-to-DJ recombination event in order to generate immunoglobulin sequence diversity.

In addition, the present invention provides, in certain embodiments, compositions and methods that overcome the presumed inefficiencies that would otherwise accompany generation of a productive in-frame V(D)J product using an in vitro system that lacks the regulatory mechanisms that are present in a developing lymphocyte. In the absence of these regulatory systems that exist in vivo there would be extreme biases in segment utilization.

In this regard and without wishing to be bound by theory, the presently disclosed embodiments successfully overcome problems associated with inefficiency in the generation by recombination of productive V-D-J junctions, and biases in the relative utilization of particular V, D and/or J gene segments, when cellular regulatory mechanisms, which govern the temporal steps of first mediating a D-J recombination event prior to a V-(D-J) recombination event, are not present. Such inefficiencies and biases arise due to the need for multiple recombination events having unequal probabilities to take place during immunoglobulin gene rearrangement (and during which intervening sequences that include unused coding regions are deleted) in order for certain V, D and J segments to be utilized, given the disparity in the number of V, D and J genes.

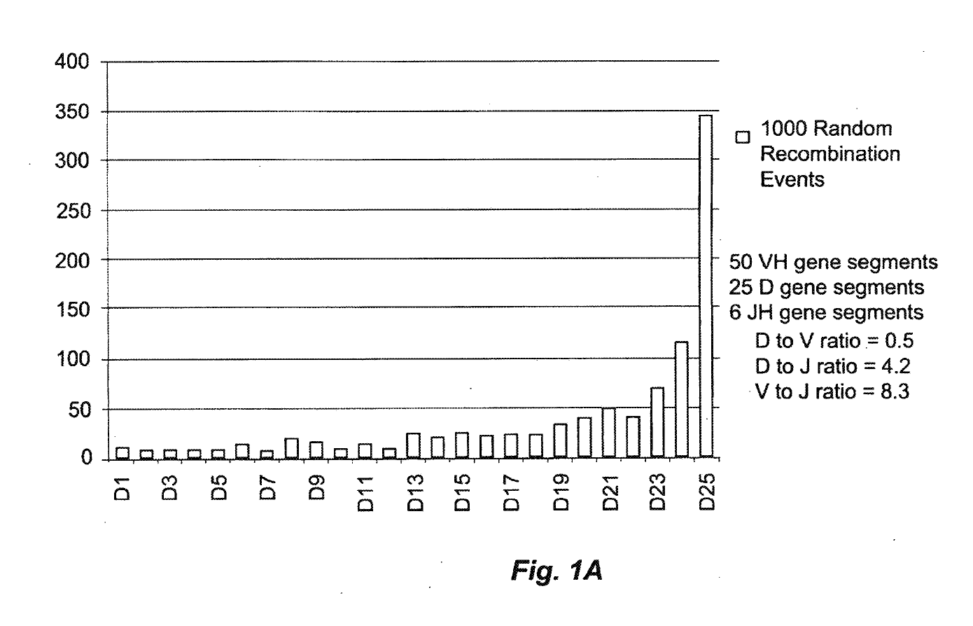

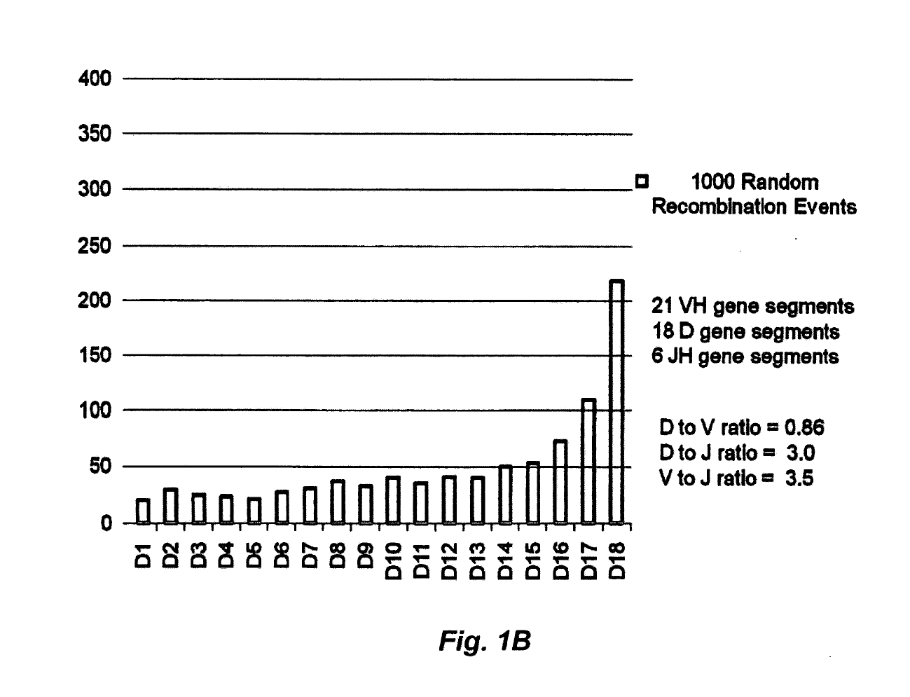

For example, the human Ig V_{H} locus comprises 51 functional V_{H}, 25 functional D and 6 functional J_{H} gene segments. As shown in FIG. 1A, 1,000 random V-D-J recombination events (according to a paradigm whereby random V-D events and random D-J events are queried for selection of a common D segment, and whereby equal efficiencies of recombination signal sequences are assumed) within a theoretical Ig V_{H} locus having 50 functional V_{H}, 25 functional D and 6 functional J_{H} gene segments, generate an output set having significant disparities in D segment utilization. Further inefficiencies are likely to result from non-productive recombination events. Inversional recombination events will also impact the efficiency of the reaction but do not have a significant impact on segment utilization since gene segment sequences are inverted and not lost. As shown in FIG. 1B, even by reducing the complexity of the theoretical Ig V_{H} locus to one having 21 functional V_{H}, 18 functional D and 6 functional J_{H} gene segments, gross disparities in D segment utilization persist.

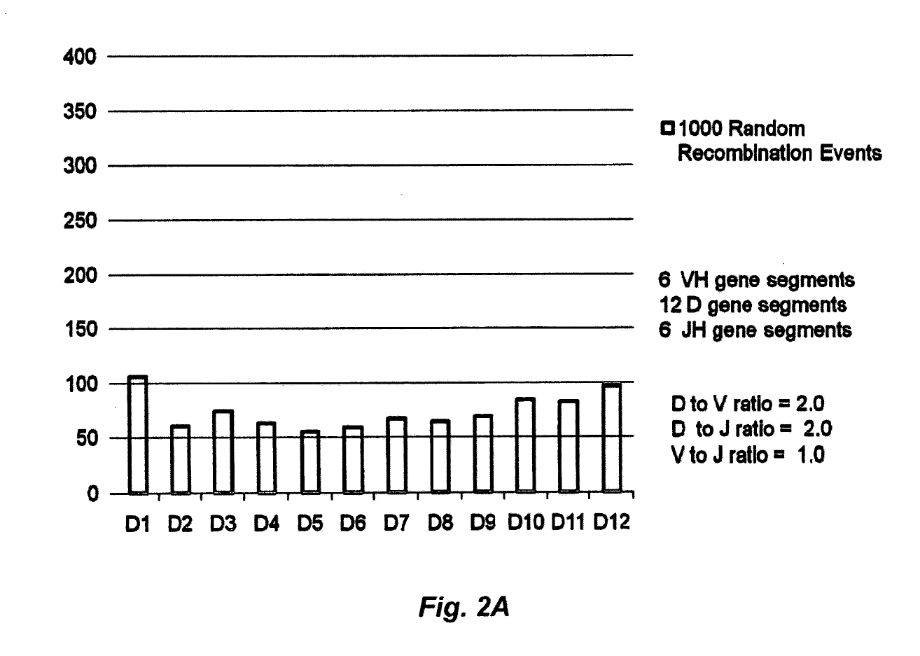

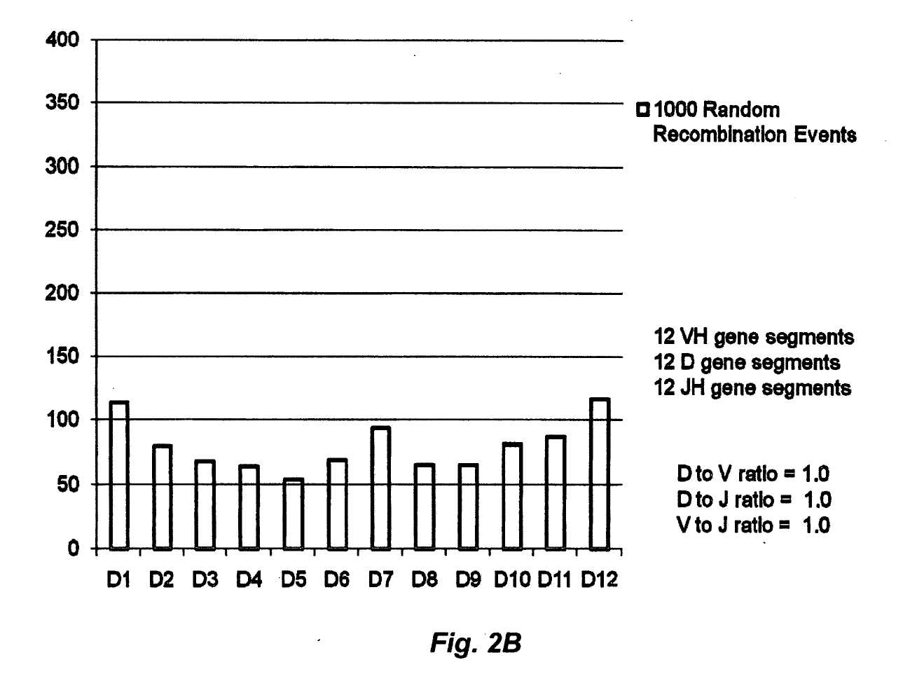

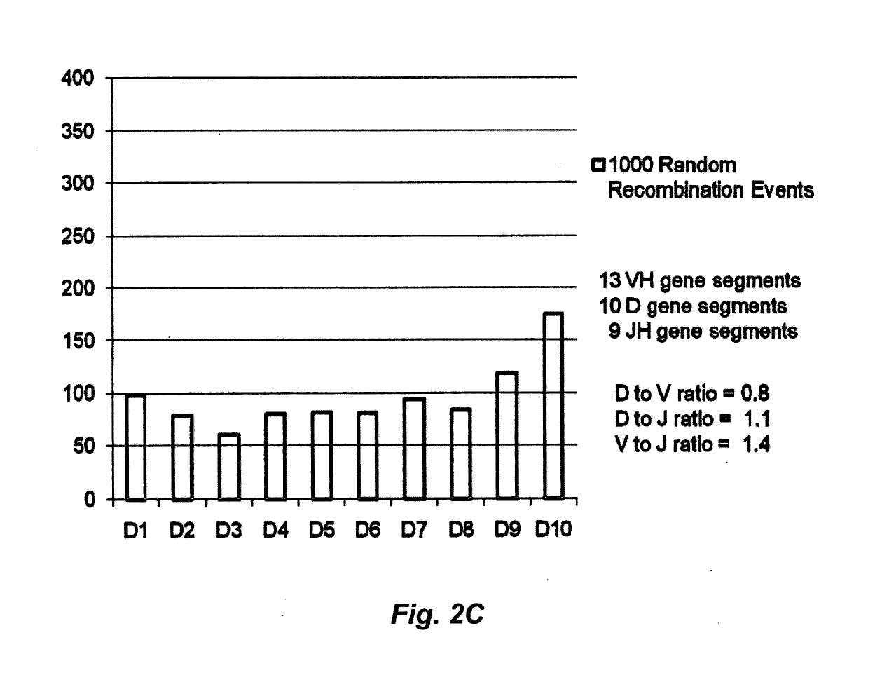

By contrast, according the present disclosure there are provided for the first time compositions and methods in which greater immunoglobulin structural diversity can be generated in vitro through selection of appropriate relative representation of the immunoglobulin gene elements to generate a highly diverse repertoire. As shown in FIG. 2, for example, such enhanced structural diversity is obtained when the ratio of V_{H} region genes to D segment genes is about 1:1 to 1:2 and the ratio of J_{H} segment genes to D segment genes is about 1:1 to 1:2, or when the ratio of V_{H} region genes to J_{H} segment genes is about 1:2 (V to J) to 2:1 (V to J), or when the combined number of V_{H} region genes together with J_{H} segment genes is not greater than the number of D segment genes when there is a plurality of D gene segments, or when 6, 7, 8, 9, 10, 11 or 12 D segment genes are present. A parameter that is described as being “about” a certain quantitative value typically may have a value that varies (i.e., may be greater than or less than) from the stated value by no more than 50%, and in preferred embodiments by no more than 40%, 30%, 25%, 20%, 15%, 10% or 5%. According to certain preferred embodiments as elaborated herein, the unexpected arrival at the present subject matter thus results from previously unappreciated significance of the gene segment usage biases that become apparent in vitro in the absence of the regulation normally imparted during recombination in vivo (as discussed supra), and of the importance of the relative ratios of the gene segments.

According to preferred embodiments disclosed herein, a nucleic acid composition for generating immunoglobulin structural diversity may be assembled from herein specified immunoglobulin gene elements, including naturally occurring and artificial sequences, using genetic engineering methodologies and molecular biology techniques with which those skilled in the art will be familiar. Useful immunoglobulin genetic elements for producing the compositions described herein include mammalian Ig heavy chain variable (V_{H}) and light chain variable (V_{L}) region genes, natural or artificial Ig diversity (D) segment genes, Ig heavy chain joining (J_{H}) and light chain joining (J_{L}) segment genes, and Ig locus recombination signal sequences (RSSs). Immunoglobulin variable (V) region genes are known in the art and include in their polypeptide-encoding sequences at least the polynucleotide coding sequence for one antibody complementarity determining region (CDR), for example, a first or a second CDR known as CDR1 or CDR2 according to conventional nomenclature with which those skilled in the art will be familiar, preferably coding sequence for two CDRs, for example, CDR1 and CDR2, and more preferably coding sequence for CDR1 and CDR2 and at least a portion (e.g., 1, 2, 3, 4, 5, 6, 7, 8, 9, 10, 11, 12, 13 or more amino acids) of CDR3, where it will be appreciated that typically one or more amino acids of CDR3 may be encoded at least in part by at least one nucleotide that is present in a D segment gene and/or in a J segment gene. (See, e.g., Lefranc M.-P., 1997 Immunology Today 18:509; Lefranc, 1999 The Immunologist, 7:132-13; Lefranc et al., 2003 Dev. Comp. Immunol. 27:55-77; Ruiz et al., 2002 Immunogenetics 53:857-883; Kaas et al., 2007 Current Bioinformatics 2:21-30; Kaas et al., 2004 Nucl. Acids. Res., 32:D208-D210.)

Immunoglobulin D segment genes are also known in the art and as provided herein may include coding regions for natural or non-naturally occurring D segments which coding regions comprise 1, 2, 3, 4, 5, 6, 7, 8, 9, 10, 11, 12, 13, 14, 15, 16, 17, 18, 19, 20, 21, 22, 23 or 24 nucleotides. Immunoglobulin J segment genes are also known in the art, for example, from immunoglobulin genes or cDNAs that have been sequenced, and typically comprise J segment-encoding regions of about 1-51 nucleotides.

As described herein, many such Ig gene sequences are therefore known in the art (e.g., Kabat et al., Sequences of Proteins of Immunological Interest, Edition: 5, 1992 DIANE Publishing, 1992, Darby, Pa., ISBN 094137565X, 9780941375658; Tomlinson et al., 1992 J Mol Biol 227:776; Milner et al., 1995 Ann N Y Acad Sci 764:50) and can be used in the several embodiments herein disclosed, including mammalian Ig gene sequences from human, mouse, rat, rabbit, canine, feline, equine, bovine, monkey, baboon, macaque, chimpanzee, gorilla, orangutan, camel, llama, alpaca and ovine genomes. Preferred embodiments relate to human Ig gene sequences but the invention is not intended to be so limited.

### Non-Immunoglobulin Proteins

Certain embodiments of the invention are based on the finding, illustrated herein, that the use of components of the antibody V(D)J recombination system can be expanded outside their natural role of mediating assembly of antibody gene segments to their use to modify a non-immunoglobulin (non-Ig) protein sequence.

Accordingly, certain embodiments of the invention relate to methods of generating sequence diversity in a known protein sequence by targeted introduction of two or more recombination signal sequences (RSSs) into the protein coding sequence and subsequent introduction of the modified protein coding sequence into a recombination-competent host cell, such as a host cell that is capable of expressing at least RAG-1, RAG-2 and terminal deoxynucleotidyl transferase (TdT), resulting in the generation and expression of a structurally diversifies variant protein. Some embodiments of the present invention also relate to polynucleotides comprising a nucleic acid sequence encoding one or more regions of a protein and comprising two or more pairs of RSSs, and compositions comprising same.

Certain embodiments of the present invention recognizes that the natural V(D)J reaction has inherent characteristics, specifically the imprecise junctions generated during the joining process, that make it useful as a general means to generate sequence diversity.

In accordance with certain embodiments of the present invention, the methods of generating sequence diversity may be applied to a wide variety of proteins for which a functional assay can be designed for screening. Certain embodiments of the invention employ a ligand-binding protein or region thereof in the described methods, wherein the ligand may be an antigen, another protein, a nucleic acid, a carbohydrate, a lipid, a metal, a vitamin or the like. In the context of the present invention, the term “ligand-binding protein” includes receptor-binding proteins. In some embodiments, the target protein is a ligand-binding protein, wherein the ligand is another protein, a nucleic acid, a carbohydrate, a lipid, a vitamin or a metal. Some embodiments employ a ligand-binding protein or region thereof, wherein the ligand is another protein. Certain embodiments employ a ligand-binding protein or region thereof, wherein the ligand is an antigen. Some embodiments employ a receptor-binding protein or region thereof.

Non-Ig proteins that may be employed in certain embodiments of the invention include naturally-occurring proteins and non-naturally occurring proteins. Naturally-occurring proteins may include human proteins and non-human proteins, for example, proteins from a non-human animal, a plant, or a micro-organism. In some embodiments, the non-Ig protein may be a ligand-binding protein. Examples of naturally-occurring ligand-binding proteins include, but are not limited to, biotin-binding proteins (such as avidin and streptavidin), lipid-binding proteins (such as beta-lactoglobulin, alpha1-microglobulin and plasma transthyretin), periplasmic binding proteins, lectins, serum albumins, phosphate binding proteins, sulphate binding proteins, immunophilins, metal-binding proteins, DNA-binding proteins, GTP-binding proteins (G-proteins), transporter proteins and receptor proteins (soluble and non-soluble). Non-limiting examples of metal-binding proteins include transferrin, ferritin and metallothionein. Non-limiting examples of DNA-binding proteins include histones, transcription factors, single-stranded DNA-binding proteins and helicases. Non-limiting examples of transporter and receptor proteins include, haemoglobin, cytochromes, G-protein coupled receptors, adrenalin receptors, acetylcholine receptors, histamine receptors, dopamine receptors, serotonin receptors, glutamate receptors, serotonin transporters, oestrogen receptors, Ca2+ channels, Na+ channels and Cl− channels. Non-limiting examples of soluble receptors include receptors for peptide hormones or cytokines, such as receptors for growth factors, lymphokines, monokines, interleukins, interferons, chemokines, colony-stimulating factors, hematopoietic factors, neurotrophic factors and differentiation-inhibiting factors.

Non-naturally occurring ligand-binding proteins include, for example, polypeptides that comprise one or more ligand-binding domains or fragments of naturally-occurring proteins capable of binding a ligand, such as fibronectin III domains (for example, FN3 and Adnectins™), the immunoglobulin binding domain of Staphylococcus aureus protein A (“affibodies”), src homology domains 2 and 3 (SH2 and SH3, respectively) and PDZ domains. Non-naturally occurring ligand-binding proteins also include artificial ligand-binding proteins such as designed ankyrin repeat proteins (“DARPins”), avimers and aptamers.

In certain embodiments, the methods are applied to proteins that comprise one or more loops, in which a loop can be defined as a region supported by a protein scaffold that can carry altered amino acids or sequence insertions without substantially compromising the structure of the scaffold, and wherein sequence diversity is introduced into one or more of the loops. In some embodiments, the methods are applied to proteins that comprise one or more surface-exposed loops, wherein one or more of the loops are targeted locations for introduction of sequence diversity. Examples of loop containing proteins are found within various categories of proteins described above and include, for example, loop presenting scaffold proteins.

It is to be understood that the methods of the present invention are equally applicable to protein fragments and that the term “protein” thus incorporates both the full length protein and fragments of the protein, for example, functional fragments, fragments comprising one or more domains, and the like. In certain embodiments, fragments include one or more deletions from either terminus of the protein or a deletion from a non-terminal region of the protein, for example, in some embodiments, deletions of between about 1 and about 500 contiguous amino acid residues. In some embodiments, the fragments may comprise a deletion of between about 1 and about 300 contiguous amino acid residues, for example, between 1 and about 250 contiguous amino acid residues, between 1 and about 200 contiguous amino acid residues, between 1 and about 150 contiguous amino acid residues, between 1 and about 100 contiguous amino acid residues, or between 1 and about 50 contiguous amino acid residues, including deletions of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 contiguous amino acid residues. In some embodiments, deletions of between 1-30, 31-40, 41-50, 51-60, 61-70, 71-80, 81-90, 91-100, 101-150, 151-200, 201-250 or 251-300 contiguous amino acid residues are contemplated.

### Other Genetic Elements

Other genetic elements that may be useful in certain herein disclosed embodiments include membrane anchor domain polypeptide encoding polynucleotide sequences and variants or fragments thereof (e.g., primary sequence variants or truncated products that retain 3D structural properties of the corresponding unmodified polypeptide, such as space-filling, charge distribution and/or hydrophobicity/hydrophilicity) that encode membrane anchor domain polypeptides that localize the polypeptides in which they are present to the surfaces of cells in which they are expressed.

Other genetic elements that may be useful in certain herein disclosed embodiments include specific protein-protein association domain encoding polynucleotide sequences and variants and fragments thereof (e.g., primary sequence variants or truncated products that retain 3D structural properties of the corresponding unmodified polypeptide, such as space-filling, charge distribution and/or hydrophobicity/hydrophilicity) that mediate specific protein-protein associations such as specific binding, as described herein.

Specific binding interactions such as a specific protein-protein association or a specific antibody-antigen binding interaction preferably includes a protein-protein binding event, or an antibody-antigen binding event, having an affinity constant, Kₐ, of greater than or equal to about 10⁴ M⁻¹, more preferably of greater than or equal to about 10⁵ M⁻¹, more preferably of greater than or equal to about 10⁶ M⁻¹, and still more preferably of greater than or equal to about 10⁷ M⁻¹. Affinities of specific binding partners including antibodies can be readily determined using conventional techniques, for example, those described by Scatchard et al. (Ann. N.Y. Acad. Sci. USA 51:660 (1949)), by Harlow et al., in Antibodies: A Laboratory Manual, Cold Spring Harbor Laboratory (1988), by Weir, Handbook of Experimental Immunology, 1986, Blackwell Scientific, Boston, by Scopes, Protein Purification: Principles and Practice, 1987 Springer-Verlag, New York, by surface plasmon resonance (BIAcore, Biosensor, Piscataway, N.J., see, e.g., Wolff et al., Cancer Res. 53:2560-2565 (1993)) or by other techniques known to the art.

As noted above, certain genetic elements that may be useful in presently disclosed embodiments include recombination signal sequences (RSSs), which are nucleic acid sequences that comprise a heptamer and a nonamer separated by a spacer of either 12 or 23 nucleotides, and that are specifically recognized in a complex recombination mechanism according to which a first RSS having a 12-nucleotide spacer recombines with a second RSS having a 23-nucleotide spacer. The orientation of the RSS determines if recombination results in a deletion or inversion of the intervening sequence.

As also described above, extensive investigations of RSS processes have led to an understanding of nucleotide positions within RSSs that cannot be varied without compromising RSS functional activity in genetic recombination mechanisms, and of other nucleotide positions within RSSs that can be varied to alter (e.g., increase or decrease in a statistically significant manner) the efficiency of RSS functional activity in genetic recombination mechanisms, and of other positions within RSSs that can be varied without having any significant effect on RSS functional activity in genetic recombination mechanisms (e.g., Ramsden et. al 1994; Akamatsu et. al. 1994 J Immunol 153:4520; Hesse et. al. 1989 Genes Dev 3:1053; Fanning et. al. 1996; Larijani et. al 1999; Nadel et. al. 1998 J Exp Med 187:1495; Lee et al. 2003 PLoS Biol 1:E1; Cowell et al. 2004 Immunol. Rev. 200:57).

According to the presently contemplated embodiments, an RSS may be any RSS that is known to the art, including sequence variants of known RSSs that comprise one or more nucleotide substitutions (e.g., about 1, 2, 3, 4, 5, 6, 7, 8, 9, 10, 11, 12, 13, 14, 15, 16, 17, 18 or more substitutions) relative to the known RSS sequence and which, by virtue of such substitutions, predictably have low efficiency (e.g., about 1% or less, relative to a high efficiency RSS), medium efficiency (e.g., about 10% to about 20%, relative to a high efficiency RSS) or high efficiency, including those variants for which one or more nucleotide substitutions relative to a known RSS sequence will have no significant effect on the recombination efficiency of the RSS (e.g., the success rate of the RSS in promoting formation of a recombination product, as known in the art and readily determined according to assays such as those disclosed in Hesse et al., 1989 Genes Dev 3:1053; Akamatsu et al., 1994 J Immunol 153:4520; Nadel et al., 1998 J Exp Med 187:1495; Lee et al., 2003 PLoS Biol 1:E1; Cowell et al., 2004 Immunol Rev 200:57).

Further according to the presently disclosed embodiments, it is to be understood that when, for instance, a first nucleic acid comprising a first RSS is described as being capable of functional recombination with a second RSS that is present in a second nucleic acid, such capability includes compliance with the 12/23 rule for RSS nucleotide spacers as described herein and known in the art, such that if the first RSS comprises a 12-nucleotide spacer then the second RSS will comprise a 23-nucleotide spacer, and similarly if the first RSS comprises a 23-nucleotide spacer then the second RSS will comprise a 12-nucleotide spacer.

Certain embodiments of the presently disclosed nucleic acid compositions comprise one or more of first, second, third and fourth isolated nucleic acids as described herein, where such nucleic acids may be separate molecules or may be joined into a single nucleic molecule, or may be present as two or three nucleic acid molecules, so long as the nucleic acid is capable of undergoing recombination events to form a recombined polynucleotide that encodes a polypeptide as recited. These nucleic acid compositions may comprise one or more RSSs which, as noted above, may be any RSS provided the 12/23 rule for RSS spacers is satisfied in any particular nucleic acid composition as a whole. The identities of particular RSSs may be specified by qualifying the RSS according to a particular genetic element with which it is associated in an isolated nucleic acid.

For example, where a nucleic acid composition comprises a first isolated nucleic acid that comprises one or a plurality of mammalian immunoglobulin heavy chain variable (V_{H}) region genes, each having a V_{H} encoding polynucleotide sequence and a RSS that is situated 3′ to the V_{H} encoding polynucleotide sequence, the RSS may be referred to as a “V_{H} region RSS” that is located 3′ to the V_{H} encoding sequence. As another example, where a nucleic acid composition comprises a second isolated nucleic acid that comprises one or a plurality of mammalian immunoglobulin heavy chain diversity (D) segment genes, each having a D segment encoding polynucleotide sequence and two RSSs, with the first RSS being situated 5′ to the D segment encoding sequence and the second RSS being situated 3′ to the D segment encoding sequence, the first RSS may be referred to as “a D segment upstream RSS” that is located 5′ to each D segment encoding sequence, and the second RSS may be referred to as “a D segment downstream RSS” that is located 3′ to each D segment encoding sequence. The skilled person will accordingly appreciate what is meant by other similarly specified RSSs, including, for example, an RSS that is “a J_{H} segment RSS” that is located 5′ to a J_{H} segment encoding polynucleotide sequence, another RSS that is “a V_{L} region RSS” that is located 3′ to a V_{L} region encoding polynucleotide sequence, and another RSS that is “a J_{L} segment RSS” that is located 5′ to a J_{L} segment encoding polynucleotide sequence.

Examples of RSS sequences known to the art, including their characterization as high, medium or low efficiency RSSs, are presented in Table 1.

TABLE 1
EXEMPLARY RECOMBINATION SIGNAL SEQUENCES

| | | | | | | | | |
|---|---|---|---|---|---|---|---|---|
| | | | Seq. | | | | Seq | |
| heptamer | spacer | nonamer | ID | heptamer | spacer | nonamer | Id | |
| H12 | S12 | N12 | No. | H23 | S23 | N23 | No: | * |

Part I. Efficiency: HIGH

| | | | | | | | | |
|---|---|---|---|---|---|---|---|---|
| CACAGTG | ATACAG | ACAAAAAC | 29 | CACAGTG | GTAGTACTCCACTGTCTGGC | ACAAAAACC | 30 | 4 |
| | ACCTTA | C | | | TGT | | | |
| CACAGTG | CTACAG | ACAAAAAC | 31 | CACAGTG | GTAGTACTCCACTGTCTGGC | ACAAAAACC | 32 | 3 |
| | ACTGGA | C | | | TGT | | | |
| CACAGTG | CTCCAG | ACAAAAAC | 33 | CACAGTG | GTAGTACTCCACTGTCTGGG | ACAAAAACC | 34 | 1 |
| | GGCTGA | C | | | TGT | | | |
| CACAGTG | CTACAG | ACAAAAAC | 35 | CACAGTG | TTGCAACCACATCCTGAGTG | ACAAAAACC | 36 | 2 |
| | ACTGGA | C | | | TGT | | | |
| CACAGTG | CTACAG | ACAAAAAC | 37 | CACAGTG | GTAGTACTCCACTGTCTGGC | ACAAAAACC | 38 | 2 |
| | ACTGGA | C | | | TGT | | | |
| CACAGTG | CTACAG | ACAAAAAC | 39 | CACAGTG | ACGGAGATAAAGGAGGAAG | ACAAAAACC | 40 | 2 |
| | ACTGGA | C | | | CAGG | | | |
| CACAGTG | GTACAG | ACAGAAAC | 41 | CACAGTG | GCCGGGCCCCGCGGCCCG | ACAAAAACC | 42 | 5 |
| | ACCAAT | C | | | GCGGC | | | |

Part II. Efficiency: MEDIUM (~10-20% of High)

| | | | | | | | | |
|---|---|---|---|---|---|---|---|---|
| CACGGTG | CTACAG | ACAAAAAC | 43 | CACAGTG | GTAGTACTCCACTGTCTGGC | ACAAAAACC | 44 | 3 |
| | ACTGGA | C | | | TGT | | | |
| CACAATG | CTACAG | ACAAAAAC | 45 | CACAGTG | GTAGTACTCCACTGTCTGGC | ACAAAAACC | 46 | 3 |
| | ACTGGA | C | | | TGT | | | |
| CACAGCG | CTACAG | ACAAAAAC | 47 | CACAGTG | GTAGTACTCCACTGTCTGGC | ACAAAAACC | 48 | 3 |
| | ACTGGA | C | | | TGT | | | |
| CACAGTG | CTACAG | ACAAAAAC | 49 | CACAATG | GTAGTACTCCACTGTCTGGC | ACAAAAACC | 50 | 3 |
| | ACTGGA | C | | | TGT | | | |
| CACAGTG | CTACAG | ACAAAAAC | 51 | CACAGCG | GTAGTACTCCACTGTCTGGC | ACAAAAACC | 52 | 3 |
| | ACTGGA | C | | | TGT | | | |
| CACAGTG | CTACAG | ACAAAAAC | 53 | CACAGTA | GTAGTACTCCACTGTCTGGC | ACAAAAACC | 54 | 3 |
| | ACTGGA | C | | | TGT | | | |
| CACAGTG | CTACAG | ACAAAAAC | 55 | CACAGTG | GTAGTACTCCACTGTCTGGC | ACAATAACC | 56 | 3 |
| | ACTGGA | C | | | TGT | | | |
| CACAGTG | CTACAG | ACAAAAAC | 57 | CACAGTG | GTAGTACTCCACTGTCTGGC | ACAAGAACC | 58 | 3 |
| | ACTGGA | C | | | TGT | | | |
| CACAGTG | CTACAG | ACAAAAAC | 59 | CACAGTG | GTAGTACTCCACTGTCTGGC | ACACGAAC | 60 | 3 |
| | ACTGGA | C | | | TGT | C | | |
| CACAGTG | CTACAG | CAAAAACC | 61 | CACAGTG | GTAGTACTCCACTGTCTGGC | ACAAAAACC | 62 | 3 |
| | ACTGGA | C | | | TGT | | | |
| CACAGTG | CTACAG | ACAAAAAC | 63 | CACAGTG | GTAGTACTCCACTGTCTGGC | ACACGAAC | 64 | 3 |
| | ACTGGA | C | | | TGT | C | | |
| CACAATG | CTACAG | ACAAAAAC | 65 | CACAATG | GTAGTACTCCACTGTCTGGC | ACAAAAACC | 66 | 3 |
| | ACTGGA | C | | | TGT | | | |
| CACAGCG | CTACAG | ACAAAAAC | 67 | CACAGCG | GTAGTACTCCACTGTCTGGC | ACAAAAACC | 68 | 3 |
| | ACTGGA | C | | | TGT | | | |

Part III. Efficiency: LOW (~1% or less of High)

| | | | | | | | | |
|---|---|---|---|---|---|---|---|---|
| TACAGTG | CTACAG | ACAAAAAC | 69 | CACAGTA | GTAGTACTCCACTGTCTGGC | ACAAAAACC | 70 | 3 |
| | ACTGGA | C | | | TGT | | | |
| GACAGTG | CTACAG | ACAAAAAC | 71 | CACAGTG | GTAGTACTCCACTGTCTGGC | ACAAAAACC | 72 | 3 |
| | ACTGGA | C | | | TGT | | | |
| CATAGTG | CTACAG | ACAAAAAC | 73 | CACAATG | GTAGTACTCCACTGTCTGGC | ACAAAAACC | 74 | 3 |
| | ACTGGA | C | | | TGT | | | |
| CACAATG | CTACAG | ACAAAAAC | 75 | CATAGTG | GTAGTACTCCACTGTCTGGC | ACAAAAACC | 76 | 3 |
| | ACTGGA | C | | | TGT | | | |
| CACAGTG | CTACAG | ACAAAAAC | 77 | CACAGTG | GTAGTACTCCACTGTCTGGC | TGTCTCTGA | 78 | 3 |
| | ACTGGA | C | | | TGT | | | |
| CAGAGTG | CTCCAG | ACAAAAAC | 79 | CACAGTG | GTAGTACTCCACTGTCTGGG | ACAAAAACC | 80 | 1 |
| | GGCTGA | C | | | TGT | | | |
| CACAGTG | CTCCAG | AAAAAAAC | 81 | CACAGTG | GTAGTACTCCACTGTCTGGG | ACAAAAACC | 82 | 1 |
| | GGCTGA | C | | | TGT | | | |
| CTCAGTG | CTCCAG | ACAAAAAC | 83 | CACAGTG | GTAGTACTCCACTGTCTGGG | ACAAAAACC | 84 | 1 |
| | GGCTGA | C | | | TGT | | | |

*(1) Akamatsu et al. 1994; (2) Cowell et al. 2004; (3) Hesse et al. 1989; (4) Lee et al. 2003; (5) Nadel et al. 1998.

### Positioning the RSS Sequences in Ig Coding Sequences

Certain preferred embodiments contemplate construction of nucleic acid compositions for generating immunoglobulin structural diversity as provided herein whereby selection of RSSs of known efficiencies at prescribed positions may advantageously counteract biases in particular immunoglobulin gene utilization that would otherwise result from the relative locations of the several Ig genetic elements. More specifically, and without wishing to be bound by theory, the nucleic acid compositions disclosed herein are envisioned as comprising, in a 5′ to 3′ orientation according to molecular biology conventions for designating directionality to a DNA coding strand:
(a) one or a plurality of Ig V region genes, each having (i) an Ig V region encoding polynucleotide sequence and (ii) a V region RSS that is located 3′ to the V region encoding polynucleotide;
(b) one or a plurality of Ig D segment genes, each having (i) a D segment encoding polynucleotide sequence, (ii) a D segment upstream RSS that is located 5′ to the D segment encoding polynucleotide, and (iii) a D segment downstream RSS that is located 3′ to the D segment encoding polynucleotide; and
(c) one or a plurality of Ig J segment genes, each having (i) a J segment encoding polynucleotide sequence and (ii) a J segment RSS that is located 5′ to the J segment encoding polynucleotide.

According to such a configuration, it will be appreciated that in the course, simultaneously or sequentially and in either order, of functional recombination of the V region RSS with the D segment upstream RSS, and functional recombination of the D segment downstream RSS with the J segment RSS, unused intervening V, D and J genes are deleted such that if the selection of V, D and J genes is random, the frequency of usage of particular genes will be biased.

For example, V region genes situated closer to the 5′ end of the construct are likely to be overused in productive RSS-RSS recombination events, because they have a lower probability of being deleted during V-D recombination, while V region genes situated closer to the 3′ end of (a) are likely to be underused given the higher probability they will be deleted during recombination. Similarly, D segment genes situated at or near the 5′ end of (b) are likely to be underused, while those situated at or near the 3′ end of (b) are more likely to survive deletion events accompanying recombinase-mediated DNA cleavage and subsequent repair, and so would be overused in productive recombination events.

As provided herein, enhanced generation of immunoglobulin structural diversity in the present artificial system is accomplished through efficient and relatively unbiased utilization of Ig V, D and J genetic elements, including by designing nucleic acid constructs that have defined relative ratios of V, D and J genes and/or restricted number of D segment genes and/or by strategic positioning of RSSs of predefined efficiencies.

Accordingly, in certain embodiments there is provided a nucleic acid composition for generating Ig structural diversity that comprises one or a plurality of Ig V region genes, Ig D segment genes, and Ig J segment genes as described herein, and optionally further comprising a polynucleotide encoding a membrane anchor domain polypeptide and/or a polynucleotide encoding a specific protein-protein association domain, in which (a) the V region genes and the D segment genes are present at a ratio of about 1:1 to 1:2, and the J segment genes and the mammalian D segment genes are present at a ratio of about 1:1 to 1:2; or in which (b) the V region genes and the J segment genes are present at a ratio of about 1:2 (V to J) to 2:1(V to J); or in which (c) the V region genes, together with the J segment genes, are not greater in number than the D segment genes; or in which (d) there are 6, 7, 8, 9, 10, 11 or 12 D segment genes.

In certain further embodiments, (a) 12-50 contiguous V region genes (in preferred embodiments V_{H} region genes) are present of which about 10% to about 30% of said V region genes are contiguous with a 5′-most located V region gene and each V region gene comprises a V region (preferably a V_{H} region) RSS of low or medium RSS efficiency, and of which about 70% to about 90% of said V region genes are contiguous with a 3′-most located V region gene and each comprises a V region RSS of high RSS efficiency; and (b) a plurality of contiguous D segment genes are present of which (i) about 80% to about 90% of said D segment genes are contiguous with a 5′-most located D segment gene and each comprises a D segment upstream RSS of high RSS efficiency and a D segment downstream RSS of high RSS efficiency, and (ii) about 10% to about 20% of said D segment genes are contiguous with a 3′-most located D segment gene and each comprises a D segment upstream RSS of low or medium RSS efficiency and a D segment downstream RSS of low or medium RSS efficiency, wherein the plurality of V region genes, together with the one or a plurality of J segment genes, are not greater in number than said plurality of D segment genes.

It will be understood by those familiar with the art that by convention and due to nucleic acid 5′-to-3′ polarity, a nucleic acid coding strand comprises an upstream or 5′ end (or 5′ terminus) and a downstream or 3′ end (or 3′ terminus) such that in the linear polymer containing a plurality of linked and tandemly, consecutively and/or sequentially arrayed (e.g., contiguous) genes, a single gene (e.g., of a designated class, such as a V region gene) may be situated closer to the 5′ terminus than all others (e.g., the “5′-most located” gene) and a different single gene (e.g., of the designated class) may be situated closer to the 3′ terminus than all the others (e.g., the “3′-most located” gene). Hence, distribution of RSSs having specified recombination efficiencies amongst the plurality of contiguous genes in the nucleic acid molecule will vary according to the number of genes that are used in a particular construct, in order for a specified percentage of such genes to comprise a specified RSS type. Additionally and as provided herein according to certain preferred embodiments such RSS distributions will accordingly confer gene utilizations that are about equal, thereby advantageously providing compositions for generating increased Ig structural diversity.

In related but distinct embodiments, there is accordingly provided a nucleic acid composition for generating Ig structural diversity that comprises one or a plurality of Ig V region genes, Ig D segment genes, and Ig J segment genes as described above, and that is characterized by one or more of (a) 12-50 contiguous V (preferably V_{H}) region genes are present of which about 10% to about 30% are contiguous with a 5′-most located V region gene and each V region gene comprises a V region RSS of low or medium RSS efficiency; (b) 12-50 contiguous V (preferably V_{H}) region genes are present of which about 70% to about 90% are contiguous with a 3′-most located V region gene and each V region gene comprises a V region RSS of high RSS efficiency; (c) a plurality of contiguous D segment genes are present of which about 80% to about 90% are contiguous with a 5′-most located D segment gene and each D segment gene comprises a D segment upstream RSS of high RSS efficiency and a D segment downstream RSS of high RSS efficiency; and (d) a plurality of contiguous D segment genes are present of which about 10% to about 20% are contiguous with a 3′-most located D segment gene and each comprises a D segment upstream RSS of low or medium RSS efficiency and a D segment downstream RSS of low or medium RSS efficiency.

As disclosed herein according to certain embodiments there are provided nucleic acid compositions for generating immunoglobulin structural diversity by including, for example by way of illustration and not limitation in a composition that contains immunoglobulin light chain-encoding sequences (e.g., V_{L} and J_{L}), an immunoglobulin diversity (D) segment gene, which may in certain related embodiments comprise a naturally occurring D segment encoding sequence (e.g., Corbett et al., 1997 J Mol Biol 270:587; NCBI locus NG_{—}001019; vbase, 1997 MRC Centre for Protein Engineering). In certain distinct but related embodiments, however, a nucleic acid composition as provided herein, for instance and without limitation, an Ig light-chain or light-chain fusion protein encoding nucleic acid composition, may comprise an artificial D segment gene that may comprise a non-naturally occurring sequence encoding an artificial D segment and that is positioned to be recombined between V_{L} and J_{L}, and which may comprise a nucleotide sequence representing a subset or combination of sequences found in any human D segment gene including a single nucleotide, a dinucleotide or a fusion of complete or partial human D segment gene sequences, but which in preferred embodiments is not generally recognized as a conventional human D segment gene. Such an artificial D segment encoding sequence of 1, 2, 3, 4, 5, 6, 7, 8, 9, 10, 11, 12, 13, 14, 15, 16, 17, 18, 19, 20, 21, 22, 23 or 24 nucleotides is contemplated. Accordingly, a D segment encoding sequence may include a single nucleotide, or any dinucleotide, or any combination of two or more fused D segment encoding polynucleotide sequences from two or more distinct, recognized immunoglobulin D segment genes that occur naturally in a genome, preferably the human genome. Non-limiting examples of D segment encoding polynucleotide sequences are presented in Table 2.

TABLE 2
EXEMPLARY D SEGMENT ENCODING SEQUENCES

| | | | SEQ |
|---|---|---|---|
| | | | ID |
| D | # | Nucleotide Sequence | NO: |
| D1 | 1-1 | GGTACAACTGGAACGAC | 85 |
| | 1-7 | GGTATAACTGGAACTAC | 86 |
| | 1-20 | GGTATAACTGGAACGAC | 87 |
| | 1-26 | GGTATAGTGGGAGCTACTAC | 88 |
| D2 | 2-2 | AGGATATTGTAGTAGTACCAGCTGCTATACC | 89 |
| | 2-8 | AGGATATTGTACTAATGGTGTATGCTATACC | 90 |
| | 2-15 | AGGATATTGTAGTGGTGGTAGCTGCTACTCC | 91 |
| | 2-21 | AGCATATTGTGGTGGTGACTGCTATTCC | 92 |
| D3 | 3-3 | GTATTACGATTTTTGGAGTGGTTATTATACC | 93 |
| | 3-9 | GTATTACGATATTTTGACTGGTTATTATAAC | 94 |
| | 3-10 | GTATTACTATGGTTCGGGGAGTTATTATAAC | 95 |
| | 3-16 | GTATTATGATTACGTTTGGGGGAGTTATCGTTATACC | 96 |
| | 3-22 | GTATTACTATGATAGTAGTGGTTATTACTAC | 97 |
| D4 | 4-4 | TGACTACAGTAACTAC | 98 |
| | 4-11 | TGACTACAGTAACTAC | 99 |
| | 4-17 | TGACTACGGTGACTAC | 100 |
| | 4-23 | TGACTACGGTGGTAACTCC | 101 |
| D5 | 5-5 | GTGGATACAGCTATGGTTAC | 102 |
| | 5-12 | GTGGATATAGTGGCTACGATTAC | 103 |
| | 5-18 | GTGGATACAGCTATGGTTAC | 104 |
| | 5-24 | GTAGAGATGGCTACAATTAC | 105 |
| D6 | 6-6 | GAGTATAGCAGCTCGTCC | 106 |
| | 6-13 | GGGTATAGCAGCAGCTGGTAC | 107 |
| | 6-19 | GGGTATAGCAGTGGCTGGTAC | 108 |
| D7 | 7-27 | CTAACTGGGGA | 109 |

In certain embodiments a D segment gene may therefore be provided on immunoglobulin light chain diversity generating constructs, as described in detail, for instance, in Example 2. The inclusion of a D segment gene converts an otherwise bimolecular reaction system into a tripartite system. Because of the 12/23 pairing rule (discussed supra), in an exemplary bimolecular system all the V segments may be adjacent to RSSs (i.e., V region RSSs) having spacers of a first common size (e.g., utilizing either 12 or 23 nucleotides) and the J segments are all adjacent to RSSs (i.e., J segment RSSs) having spacers of a second common size that is not the same as the first common size used in V region RSS spacers. In other words, if the V region RSSs contain 23-nucleotide spacers then the J segment RSSs would contain 12-nucleotide spacers, and vice versa. This configuration directs V to J recombination, but without the regulation found in vivo it would continue to consume Ig gene segments until either only a single V or J gene segment remains, or until the recombinase is turned off by cellular mechanisms. In the absence of being able to turn off the recombinase in a specific cell that has completed recombination as is accomplished in vivo, continuing recombination would result in the vast underrepresentation of proximal V-J segments and would favor usage of the distal segments. In a tripartite system, the V and J segments would both use RSSs having the same spacer sizes (i.e., V region RSSs and J segment RSSs would have the same spacer size, being either 12- or 23-nucleotides) and the D segment gene RSSs (i.e., the D segment upstream RSS and the D segment downstream RSS) would each use the complementary RSS signal size (i.e., 23 nucleotides if V region RSSs and J segment RSSs use 12-nucleotide spacers, and 12 nucleotides if V region RSSs and J segment RSSs use 23-nucleotide spacers). In this exemplary configuration, because the V region RSSs and J segment RSSs have spacers of the same size, the 12/23 rule prevents them from recombining directly. Instead recombination proceeds through a D segment gene that comprises a D segment upstream RSS and a D segment downstream RSS having spacers of the same size. In certain related embodiments and without wishing to be bound by theory, it is contemplated therefore that limiting the number of D segment genes may limit the number of rounds of recombination that a particular Ig diversity-generating nucleic acid composition can undergo; recombination stops when there is only a single D segment remaining and all D segment RSSs have been utilized. In another related embodiment in which the Ig diversity-generating nucleic acid composition comprises one D segment gene, V-D recombination can occur only once via functional recombination of the D segment upstream RSS with the V region RSS, and D-J recombination can occur only once via functional recombination of the D segment downstream RSS with the J segment RSS, thus reducing biases in gene segment utilization.

As the D segment is found naturally in heavy chains and not light chains, these and related embodiments also contemplate unprecedented expansion of the immunoglobulin light chain variable region repertoire, by providing the D segment as an additional combinatorial source of structural diversity through V-D-J recombination events as described herein.

### Positioning the RSS Sequences in Non-Ig Coding Sequences

As noted above, in certain embodiments, complementary pairs of RSSs are introduced into the coding sequence for a non-Ig protein, in which the first RSS of the pair is capable of functional recombination with the second RSS of the pair. In accordance with these embodiments, the two RSSs of the complementary pair are separated by an intervening sequence of about 100 bp or more in length. The nucleotide sequence of the intervening sequence is not critical to the invention and may be comprised of a sequence heterologous to the coding sequence or it may be comprised of part of the coding sequence. For example, in certain embodiments, the complementary pair of RSSs are introduced individually into the coding sequence such that part of the coding sequence forms the intervening sequence. In other embodiments, the complementary pair of RSSs is introduced together with a heterologous intervening sequence into the coding sequence as a “cassette.” The nucleotide sequence of the intervening sequence can accommodate a wide variety of sequences, including for example some selectable markers, some promoters and other regulatory elements such as polyadenylation signals, but preferably does not include insulator like elements as exemplified by cHS4 and AAV1.

Regardless of the composition of the intervening sequence, it is preferably selected to be at least 100 bp in length, for example, at least 110 bp, at least 120 bp, at least 130 bp, at least 140 bp, at least 150 bp, but may range up to several kilobases in size, for example up to about 5 kb. One skilled in the art will understand that the exact upper limit for the intervening sequence will be dictated by the limitation of the vector system used. In certain embodiments, the intervening sequence is selected to be between about 100 bp and 5 kb, for example, between about 150 bp and 5 kb, between about 180 bp and 5 kb, between about 180 bp and 4 kb, between about 180 bp and 3 kb or between about 180 bp and 2 kb. In some embodiments, the intervening sequence is selected to be between about 100 bp and 1.5 kb, for example, between about 110 bp and 1.5 kb, between about 120 bp and 1.7 kb, between about 130 bp and 1.6 kb, between 140 bp and 1.5 kb, or between 150 bp and 1.5 kb. In some embodiments, the intervening sequence is selected to be between about 180 bp and 1.9 kb, for example, between about 180 bp and 1.8 kb, between about 180 bp and 1.7 kb, between about 180 bp and 1.6 kb, or between 180 bp and 1.5 kb. Other exemplary embodiments include intervening sequences of between about 190 bp and 1.5 kb, between about 200 bp and 1.5 kb, between about 210 bp and 1.5 kb, between about 220 bp and 1.5 kb, between about 230 bp and 1.5 kb, between about 240 bp and 1.5 kb, and between about 250 bp and 1.5 kb.

In certain embodiments, two or more complementary pairs of RSSs are introduced into the coding sequence in order to generate sequence diversity at more than one targeted location in the protein.

The RSSs can be introduced into the polynucleotide by standard genetic engineering techniques such as those described in Molecular Cloning: A Laboratory Manual (Third Edition) (Sambrook, et al., 2001, Cold Spring Harbour Laboratory Press, NY) and Current Protocols in Molecular Biology (Ausubel et al. (Ed.), 1987 & Updates, J. Wiley & Sons, Inc., Hoboken, N.J.).

Among the several embodiments described herein, there are also provided the means for generating structurally diverse gene libraries, including recombined genes encoding antibodies, non-Ig proteins or mixed Ig and non-Ig proteins having membrane anchor domains that permit their display on the surfaces of host cells expressing such genes. Advantages associated with cell surface expression, as distinct from secreted forms, of structurally diverse proteins as described herein, will be readily appreciated by persons familiar with the art in view of the present disclosure, for example, to facilitate the identification and/or selection of cells containing a particular rearranged gene, such as a cell expressing an antibody or antigen-binding protein having a desired antigen specificity, or a non-Ig protein having a desired activity.

In addition, certain preferred embodiments include the use of host cells that are capable of immunoglobulin gene rearrangement, but that may usefully be expanded in number without gene rearrangement taking place. In certain particularly preferred embodiments, such host cells are capable of expressing recombination control elements that mediate gene rearrangement events, but the expression of control elements is regulated in such a manner as to permit expansion of the host cell population prior to permitting the V-D-J gene rearrangement which generates sequence diversity.

As also described elsewhere herein, recombination control elements include the RAG-1, and RAG-2 genes and their respective gene products, for which defined roles in regulating immunoglobulin gene rearrangement/recombination events have been biochemically defined. Preferably such recombination control elements are operably linked to the nucleic acid compositions that, as described herein, comprise immunoglobulin structural domain-encoding polynucleotide sequences and recombination signal sequences (RSSs) and/or non-Ig protein encoding polynucleotide sequences. According to certain such embodiments a nucleic acid composition for generating protein structural diversity as provided herein is under control of an operably linked recombination control element when one, two or more recombination events that the nucleic acid composition undergoes to form a recombined polynucleotide that encodes a polypeptide or fusion protein are mediated by the recombination control element. The recombination control element may be inducible, for example, through regulation of its expression by a promoter such as a tightly regulated promoter.

For example and in certain preferred embodiments, a host cell that comprises a nucleic acid composition for generating protein structural diversity as provided herein, and that also comprises an operably linked inducible recombination control element that controls one or more recombination events which give rise to a productive protein encoding polynucleotide, may contain the chromosomally integrated nucleic acid composition under conditions wherein at least one component of the recombination control element (e.g., RAG-1 or RAG-2) is not constitutively (productively, e.g., at functionally relevant levels) expressed, but may be expressed upon exposure of the host cell to an inducer.

Such a host cell may advantageously be expanded to obtain a population of host cells bearing the chromosomally integrated nucleic acid composition, such that the expanded population can be induced with the inducer to obtain a population of cells each expressing a structurally diverse protein subsequent to two or more recombination events to form a recombined polynucleotide that encodes the protein, where such recombination events are mediated by recombination control elements the expression of which is induced by the inducer. This important feature of these and related preferred embodiments allows recombination to occur subsequent to expansion of the host cell population. According to non-limiting theory, such preferred embodiments (in which gene recombination takes place only after expansion of a host cell population) offer particular advantages associated with increasing the opportunities for different structurally diverse proteins to result from random recombination events in a large number of distinct cells that have chromosomally integrated the herein disclosed nucleic acid compositions for generating protein structural diversity. Further according to non-limiting theory, absent such an opportunity to first expand the host cell population, an Ig gene recombination-competent cell having a chromosomally integrated nucleic acid composition for generating protein structural diversity would be able to complete recombination soon after subcloning, such that only a limited number of different proteins would have been generated.

Certain related embodiments advantageously provide non-naturally occurring immunoglobulin fusion proteins that usefully feature immunoglobulin heavy chains having a membrane anchor domain polypeptide, and/or recombination-mediated assembly of functional immunoglobulin light chains having either or both of (i) a heavy chain diversity (D) segment (including an artificial D segment as described herein) and (ii) a specific protein-protein association domain or a lipid raft-associating polypeptide domain, where such modified immunoglobulin structures may facilitate generation of large antibody repertoires and identification of cells expressing an immunoglobulin or immunoglobulin-like molecule having a desired V region. Some embodiments relate to non-Ig protein fusions or mixed Ig and non-Ig protein fusions fused to a membrane anchor domain polypeptide, a specific protein-protein association domain or a lipid raft-associating polypeptide domain. Examples of specific protein-protein association domains include, but are not limited to, all or a protein-protein associating portion of a mammalian immunoglobulin C_{L} chain, or an RGD-containing polypeptide that is capable of integrin binding, or a heterodimer-promoting polypeptide domain, or other such domains as described herein and known in the art. Such fusion proteins may facilitate the generation of large libraries of sequence diversified proteins.

Hence, according to certain embodiments disclosed herein there are provided fusion polypeptides and proteins that localize to the cell surface by virtue of having naturally present or artificially introduced structural features that direct the fusion protein to the cell surface (e.g., Nelson et al. 2001 Trends Cell Biol. 11:483; Ammon et al., 2002 Arch. Physiol. Biochem. 110:137; Kasai et al., 2001 J. Cell Sci. 114:3115; Watson et al., 2001 Am. J. Physiol. Cell Physiol. 281:C215; Chatterjee et al., 200 J. Biol. Chem. 275:24013) including by way of illustration and not limitation, secretory signal sequences, leader sequences, plasma membrane anchor domain polypeptides such as hydrophobic transmembrane domains (e.g., Heuck et al., 2002 Cell Biochem. Biophys. 36:89; Sadlish et al., 2002 Biochem J. 364:777; Phoenix et al., 2002 Mol. Membr. Biol. 19:1; Minke et al., 2002 Physiol. Rev. 82:429) or glycosylphosphatidylinositol attachment sites (“glypiation” sites, e.g., Chatterjee et al., 2001 Cell Mol. Life. Sci. 58:1969; Hooper, 2001 Proteomics 1:748; Spiro, 2002 Glycobiol. 12:43 R), cell surface receptor binding domains, extracellular matrix binding domains, or any other structural feature that causes the fusion protein to localize to the cell surface.

Particularly preferred are fusion proteins that comprise a plasma membrane anchor domain, which may include a transmembrane polypeptide domain typically comprising a membrane spanning domain (e.g., an α-helical domain) which includes a hydrophobic region capable of energetically favorable interaction with the phospholipid fatty acyl tails that form the interior of the plasma membrane bilayer, or which may include a membrane-inserting domain polypeptide typically comprising a membrane-inserting domain which includes a hydrophobic region capable of energetically favorable interaction with the phospholipid fatty acyl tails that form the interior of the plasma membrane bilayer (e.g., outer leaflet phospholipids) but that may not span the entire membrane. Such features are well known to those of ordinary skill in the art, who will further be familiar with methods for introducing nucleic acid sequences encoding these features into the subject expression constructs by genetic engineering, and with routine testing of such constructs to verify cell surface localization of the product. Well known examples of transmembrane proteins having one or more transmembrane polypeptide domains include members of the integrin family, CD44, glycophorin, MHC Class I and II glycoproteins, EGF receptor, G protein coupled receptor (GPCR) family, porin family and other transmembrane proteins. Certain embodiments contemplate using a portion of a transmembrane polypeptide domain such as a truncated polypeptide having membrane-inserting characteristics as may be determined according to standard and well known methodologies.

Certain other embodiments relate to fusion polypeptides having a specific protein-protein association domain (e.g., Ig C_{L} polypeptide regions that mediate association to cell surface Ig H chains; β₂-microglobulin polypeptide regions that mediate association to class I MHC molecule extracellular domains, etc.), an RGD-containing polypeptide that is capable of integrin binding, a lipid raft-associating polypeptide domain, and/or a heterodimer-promoting polypeptide domain. A number of such domains are exemplified by the presently cited publications but these and related embodiments are not intended to be so limited and contemplate other specific protein-protein associating polypeptide domains that are capable of specifically associating with an extracellularly disposed region of a cell surface protein, glycoprotein, lipid, glycolipid, proteoglycan or the like, even where, importantly, such associations may in certain cases be initiated intracellularly, for instance, concomitant with the synthesis, processing, folding, assembly, transport and/or export to the cell surface of a cell surface protein. In another related embodiment, there may be included in the structure of a fusion polypeptide as described herein a domain of a protein, such as a subunit of an integrin, that is known to associate with another cell surface protein that is membrane anchored and exteriorly disposed on a cell surface. Non-limiting examples of such polypeptide domains include, for C_{L} H-chain-associating domains: (Azuma, T. and Hamaguchi, K. (1976). J Biochem 80:1023-38; Hamel et. al. (1987). J Immunol 139:3012-20; Horne et. al. (1982). J Immunol 129:660-4; Lilie et. al. (1995). J Mol Biol 248:190-201; Masuda et. al. (2006). Febs J 273:2184-94; Padlan et. al. (1986). Mol Immunol 23:951-60; Rinfret et. al. (1985). J Immunol 135:2574-81); for RGD-containing polypeptides including those that are capable of integrin binding, Heckmann, D. and Kessler, H. (2007). Methods Enzymol 426:463-503 and Takada et. al. (2007). Genome Biol 8:215; for lipid raft-associating domains, Browman et. al. 2007). Trends Cell Biol 17:394-402; Harder, T. (2004). Curr Opin Immunol 16:353-9; Hayashi, T. and Su, T. P. (2005). Life Sci 77:1612-24; Holowka, D. and Baird, B. (2001). Semin Immunol 13:99-105; Wollscheid et. al. (2004) Subcell Biochem 37:121-52).

Extracellular domains include portions of a cell surface molecule, and in particularly preferred embodiments cell surface molecules that are integral membrane proteins or that comprise a plasma membrane spanning transmembrane domain, that extend beyond the outer leaflet of the plasma membrane phospholipid bilayer when the molecule is expressed at a cell surface, preferably in a manner that exposes the extracellular domain portion of such a molecule to the external environment of the cell, also known as the extracellular milieu. Methods for determining whether a portion of a cell surface molecule comprises an extracellular domain are well known to the art and include experimental determination (e.g., direct or indirect labeling of the molecule, evaluation of whether the molecule can be structurally altered by agents to which the plasma membrane is not permeable such as proteolytic or lipolytic enzymes) or topological prediction based on the structure of the molecule (e.g., analysis of the amino acid sequence of a polypeptide) or other methodologies.

### Host Cells

According to particularly preferred embodiments a host cell is capable of utilizing recombination signals and undergoing RAG-1/RAG-2 mediated recombination and, more importantly, the recombination is controlled. Preferably the host cell is capable of cell divisions without recombination. For example, in certain embodiments one nucleic acid composition as provided herein may be introduced into a host cell, or in certain other embodiments two or more nucleic acid compositions as provided herein may be introduced into a host cell sequentially and in any order, under conditions and for a time sufficient for chromosomal integration of the nucleic acid composition(s), to obtain one, two or more chromosomally integrated nucleic acid compositions that can undergo at least two or more recombination events in the cell to form a recombined polynucleotide that encodes a polypeptide, wherein less than one of said recombination events occurs per cell cycle of the host cell. In certain embodiments, the one or more nucleic acid compositions may be maintained extrachromasomally in the host cell. As described herein, these and related embodiments permit expansion of the host cell population prior to the completion of recombination events that give rise to functionally recombined artificial immunoglobulin genes, to obtain a host cell population having protein structural diversity.

Control of recombination in such host cells may be achieved according to the compositions and methods described herein, including but not limited to the use of an operably linked recombination control element (e.g., an inducible recombination control element, which may be a tightly regulated inducible recombination control element), and/or through the use of one or more low efficiency RSSs in the nucleic acid composition(s), and/or through the use of low host cell expression levels of one or more of RAG1 or RAG-2, and/or through design of the nucleic acid composition to integrate at a chromosomal integration site offering poor accessibility to host cell recombination mechanisms (e.g., RAG1, RAG-2).

Cell lines to be used as host cells may in certain preferred embodiments additionally contain a functional TdT gene that may be expressed to provide additional diversity at the junctions (e.g., D-J and V-D junctions).

Cell lines may in certain embodiments be pre-B cells or pre-T cells that express these immunoglobulin gene rearrangement-competent cell-specific proteins (e.g., are capable of being induced to express RAG1, RAG-2 and TdT, or alternatively, constitutively express RAG1, RAG-2 and TdT but can be modified to substantially impair the expression of one, two or all three of these enzymes), or genes encoding each of these recombination-associated enzymes can be introduced into a non-B cell expression host cell, for example CHO or 293 cells. For RAG1/2 (also sometimes referred to as RAG-1 and Rag-2, see, e.g., Schatz, D G et. al. (1989) Cell 59:1035-48; Oettinger, M. A. et. al. (1990) Science 248:1517-23; for TdT see, e.g., That, T. H. & Kearney, J. F. (2004). J Immunol 173:4009-19; Koiwai, O. et. al. (1987). Biochem Biophys Res Commun 144:185-90; Peterson, R. C. et. al. (1984). Proc Natl Acad Sci USA 81:4363-7; for transfection of a host cell with all three of RAG-1, RAG-2 and TdT see, e.g., U.S. Pat. No. 5,756,323.

These and other host cells may be used according to contemplated embodiments of the present invention. For example, it has also been observed that expression of RAG-1 and/or RAG-2 is not restricted to immature developing B-cells in the bone marrow and pre-T cells of the developing thymus, but can also be observed in mature B-cells in vivo and in vitro (Maes et al., 2000 J Immunol. 165:703; Hikida et al., 1998 J Exp Med. 187:795; Casillas et. al., 1995 Mol Immunol. 32:167; Rathbun et. al., 1993 Int Immunol. 5:997, Hikida et. al., 1996 Science 274:2092). Cell lines have also been shown to continue recombination in vitro and undergo light chain replacement (Maes et. al. 2000 J Immunol. 165:703). The secondary rearrangement of Ig genes is speculated to promote receptor editing and has been shown to occur in the germinal centers of secondary lymphoid tissue like the lymph node. IL-6 has been shown to have a role in the regulation of RAG-1 and RAG-2 in mature B-cells in both inducing and terminating expression of the recombinase for secondary rearrangements. (Hillion et. al. 2007 J Immunol. 179:6790)

In addition to mature B-cells undergoing secondary rearrangement, RAG-1 and RAG-2 have also been shown to be expressed in mature T-cell lines including Jurkat T-cells. CEM cells have been shown to have V(D)J recombination activity using extrachromosomal substrates (Gauss et. al. 1998 Eur J Immunol. 28:351). Treatment of wild-type Jurkat T cells with chemical inhibitors of signaling components revealed that inhibition of Src family kinases using PP2, FK506 etc. overcame the repression of RAG-1 and resulted in increased RAG-1 expression. Mature T-cells have also been shown to reactivate recombination with treatment of anti-CD3/IL7 (Lantelme et. al. 2008 Mol Immunol. 45:328).

Recently, tumor cells of non-lymphoid origin have also been shown to express RAG-1 and RAG-2 (Zheng et. al. (2007 Mol Immunol. 44: 2221, Chen et. al. (2007 Faseb J. 21: 2931). Accordingly and without wishing to be restricted by theory, these cells may also be suitable for use as host cells in the presently described in vitro system for generating protein structural diversity. According to related embodiments that are contemplated herein, reactivation of V(D)J recombination would provide another approach to generating a suitable host cell with inducible recombinase expression. Use of other host cells is contemplated according to certain embodiments, which may vary depending on the particular mammalian genes that are employed or for other reasons, including a human cell, a non-human primate cell, a camelid cell, a hamster cell, a mouse cell, a rat cell, a rabbit cell, a canine cell, a feline cell, an equine cell, a bovine cell and an ovine cell.

Alternatively, only one of the RAG-1, or RAG-2 genes may be stably integrated into a host cell, and the other gene can be introduced by transfection to regulate whether or not recombination can take place. For example, a cell line that is stably transfected with TdT and RAG-2 would be recombinationally silent. Upon transient transfection with RAG-1, or viral infection with RAG-1, the cell lines would become recombinationally active. The skilled person will appreciate from these illustrative examples that other similar approaches may be used to control the onset of recombination in a host cell.

Another approach may be to use specific small interfering RNA (siRNA) to repress the expression in a host cell of RAG-1 and/or RAG-2 by RNA interference (RNAi) (including specific siRNAs the biosynthesis of which within a cell may be directed by introduced encoding DNA vectors having regulatory elements for controlling siRNA production), and then to relieve such repression when it is desired to induce recombination.

For instance, in certain such embodiments a cell line in which active RAG-1- and/or RAG-2-specific siRNA expression is present will be recombinationally silent. Activation of recombination occurs when RAG-1- and/or RAG-2-specific siRNA expression is shut off or repressed. Regulation of such siRNA expression may be achieved using inducible systems like the Tet system or other similar expression-regulating components. These include the Tet/on and Tet/off system (Clontech Inc., Palo Alto, Calif.), the Regulated Mammalian Expression system (Promega, Madison, Wis.), and the GeneSwitch System (Invitrogen Life Technologies, Carlsbad, Calif.). Alternatively, host cells may be transfected with an expression vector that encodes a repressing protein that prevents transcription of the inhibiting RNA.

In yet another alternative embodiment according to which RAG-1- and/or RAG-2-specific siRNA expression may regulate the recombination competence of the host cell, deletion of the introduced siRNA encoding sequences by use of the Cre/Lox recombinase system (e.g., Sauer, 1998 Methods 14:381; Kaczmarczyk et al., 2001 Nucleic Acids Res 29:E56; Sauer, 2002 Endocrine 19:221; Kondo et al., 2003 Nucleic Acids Res 31:e76) may also permit activation of recombination mechanisms. Activation of recombination capability in a host cell may also be achieved by transfecting or infecting an expression construct containing the repressed gene with modified codons so that it is not inhibited by the siRNA molecules.

Substantial impairment of the expression of one or more recombination control elements (e.g., a RAG-1 gene, or RAG-2 gene) may be achieved by any of a variety of methods that are well known in the art for blocking specific gene expression, including antisense inhibition of gene expression, ribozyme mediated inhibition of gene expression, siRNA mediated inhibition of gene expression, cre recombinase regulation of expression control elements using the Cre/Lox system in the design of constructs encoding one or more recombination control elements, or other molecular regulatory strategies. As used herein, expression of a gene encoding a recombination control element is substantially impaired by any such method for inhibiting when host cells are substantially but not necessarily completely depleted of functional DNA or functional mRNA encoding the recombination control element, or of the relevant RAG-1, or RAG-2 polypeptide. Recombination control element expression is substantially impaired when cells are preferably at least 50% depleted of DNA or mRNA encoding the endogenous RAG-1, and/or RAG-2 polypeptide (as detected using high stringency hybridization) or 50% depleted of detectable RAG-1 and/or RAG-2 polypeptide (e.g., as measured by Western immunoblot); and more preferably at least 75% depleted of detectable RAG-1, and/or RAG-2 polypeptide. Most preferably, recombination control element expression is substantially impaired when host cells are depleted of >90% of their endogenous RAG-1 and/or RAG-2 DNA, mRNA, or polypeptide.

It will be appreciated that certain embodiments disclosed herein relate to the use of nucleic vectors for the assembly of the nucleic acid compositions for generating protein structural diversity, and also for RAG-1, RAG-2 and/or TdT gene expression and for regulatory constructs such as siRNA regulators of RAG-1, RAG-2 and/or TdT expression. A wide variety of suitable nucleic acid vectors are known in the art and may be employed as described or according to conventional procedures, including modifications, as described for example in Sambrook et al., Molecular Cloning: A Laboratory Manual, Cold Spring Harbor Laboratories, Cold Spring Harbor, N.Y., 1989; Ausubel et al., Current Protocols in Molecular Biology, Greene Publ. Assoc. Inc. & John Wiley & Sons, Inc., Boston, Mass., 1993); Maniatis et al. (Molecular Cloning, Cold Spring Harbor Laboratory, Plainview, N.Y., 1982) and elsewhere.

Other vectors that may be adapted for use according to certain herein disclosed embodiments include those described by Choi, S. & Kim, U. J. (2001) 175:57-68; Fabb, S. A. & Ragoussis, J. (1995) Mol Cell Biol Hum Dis Ser 5:104-24; Monaco, Z. L. & Moralli, D. (2006). Biochem Soc Trans 34:324-7; Ripoll et. al. (1998). Gene 210:163-72. Also contemplated are the use of protoplast fusion systems such as those described by Caporale et. al. (1990). Gene 87:285-9; Ferguson et. al. (1986). J Biol Chem 261:14760-3, Sandri-Goldin et. al. (1981). Mol Cell Biol 1:743-52; and yeast artificial chromosome (YAC) spheroblast fusion as described by Davies, N. P. and Huxley, C. (1996). Methods Mol Biol 54:281-92; Gnirke et. al (1991). Embo J 10:1629-34; Ikeno et. al. (1998). Nat Biotechnol 16:431-9; Jakobovits, A et. al. (1993). Nature 362:255-8; Pavan et. al. (1990). Mol Cell Biol 10:4163-9. In certain embodiments the nucleic acid compositions for generating protein structural diversity as provided herein are stably integrated into host cell chromosomes using known methodologies and where such integration can be confirmed according to established techniques (e.g., Sambrook et al., 1989; Ausubel et al., 1993; Maniatis et al. 1982). Related embodiments contemplate chromosomal EBV elements that mediate integration, and other embodiments contemplate extrachromosomal maintenance of natural or artificial centromere-containing constructs.

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); and elsewhere.

The DNA sequence in the vector (e.g., an expression vector) is operatively linked to at least one appropriate expression control sequences (e.g., a promoter or a regulated promoter) to direct mRNA synthesis. Representative examples of such expression control sequences include LTR or SV40 promoter, the E. coli lac or trp, the phage lambda P_{L} promoter and other promoters known to control expression of genes in prokaryotic or eukaryotic cells or their viruses. Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P_{R}, P_{L} and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding an immunoglobulin region or region of a non-Ig protein.

In certain preferred embodiments the expression control sequence is a “regulated promoter”, which may be a promoter as provided herein and may also be a repressor binding site, an activator binding site or any other regulatory sequence that controls expression of a nucleic acid sequence as provided herein. In certain particularly preferred embodiments the regulated promoter is a tightly regulated promoter that is specifically inducible and that permits little or no transcription of nucleic acid sequences under its control in the absence of an induction signal, as is known to those familiar with the art and described, for example, in Guzman et al. (1995 J. Bacteriol. 177:4121), Carra et al. (1993 EMBO J. 12:35), Mayer (1995 Gene 163:41), Haldimann et al. (1998 J. Bacteriol. 180:1277), Lutz et al. (1997 Nuc. Ac. Res. 25:1203), Allgood et al. (1997 Curr. Opin. Biotechnol. 8:474) and Makrides (1996 Microbiol. Rev. 60:512), all of which are hereby incorporated by reference. In other preferred embodiments of the invention a regulated promoter is present that is inducible but that may not be tightly regulated. In certain other preferred embodiments a promoter is present in the recombinant expression construct of the invention that is not a regulated promoter; such a promoter may include, for example, a constitutive promoter such as an insect polyhedrin promoter. The expression construct also contains a ribosome binding site for translation initiation and a transcription terminator. The vector may also include appropriate sequences for amplifying expression.

Transcription of the DNA encoding the polypeptides of the present invention by higher eukaryotes may be increased by inserting an enhancer sequence into the vector. Enhancers are cis-acting elements of DNA, usually about from 10 to 300 bp that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers.

As noted above, in certain embodiments the vector may be a viral vector such as a retroviral vector. For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus.

The viral vector includes one or more promoters. Suitable promoters which may be employed include, but are not limited to, the retroviral LTR; the SV40 promoter; and the human cytomegalovirus (CMV) promoter described in Miller, et al., Biotechniques 7:980-990 (1989), or any other promoter (e.g., cellular promoters such as eukaryotic cellular promoters including, but not limited to, the histone, pol III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein, and may be from among either regulated promoters or promoters as described above.

The retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides or fusion proteins. Such retroviral vector particles then may be employed, to transduce eukaryotic cells, either in vitro or in vivo. The transduced eukaryotic cells will express the nucleic acid sequence(s) encoding the polypeptide or fusion protein. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Also contemplated in certain embodiments are replicating and non-replicating episomal vectors for transient expression. Replicating vectors contain origin sequences that promote plasmid replication in the presence of the appropriate trans factors. The SV40 and polyoma origins and respective T-antigens are non-limiting examples. Also contemplated are stably maintained episomal expression vectors. Episomal plasmids are usually based on sequences from DNA viruses, such as BK virus, bovine papilloma virus 1 and Epstein-Barr virus (see, for example, Van Craenenbroeck, K., et al., 2000, Eur. J. Biochem. 267:5665-5678). These vectors contain a viral origin of DNA replication and a viral early gene(s), the product of which activates the viral origin and thus allows the episome to reside in the transfected host cell line in a well-controlled manner. Episomal vectors are plasmid constructions that replicate in both eukaryotic and prokaryotic cells and can therefore also be “shuttled” from one host cell system to another.

As described herein, certain embodiments relate to compositions that are capable of delivering the described nucleic acid molecules. Such compositions include recombinant viral vectors (e.g., retroviruses (see WO 90/07936, WO 91/02805, WO 93/25234, WO 93/25698, and WO 94/03622), adenovirus (see Berkner, Biotechniques 6:616-627, 1988; Li et al., Hum. Gene Ther. 4:403-409, 1993; Vincent et al., Nat. Genet. 5:130-134, 1993; and Kolls et al., Proc. Natl. Acad. Sci. USA 91:215-219, 1994), pox virus (see U.S. Pat. No. 4,769,330; U.S. Pat. No. 5,017,487; and WO 89/01973)), recombinant expression construct nucleic acid molecules complexed to a polycationic molecule (see WO 93/03709), and nucleic acids associated with liposomes (see Wang et al., Proc. Natl. Acad. Sci. USA 84:7851, 1987). In certain embodiments, the DNA may be linked to killed or inactivated adenovirus (see Curiel et al., Hum. Gene Ther. 3:147-154, 1992; Cotton et al., Proc. Natl. Acad. Sci. USA 89:6094, 1992). Other suitable compositions include DNA-ligand (see Wu et al., J. Biol. Chem. 264:16985-16987, 1989) and lipid-DNA combinations (see Feigner et al., Proc. Natl. Acad. Sci. USA 84:7413-7417, 1989).

Various mammalian cell culture systems can also be employed to express recombinant protein. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, calcium phosphate transfection, DEAE-Dextran mediated transfection, or electroporation (Davis et al., 1986 Basic Methods in Molecular Biology). Additional methods include spheroplast fusion and protoplast fusion.

### Nucleic Acids

The nucleic acids of the present invention, also referred to herein as polynucleotides, may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. A coding sequence which encodes an immunoglobulin or a region thereof (e.g., a V region, a D segment, a J region, a C region, etc.), a non-Ig protein or region thereof, or a fusion polypeptide for use according to the present embodiments may be identical to the coding sequence known in the art for any given gene regions or fusion polypeptide domains (e.g., membrane anchor domains, extracellular domain-associating polypeptides, etc.), or may be a different coding sequence, which, as a result of the redundancy or degeneracy of the genetic code, encodes the same immunoglobulin region, non-Ig protein region or fusion polypeptide.

The nucleic acids for use according to the embodiments described herein may include, but are not limited to: only the coding sequence for an immunoglobulin, non-immunoglobulin protein or fusion polypeptide; the coding sequence for the immunoglobulin, non-immunoglobulin protein or fusion polypeptide and additional coding sequence; the coding sequence for the immunoglobulin, non-immunoglobulin or fusion polypeptide (and optionally additional coding sequence) and non-coding sequence, such as introns or non-coding sequences 5′ and/or 3′ of the coding sequence, which for example may further include but need not be limited to one or more regulatory nucleic acid sequences that may be a regulated or regulatable promoter, enhancer, other transcription regulatory sequence, repressor binding sequence, translation regulatory sequence or any other regulatory nucleic acid sequence. Thus, the term “nucleic acid encoding” or “polynucleotide encoding” an immunoglobulin, non-immunoglobulin protein or fusion protein encompasses a nucleic acid which includes only coding sequence, as well as a nucleic acid which includes additional coding and/or non-coding sequence(s).

Nucleic acids and oligonucleotides for use as described herein can be synthesized by any method known to those of skill in this art (see, e.g., WO 93/01286, U.S. application Ser. No. 07/723,454; U.S. Pat. No. 5,218,088; U.S. Pat. No. 5,175,269; U.S. Pat. No. 5,109,124). Identification of oligonucleotides and nucleic acid sequences for use in the present invention involves methods well known in the art. For example, the desirable properties, lengths and other characteristics of useful oligonucleotides are well known. In certain embodiments, synthetic oligonucleotides and nucleic acid sequences may be designed that resist degradation by endogenous host cell nucleolytic enzymes by containing such linkages as: phosphorothioate, methylphosphonate, sulfone, sulfate, ketyl, phosphorodithioate, phosphoramidate, phosphate esters, and other such linkages that have proven useful in antisense applications (see, e.g., Agrwal et al., Tetrehedron Lett. 28:3539-3542 (1987); Miller et al., J. Am. Chem. Soc. 93:6657-6665 (1971); Stec et al., Tetrehedron Lett. 26:2191-2194 (1985); Moody et al., Nucl. Acids Res. 12:4769-4782 (1989); Uznanski et al., Nucl. Acids Res. (1989); Letsinger et al., Tetrahedron 40:137-143 (1984); Eckstein, Annu. Rev. Biochem. 54:367-402 (1985); Eckstein, Trends Biol. Sci. 14:97-100 (1989); Stein In: Oligodeoxynucleotides. Antisense Inhibitors of Gene Expression, Cohen, Ed, Macmillan Press, London, pp. 97-117 (1989); Jager et al., Biochemistry 27:7237-7246 (1988)).

As known in the art “similarity” between two polypeptides is determined by comparing the amino acid sequence and conserved amino acid substitutes thereto of the polypeptide to the sequence of a second polypeptide. Fragments or portions of the nucleic acids encoding polypeptides of the present invention may be used to synthesize full-length nucleic acids of the present invention. As used herein, “% identity” refers to the percentage of identical amino acids situated at corresponding amino acid residue positions when two or more polypeptide are aligned and their sequences analyzed using a gapped BLAST algorithm (e.g., Altschul et al., 1997 Nucl. Ac. Res. 25:3389) which weights sequence gaps and sequence mismatches according to the default weightings provided by the National Institutes of Health/NCBI database (Bethesda, Md.; see www.ncbi.nlm.nih.gov/cgi-bin/BLAST/nph-newblast).

Determination of the three-dimensional structures of representative polypeptides (e.g., immunoglobulins, non-Ig proteins, membrane anchor domain polypeptides, specific protein-protein association domains, etc.) may be made through routine methodologies such that substitution of one or more amino acids with selected natural or non-natural amino acids can be virtually modeled for purposes of determining whether a so derived structural variant retains the space-filling properties of presently disclosed species. See, for instance, Donate et al., 1994 Prot. Sci. 3:2378; Bradley et al., Science 309: 1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007). Some additional non-limiting examples of computer algorithms that may be used for these and related embodiments, such as for rational design of membrane anchor domains or specific protein-protein association domains as provided herein, include Desktop Molecular Modeler (See, for example, Agboh et al., J. Biol. Chem., 279, 40: 41650-57 (2004)), which allows for determining atomic dimensions from spacefilling models (van der Waals radii) of energy-minimized conformations; GRID, which seeks to determine regions of high affinity for different chemical groups, thereby enhancing binding, Monte Carlo searches, which calculate mathematical alignment, and CHARMM (Brooks et al. (1983) J. Comput. Chem. 4:187-217) and AMBER (Weiner et al (1981) J. Comput. Chem. 106: 765), which assess force field calculations, and analysis (see also, Eisenfield et al. (1991) Am. J. Physiol. 261:C376-386; Lybrand (1991) J. Pharm. Belg. 46:49-54; Froimowitz (1990) Biotechniques 8:640-644; Burbam et al. (1990) Proteins 7:99-111; Pedersen (1985) Environ. Health Perspect. 61:185-190; and Kini et al. (1991) J. Biomol. Struct. Dyn. 9:475-488).

A truncated molecule may be any molecule that comprises less than a full length version of the molecule. Truncated molecules provided by the present invention may include truncated biological polymers, and in preferred embodiments of the invention such truncated molecules may be truncated nucleic acid molecules or truncated polypeptides. Truncated nucleic acid molecules have less than the full length nucleotide sequence of a known or described nucleic acid molecule, where such a known or described nucleic acid molecule may be a naturally occurring, a synthetic or a recombinant nucleic acid molecule, so long as one skilled in the art would regard it as a full length molecule. Thus, for example, truncated nucleic acid molecules that correspond to a gene sequence contain less than the full length gene where the gene comprises coding and non-coding sequences, promoters, enhancers and other regulatory sequences, flanking sequences and the like, and other functional and non-functional sequences that are recognized as part of the gene. In another example, truncated nucleic acid molecules that correspond to a mRNA sequence contain less than the full length mRNA transcript, which may include various translated and non-translated regions as well as other functional and non-functional sequences.

In other preferred embodiments, truncated molecules are polypeptides that comprise less than the full length amino acid sequence of a particular protein or polypeptide component. As used herein “deletion” has its common meaning as understood by those familiar with the art, and may refer to molecules that lack one or more of a portion of a sequence from either terminus or from a non-terminal region, relative to a corresponding full length molecule, for example, as in the case of truncated molecules provided herein. Truncated molecules that are linear biological polymers such as nucleic acid molecules or polypeptides may have one or more of a deletion from either terminus of the molecule or a deletion from a non-terminal region of the molecule, where such deletions may be deletions of 1-1500 contiguous nucleotide or amino acid residues, preferably 1-500 contiguous nucleotide or amino acid residues and more preferably 1-300 contiguous nucleotide or amino acid residues, including deletions of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41-50, 51-74, 75-100, 101-150, 151-200, 201-250 or 251-299 contiguous nucleotide or amino acid residues. In certain particularly preferred embodiments truncated nucleic acid molecules may have a deletion of 270-330 contiguous nucleotides. In certain other particularly preferred embodiments truncated polypeptide molecules may have a deletion of 80-140 contiguous amino acids.

The present invention further relates to variants of the herein referenced nucleic acids which encode fragments, analogs and/or derivatives of an immunoglobulin, non-immunoglobulin protein or fusion polypeptide. The variants of the nucleic acids encoding such polypeptides may be naturally occurring allelic variants of the nucleic acids or non-naturally occurring variants. As is known in the art, an allelic variant is an alternate form of a nucleic acid sequence which may have at least one of a substitution, a deletion or an addition of one or more nucleotides, any of which does not substantially alter the function of the encoded polypeptide.

Variants and derivatives of immunoglobulin, non-immunoglobulin protein or fusion polypeptide may be obtained by mutations of nucleotide sequences encoding such polypeptides or any portion thereof. Alterations of the native amino acid sequence may be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making such alterations are disclosed by Walder et al. (Gene 42:133, 1986); Bauer et al. (Gene 37:73, 1985); Craik (BioTechniques, January 1985, 12-19); Smith et al. (Genetic Engineering: Principles and Methods BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462.

As an example, modification of DNA may be performed by site-directed mutagenesis of DNA encoding the protein combined with the use of DNA amplification methods using primers to introduce and amplify alterations in the DNA template, such as PCR splicing by overlap extension (SOE). Site-directed mutagenesis is typically effected using a phage vector that has single- and double-stranded forms, such as M13 phage vectors, which are well-known and commercially available. Other suitable vectors that contain a single-stranded phage origin of replication may be used (see, e.g., Veira et al., Meth. Enzymol. 15:3, 1987). In general, site-directed mutagenesis is performed by preparing a single-stranded vector that encodes the protein of interest. An oligonucleotide primer that contains the desired mutation within a region of homology to the DNA in the single-stranded vector is annealed to the vector followed by addition of a DNA polymerase, such as E. coli DNA polymerase I (Klenow fragment), which uses the double stranded region as a primer to produce a heteroduplex in which one strand encodes the altered sequence and the other the original sequence. The heteroduplex is introduced into appropriate bacterial cells and clones that include the desired mutation are selected. The resulting altered DNA molecules may be expressed recombinantly in appropriate host cells to produce the modified protein.

Equivalent DNA constructs that encode various additions or substitutions of amino acid residues or sequences, or deletions of terminal or internal residues or sequences not needed for biological activity are also encompassed by the invention. For example, sequences encoding Cys residues that are not desirable or essential for biological activity can be altered to cause the Cys residues to be deleted or replaced with other amino acids, preventing formation of incorrect or undesirable intramolecular disulfide bridges upon renaturation.

### Immunoglobulins

As described herein and as also known in the art, immunoglobulins comprise products of a gene family the members of which exhibit a high degree of sequence conservation, such that amino acid sequences of two or more immunoglobulins or immunoglobulin domains or regions or portions thereof (e.g., VH domains, VL domains, hinge regions, CH2 constant regions, CH3 constant regions) can be aligned and analyzed to identify portions of such sequences that correspond to one another, for instance, by exhibiting pronounced sequence homology. (See, e.g., Kabat et al., Sequences of Proteins of Immunological Interest, Edition: 5, 1992 DIANE Publishing, 1992, Darby, P A; Tomlinson et al., 1992 J Mol Biol 227:776; Milner et al., 1995 Ann N Y Acad Sci 764:50.) Determination of sequence homology may be readily determined with any of a number of sequence alignment and analysis tools, including computer algorithms well known to those of ordinary skill in the art, such as Align or the BLAST algorithm (Altschul, J. Mol. Biol. 219:555-565, 1991; Henikoff and Henikoff, Proc. Natl. Acad. Sci. USA 89:10915-10919, 1992), which is available at the NCBI website (http://www/ncbi.nlm.nih.gov/cgi-bin/BLAST). Default parameters may be used.

Portions of a particular immunoglobulin reference sequence and of any one or more additional immunoglobulin sequences of interest that may be compared to the reference sequence are regarded as “corresponding” sequences, regions, fragments or the like, based on the convention for numbering immunoglobulin amino acid positions according to Kabat, Sequences of Proteins of Immunological Interest, (5^{th} ed. Bethesda, Md.: Public Health Service, National Institutes of Health (1991)). For example, according to this convention, the immunoglobulin family to which an immunoglobulin sequence of interest belongs is determined based on conservation of variable region polypeptide sequence invariant amino acid residues, to identify a particular numbering system for the immunoglobulin family, and the sequence(s) of interest can then be aligned to assign sequence position numbers to the individual amino acids which comprise such sequence(s). Preferably at least 70%, more preferably at least 80%-85% or 86%-89%, and still more preferably at least 90%, 92%, 94%, 96%, 98% or 99% of the amino acids in a given amino acid sequence of at least 1000, more preferably 700-950, more preferably 350-700, still more preferably 100-350, still more preferably 80-100, 70-80, 60-70, 50-60, 40-50 or 30-40 consecutive amino acids of a sequence, are identical to the amino acids located at corresponding positions in a reference sequence such as those disclosed by Kabat et al. (1991) or Kabat et al. (1992) or in a similar compendium of related immunoglobulin sequences, such as may be generated from public databases (e.g., Genbank, SwissProt, etc.) using sequence alignment tools as described above. In certain preferred embodiments, an immunoglobulin sequence of interest or a region, portion, derivative or fragment thereof is greater than 95% identical to a corresponding reference sequence, and in certain preferred embodiments such a sequence of interest may differ from a corresponding reference at no more than 1, 2, 3, 4, 5, 6, 7, 8, 9 or 10 amino acid positions.

Human immunoglobulin gene libraries are currently generated by any number of techniques with which those having ordinary skill in the art will be familiar. Such methods include but are not limited to, Epstein Barr Virus (EBV) transformation of human peripheral blood cells (e.g., containing B lymphocytes), in vitro immunization of human B cells, fusion of spleen cells from immunized transgenic mice carrying human immunoglobulin genes inserted by yeast artificial chromosomes (YAC), isolation from human immunoglobulin V region phage libraries, or other procedures as known in the art and based on the disclosure herein. See, e.g., U.S. Pat. No. 5,877,397; Bruggemann et al., 1997 Curr. Opin. Biotechnol. 8:455-58; Jakobovits et al., 1995 Ann. N.Y. Acad. Sci. 764:525-35. In the described human immunoglobulin gene-carrying transgenic mice, human immunoglobulin heavy and light chain genes have been artificially introduced by genetic engineering in germline configuration, and the endogenous murine immunoglobulin genes have been inactivated. See, e.g., Bruggemann et al., 1997 Curr. Opin. Biotechnol. 8:455-58. For example, human immunoglobulin transgenes may be mini-gene constructs, or transloci on yeast artificial chromosomes, which undergo B cell-specific DNA rearrangement and hypermutation in the mouse lymphoid tissue. See, Bruggemann et al., 1997 Curr. Opin. Biotechnol. 8:455-58.

According to certain embodiments, structurally diverse non-human, human, or humanized immunoglobulin heavy chain and/or light chain variable regions such as can be generated using the compositions and methods disclosed herein, may be constructed as single chain Fv (sFv) polypeptide fragments (single chain antibodies). See, e.g., Bird et al., 1988 Science 242:423-426; Huston et al., 1988 Proc. Natl. Acad. Sci. USA 85:5879-5883. Multi-functional sFv fusion proteins may be generated by linking a polynucleotide sequence encoding an sFv polypeptide in-frame with at least one polynucleotide sequence encoding any of a variety of known effector proteins. These methods are known in the art, and are disclosed, for example, in EP-B1-0318554, U.S. Pat. No. 5,132,405, U.S. Pat. No. 5,091,513, and U.S. Pat. No. 5,476,786. By way of example, effector proteins may include immunoglobulin constant region sequences. See, e.g., Hollenbaugh et al., 1995 J. Immunol. Methods 188:1-7. Other examples of effector proteins are enzymes. As a non-limiting example, such an enzyme may provide a biological activity for therapeutic purposes (see, e.g., Siemers et al., 1997 Bioconjug Chem. 8:510-19), or may provide a detectable activity, such as horseradish peroxidase-catalyzed conversion of any of a number of well-known substrates into a detectable product, for diagnostic uses. Still other examples of sFv fusion proteins include Ig-toxin fusions, or immunotoxins, wherein the sFv polypeptide is linked to a toxin. Those having ordinary skill in the art will appreciate that a wide variety of polypeptide sequences have been identified that, under appropriate conditions, are toxic to cells. As used herein, a toxin polypeptide for inclusion in an immunoglobulin-toxin fusion protein may be any polypeptide capable of being introduced to a cell in a manner that compromises cell survival, for example, by directly interfering with a vital function or by inducing apoptosis. Toxins thus may include, for example, ribosome-inactivating proteins, such as Pseudomonas aeruginosa exotoxin A, plant gelonin, bryodin from Bryonia dioica, or the like. See, e.g., Thrush et al., 1996 Annu. Rev. Immunol. 14:49-71; Frankel et al., 1996 Cancer Res. 56:926-32. Numerous other toxins, including chemotherapeutic agents, antimitotic agents, antibiotics, inducers of apoptosis (or “apoptogens”, see, e.g., Green and Reed, 1998, Science 281:1309-1312), or the like, are known to those familiar with the art, and the examples provided herein are intended to be illustrative without limiting the scope and spirit of the invention.

A sFv may be fused to peptide or polypeptide domains that permit detection of specific binding between the fusion protein and a desired antigen. For example, the fusion polypeptide domain may be an affinity tag polypeptide. Binding of the sFv fusion protein to a binding partner (e.g., an antigen of interest such as a diagnostic or therapeutic target molecule) may therefore be detected using an affinity polypeptide or peptide tag, such as an avidin, streptavidin or a His (e.g., polyhistidine) tag, by any of a variety of techniques with which those skilled in the art will be familiar. Detection techniques may also include, for example, binding of an avidin or streptavidin fusion protein to biotin or to a biotin mimetic sequence (see, e.g., Luo et al., 1998 J. Biotechnol. 65:225 and references cited therein), direct covalent modification of a fusion protein with a detectable moiety (e.g., a labeling moiety), noncovalent binding of the fusion protein to a specific labeled reporter molecule, enzymatic modification of a detectable substrate by a fusion protein that includes a portion having enzyme activity, or immobilization (covalent or non-covalent) of the fusion protein on a solid-phase support.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Specific Constructs for the Recombination Control Elements and Mediators of Junctional Diversity

This Example describes the sequences of the recombination control elements and mediators of junctional diversity [SEQ ID NOS:1-6]. These elements were codon optimized (Geneart, Inc., Burlingame, Calif.) for translation in mammalian cells and contain 5′ HindIII and 3′ XbaI restriction sites to facilitate cloning into expression vectors containing CMV or SV40 promoters. The RAG-1 polynucleotide [SEQ ID NO:1] encodes human RAG-1 polypeptide [SEQ ID NO:2], and was gene optimized for expression in mammalian cells. The translation product of this construct was identical to the deduced translation of RAG-1 mRNA in the Genbank database (NM_{—}000448). The polynucleotide sequence is provided in SEQ ID NO:1 and the amino acid sequence is provided in SEQ ID NO:2. The RAG-2 polynucleotide [SEQ ID NO:3] encodes the human RAG-2 polypeptide [SEQ ID NO:4], and was codon optimized (Geneart, Inc., Toronto, Canada) for expression in mammalian cells. The translation product of this construct was identical to the deduced translation of RAG-2 mRNA in the Genbank database (NM_{—}000536). The polynucleotide sequence is provided in SEQ ID NO:3 and the amino acid sequence is provided in SEQ ID NO:4. ITS-5 [SEQ ID NO:5] encoded human TdT, codon optimized (Geneart, Inc., Burlingame, Calif.) for expression in mammalian cells. The translation product of ITS-5 was identical to the deduced translation of TdT mRNA in the Genbank sequence (NM_{—}004088). The polynucleotide sequence is provided in SEQ ID NO:5 and the amino acid sequence is provided in SEQ ID NO:6. RAG-1 and RAG-2 were cloned into pcDNA3.1 and were shown to mediate VDJ recombination (described below).

### Example 2

### RAG-1/RAG-2 Mediated Recombination

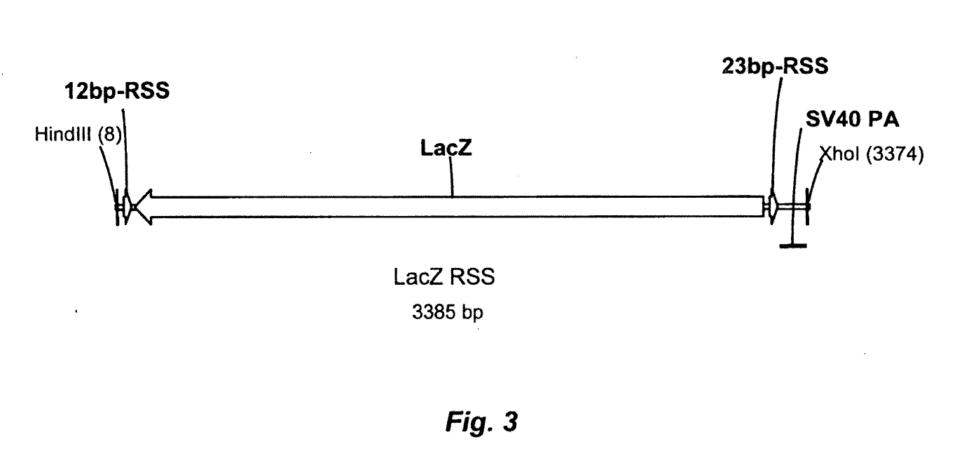

RAG-1/RAG-2 mediated recombination was targeted through cis recombination signal sequences (RSS). DNA containing the E. coli LacZ gene flanked by RSS sequenes was custom synthesized by Geneart Inc. (Toronto, Canada) with HindIII and XhoI ends for subsequent cloning (LacZ-RSS, SEQ ID NO:7). A recombination substrate vector, V25, was generated by cloning the HindIII/XhoI restriction fragment containing coding sequence for the beta-galactosidase reporter flanked by upstream and downstream RSSs, LacZ-RSS, into plasmid vector pcDNA3.1(+) (Invitrogen, Carlsbad, Calif.). FIG. 3 shows a schematic diagram of LacZ-RSS. The polynucleotide sequence of LacZ-RSS is provided in SEQ ID NO:7 and the translated amino acid sequence is provided in SEQ ID NO:8. The recombination substrate encoded the bacterial enzyme LacZ (beta-galactosidase) and was codon optimized for expression in mammalian cells, such that the LacZ was flanked by two recombination signal sequences in the same orientation. The sequences of the RSSs were as follows:

12-bp RSS:
[SEQ ID NO: 18]
CACAGTGCTCCAGGGCTGAACAAAAACC
23-bp RSS:
[SEQ ID NO: 19]
CACAGTGGTAGTACTCCACTGTCTGGGTGTACAAAAACC

The LacZ coding sequence was initially in the reverse orientation relative to the CMV promoter and thus no beta-galactosidase was expressed when the vector was tranfected into cells. An SV40 polyadenylation signal next to the 23-bp RSS ensured that unintended expression of lacZ was minimal prior to recombination. In the presence of RAG-1/RAG-2, the orientation of the LacZ coding sequence was reversed since the recombination signals were in the same orientation, generating an inversional event. Following recombination LacZ coding sequence was placed in the same orientation as the CMV promoter and beta-galactosidase was expressed. Beta-galactosidase enzymatic activity expressed by cells that had undergone RAG-1/RAG-2 mediated recombination was assayed with colorimetric β-gal substrates, by enzyme linked immunosorbent assay (ELISA) and by microscopy.

The RAG-1 and RAG-2 constructs were confirmed to mediate recombination using the following procedure. 293-H cells were transfected according to the supplier's recommendations (Invitrogen, Carlsbad, Calif., Cat. No. 11631-017). Cells were seeded at 20,000 cells/well in a tissue culture treated 96-well plate and incubated overnight. The next day, cells were transfected with Lipofectamine 2000 (Invitrogen, Carlsbad, Calif., Cat. No. 11668-019) according to the manufacturer's recommendations. Cells were transfected with 67 ng of the LacZ-RSS plasmid, 0 or 33 ng of the RAG-2 plasmid and 0, 8, 17, 33 or 67 ng of the RAG-1 plasmid. Carrier plasmid was added such that all samples received the same total amount of DNA. Two days after transfection, cell lysates were prepared and beta-galactosidase activity was determined using the colorimetric substrate chlorophenol red-β-D-galactopyranoside (Sigma, St. Louis, Mo., Cat. No. 59767-25MG-F).

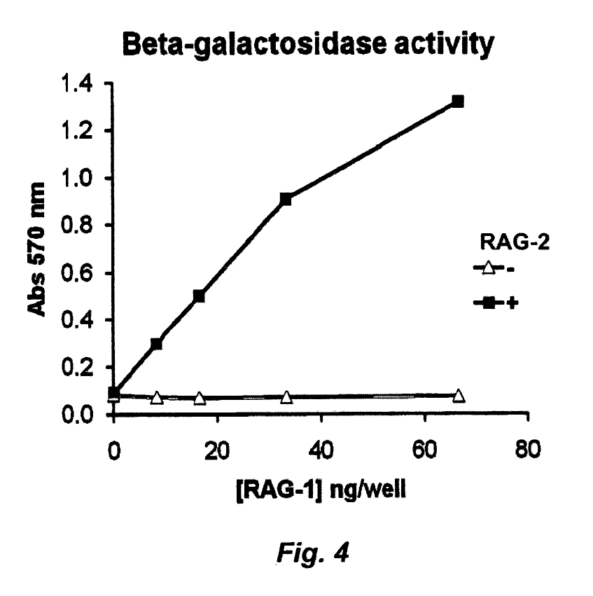

The results shown in FIG. 4 demonstrated that recombination was dependent on the expression of both RAG-1 and RAG-2. The figure also shows that recombination activity increased with increasing amounts of the RAG-1 plasmid during the transfection step.

### Example 3

### RAG-1/RAG-2 Induced Recombination of an Integrated Substrate

A stable cell line integrated with the recombination substrate V25, prepared as described above (e.g., Example 2), was generated by transfection of HEK-293 cells with Lipofectamine™ 2000 according to the manufacturer's instructions (Invitrogen, Carlsbad, Calif.). Stable pools of transfected cells were selected using 1 mg/ml G418. Stably selected cell pools were subsequently split into a 96 well plate and 24 hours later wells were transiently transfected with equal amounts of the RAG1 and RAG2 expression vectors (RAG-1 and RAG-2 coding sequences, respectively, cloned into pcDNA3.1(+) (Invitrogen, Carlsbad, Calif.). Forty-eight hours following transfection cells were fixed and stained for beta-galatosidase activity according to the manufacturer's instructions (Cat. #K1465-01, Invitrogen, Carlsbad, Calif.), by which a detectable blue stain indicates beta-galactosidase activity.

Staining was allowed to proceed overnight. There were no blue cells observed amongst 293 cells that were stably integrated with V25 but that had not been transiently transfected with RAG-1 and RAG-2. Amongst 293 cells that were stably integrated with V25 and transiently transfected with RAG-1 and RAG-2, blue stained cells were readily detectable by light microscopy, with multiple blue stained cells observed per field. The results demonstrated that recombination of the integrated substrate was successfully induced by the transient expression of RAG-1 and RAG-2.

### Example 4

### Diversifying an Immunoglobulin Heavy Chain

An antibody (immunoglobulin) molecule is a heterodimer comprised of two subunits, a heavy chain and a light chain. This example demonstrates the assembly of intact antibodies as the result of the recombination of surface Ig heavy chain encoding VDJ recombination substrates in HEK-293 cells transiently expressing RAG-1 and RAG-2 and the human kappa light chain.

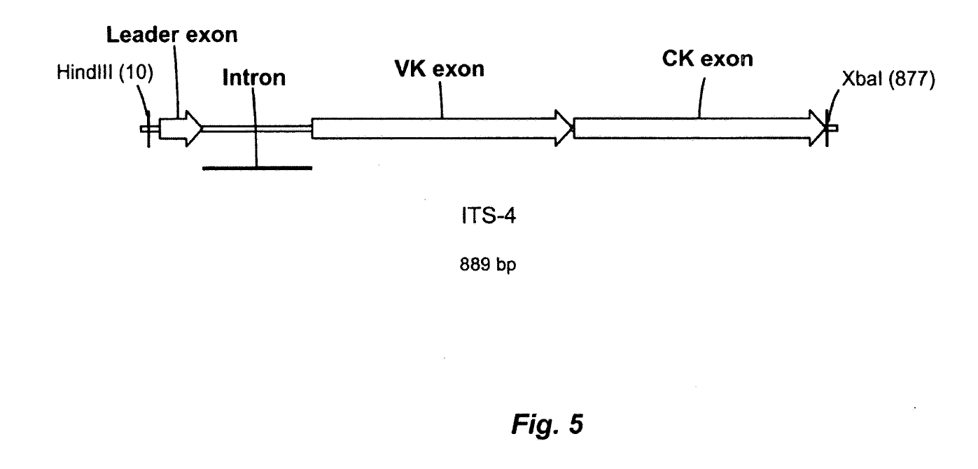

A light chain vector encoding a functional immunoglobulin kappa chain was prepared containing a leader exon, an intron, a V kappa exon and a constant kappa exon, and was designated ITS-4. The sequence of the constant region was based on the Genebank sequence NG_{—}000834. The entire coding sequence was codon optimized (Geneart, Inc., Burlingame, Calif.) for expression in mammalian cells. FIG. 5 shows a schematic diagram of ITS-4. The polynucleotide sequence is provided in SEQ ID NO:9 and the amino acid sequence is provided in SEQ ID NO:10.

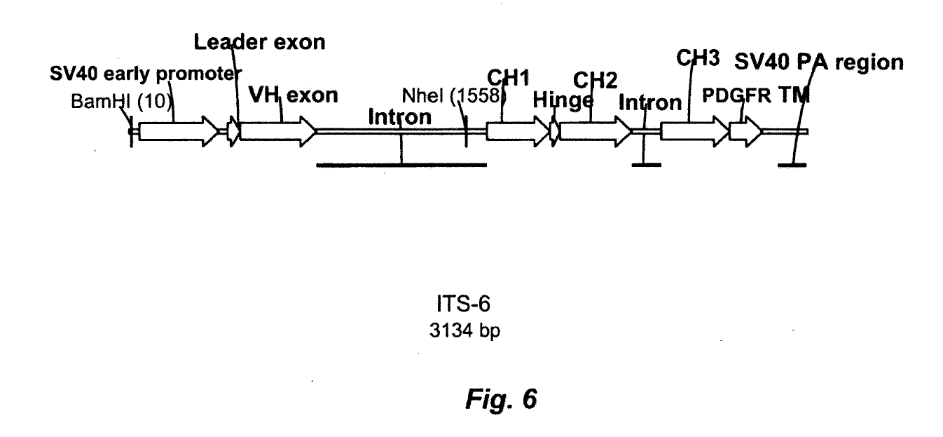

A heavy chain vector designed to express IgG on the surface of the cell was also generated, and designated ITS-6. ITS-6 [SEQ ID NO:11] encoded a functional human IgG1 antibody heavy chain [SEQ ID NO:12] that localized to the cell surface and was anchored to the plasma membrane by a transmembrane domain derived from the human platelet derived growth factor receptor (PDGFR). A schematic diagram of ITS-6 is shown in FIG. 6. Expression was driven by a SV40 promoter. An SV40 polyadenylation signal was present at the downstream (3′) end of the construct. There were two introns in the construct, one between the VDJH exon (preassembled heavy chain exon) and the CH1 exon, and the other between the CH2 exon and the CH3 exon. The restriction enzyme sites BamHI and NheI facilitated substitution of the variable domain for VDJ substrates. Transfection of HEK-293 cells with both ITS-6 and ITS-4 (co-transfection) resulted in human IgG expressed on the surface of cells. The ITS-6 vector was the backbone for all additional tripartite antibody diversification vectors. The polynucleotide sequence of ITS-6 is provided in SEQ ID NO:11 and the amino acid sequence is provided in SEQ ID NO:12.

The vector ITS-6 [SEQ ID NO:6] was modified to remove the functional antibody encoding sequences and replace them with VH gene segments with appropriate recombination signal sequences (RSSs), D gene segments with and appropriate RSSs, and J gene segments with appropriate RSSs, to create recombination vectors designated V64 [SEQ ID NOS:14-15], V67 [SEQ ID NO:16] and V86 [SEQ ID NO:17]. In each vector, each V segment had an upstream SV40 early promoter and a downstream 23-bp RSS in the forward orientation. The D segments each had an upstream 12-bp RSS in the reverse orientation and a downstream 12-bp RSS in the forward orientation. The J segments had an upstream 23-bp RSS in the reverse orientation and a downstream splice donor site. The sequences of the 12-bp and 23-bp RSSs were as follows:

12-bp RSS:
[SEQ ID NO: 20]
CACAGTGGTACAGACCAATACAAAAACC
23-bp RSS:
[SEQ ID NO: 19]
CACAGTGGTAGTACTCCACTGTCTGGGTGTACAAAAACC

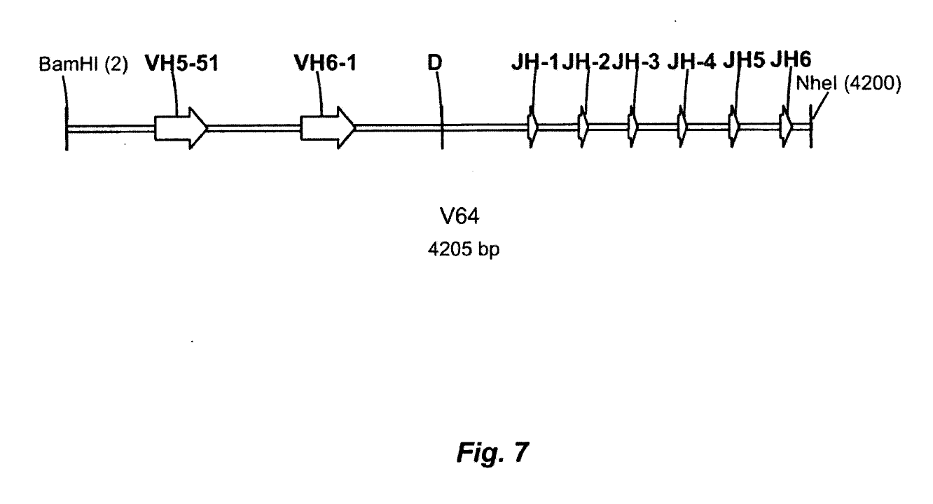

V64 encoded a VDJ heavy chain recombination substrate consisting of two V segments, a single D segment and six J segments (schematic diagram shown in FIG. 7). The sequences of two V64 variants are shown in SEQ ID NO:14 and SEQ ID NO:15, each having a different D segment. In these two variants, each V segment had an upstream SV40 early promoter and a downstream 23-bp RSS in the forward orientation. The D segment had an upstream 12-bp RSS in the reverse orientation and a downstream 12-bp in the forward orientation. The J segments each had an upstream 23-bp RSS in the reverse orientation and a downstream splice donor site. The sequences of the 12-bp and 23-bp RSSs were as follows:

Upstream V64.1 12-bp RSS
SEQ ID NO: 21
CACATAGCAGGAGGGCCTTCACAAAAAGC
Downstream V64.1 12-bp RSS
SEQ ID NO: 22
CACAGTGATGAACCCAGCAGCAAAAACT
Upstream V64.3 12-bp RSS
SEQ ID NO: 23
CACAGTAGGAGGGGCCTTCACAAAAAGC
Downstream V64.3 12-bp RSS
SEQ ID NO: 24
CACAGTGATGAAACTAGCAGCAAAAACT
23-bp RSS (all)
SEQ ID NO: 19
CACAGTGGTAGTACTCCACTGTCTGGGTGTACAAAAACC

Vector V67 encoded a VDJ heavy chain recombination substrate having one V segment, a single D segment and six J segments. The V segment had an upstream SV40 early promoter and a downstream 23-bp RSS in the forward orientation. The D segment had an upstream 12-bp RSS in the reverse orientation and a downstream 12-bp in the forward orientation. The J segments each had an upstream 23-bp RSS in the reverse orientation and a downstream splice donor site. The sequence of the 12-bp and 23-bp RSSs were as follows:

Upstream 12-bp RSS:
[SEQ ID NO: 25]
CACATAGCAGGAGGGCCTTCACAAAAAGC
Downstream 12-bp RSS:
[SEQ ID NO: 26]
CACAGTGATGAACCCAGCAGCAAAAACT
23-bp RSS (all):
[SEQ ID NO: 19]
CACAGTGGTAGTACTCCACTGTCTGGGTGTACAAAAACC

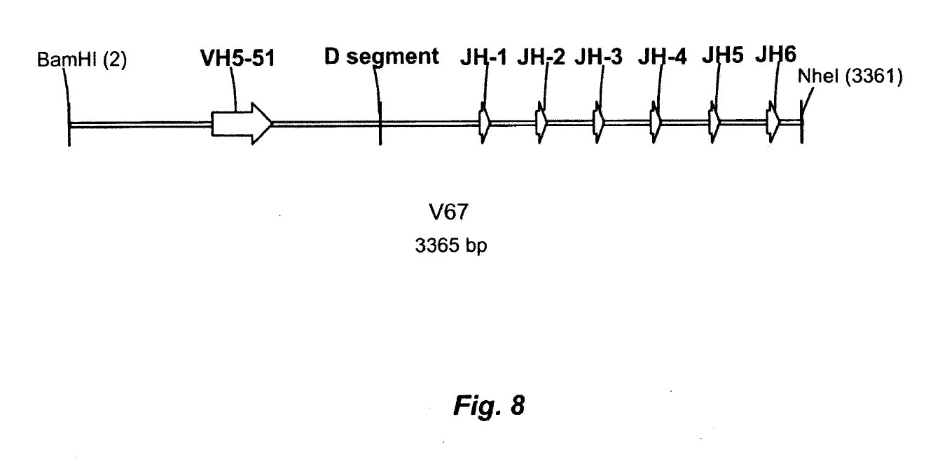

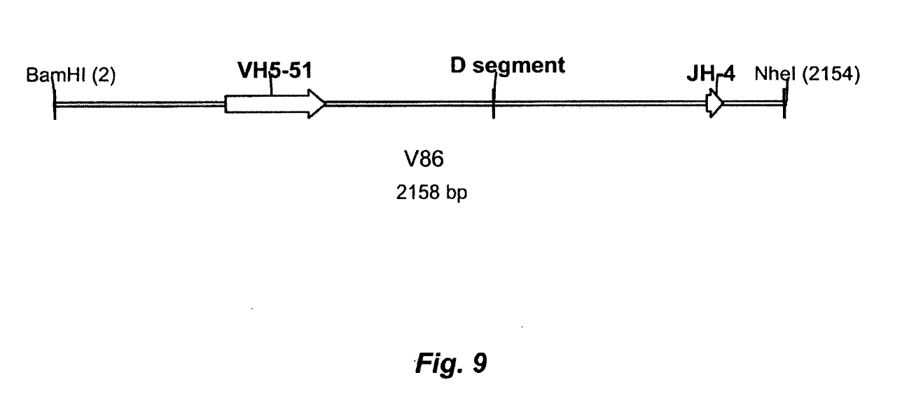

A schematic diagram of V67 is shown in FIG. 8. The sequence is shown in SEQ ID NO:16.

Another antibody generating substrate, V86, encoded a heavy chain recombination substrate having one V segment, one D segment and one J segment. The V segment had an upstream SV40 early promoter and a downstream 23-bp RSS in the forward orientation. The D segment had an upstream 12-bp RSS in the reverse orientation and a downstream 12-bp in the forward orientation. The J segment had an upstream 23-bp RSS in the reverse orientation and a downstream splice donor site. The sequences of the 12-bp and 23-bp RSSs were as follows:

Upstream 12-bp RSS:
SEQ ID NO: 27
CACATAGCAGGAGGGCCTTCACAAAAAGC
Downstream 12-bp RSS:
SEQ ID NO: 28
CACAGTGATGAACCCAGCAGCAAAAACT

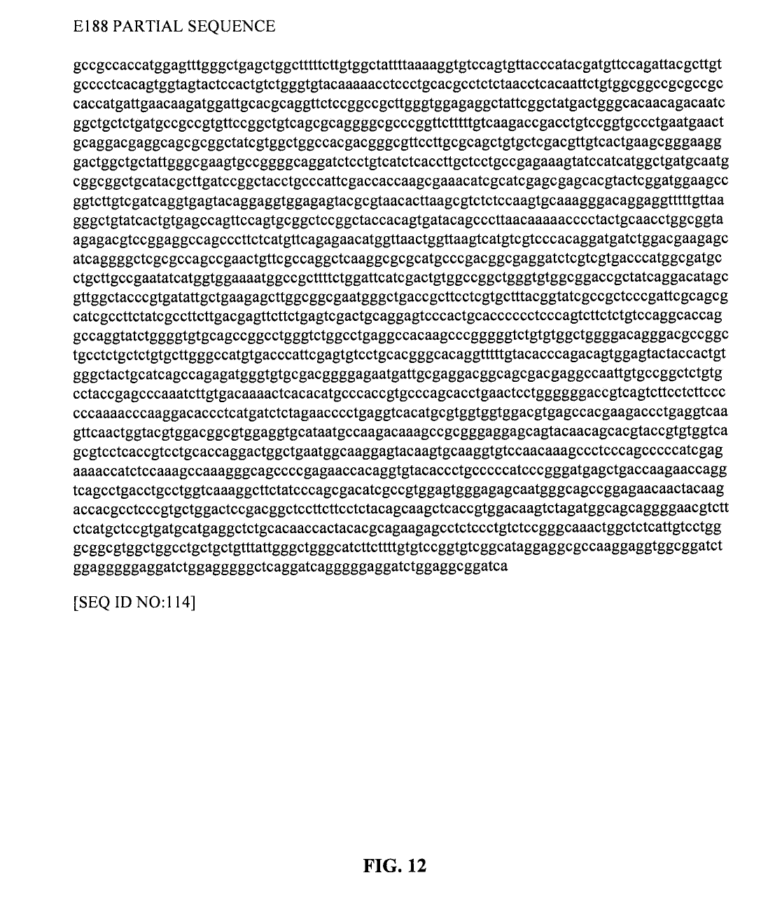

A schematic diagram of V86 is shown in FIG. 12. The V86 sequence is shown in SEQ ID NO:17. The antibody generation vectors V67 and V86 were shown to generate a membrane expressed antibody when co-transfected with RAG-1, RAG-2 and a human kappa chain antibody.

Briefly, 293-HEK cells were split 1:4 into 10 cm² dishes 24 hours prior to transfection. Transfection was performed with Lipofectamine™ 2000 (Invitrogen, cat #11668-019) per the manufacturer's suggested protocol. The heavy chain recombining vector (12.0 μg), V67 or V68, was transfected with an equal mass of DNA representing a 1:1:1:1 ratio of RAG-1, RAG-2, ITS-4 and V25, respectively. V25 was included as an internal control for recombination. In addition to the heavy chain recombining substrates (V67 or V86), ITS-6 was also transfected as a positive control. 72 hours post-transfection, media were aspirated and the cells were washed 1× with 5 ml of PBS and then detached using 1 ml of 0.1× trypsin for 5 minutes at room temperature. Following this 5-minute incubation, the trypsin was neutralized with 8 ml of DMEM supplemented with 10% FBS. The cells were then transferred to a 15 ml conical vial and centrifuged at approximately 800 g for 5 minutes. Media were then aspirated and the cells were resuspended in 500 ul of PBS containing 2% FBS (staining buffer) transferred to a 1.5 ml microcentrifuge tube and centrifuged for an additional 2 minutes at 3000 rpm. Media were then aspirated and the cells were resuspended in 200 μl of staining buffer with 1:200 dilution of a Goat-anti-Human IgG H+ L-PE conjugated polyclonal antibody (Cedarlane, Burlington, N.C., Cat. #109-115-098, stock concentration 0.5 μg/ml). The cells were incubated on ice for 1 hour and then washed 2 times with 200 μl PBS and finally resuspended into 100 μl of staining buffer. Positive cells were visualized by fluorescence microscopy and quantified using flow cytometry (Table 3).

TABLE 3
Immunocytofluorimetric Detection of Surface
Ig Positive (sIg+) Transfectants

| | | Surface Ig Positive Events | |
|---|---|---|---|
| Vector Name | Description | # of Events | % Positive |
| V2 | Empty vector | 476 | 0.05% |
| ITS-6 | Recombined Heavy Chain | 26824 | 27.82% |
| V67 | 1V-1D-6J substrate | 1486 | 0.15% |
| V86 | 1V-1D-1J substrate | 1074 | 0.11% |

Transfection with the control ITS-6 vector showed that a large fraction of cells expressed membrane human IgG1. Transfection with V67 and V86 each showed a low percentage of positive cells. Although these frequencies were relatively low, fluorescent cells were visualized under the microscope for each vector (V67 and V86).

In a separate experiment, stable cell lines were generated using the V64.1 and V64.3 substrates (described above). HEK-293H cells were transfected with equal amounts of five expression plasmids using Lipofectamine 2000 (Invitrogen, Cat. #11668-019) as per the manufacturer's suggested protocol. The vectors included: 1) RAG1, 2) RAG2, 3) V64, (2V-1 D-6J), heavy chain VDJ substrate, 4) a fully recombined antibody light chain (ITS-4) and 5) a vector containing the puromycin resistance gene. Forty-eight hours post-transfection, cells were selected using 1.0 ug/ml puromycin for 2 weeks. Puromycin resistant clones were then plucked and expanded into 6 well dishes. Once the cells had achieved confluence, media were aspirated and the cells were washed 1× with 2 ml of PBS and then detached using 0.5 ml of 0.1× trypsin for 5 minutes at room temperature. Following the 5 minute incubation the trypsin was neutralized with 2 ml of DMEM supplemented with 10% FBS. Half of the cells were then transferred to a 1.5 ml microcentrifuge tube and spun at 3000 rpm for 2 minutes. Media were then aspirated and the cells were resuspended in 200 ul of PBS containing 2% FBS (staining buffer) with 1:200 dilution of a Goat anti-Human IgG H+L-PE conjugated polyclonal antibody (Cedarlane, Cat #109-115-098, stock concentration 0.5 ug/ml). The cells were incubated at 4 degree Celsius for 1 hr and then washed 2 times with 150 ul PBS, then resuspended into 100 ul of staining buffer. Positive cells were visualized using fluorescent microscopy and quantified using flow cytometry (Table 4).

The transfection resulted in host cells containing chromosomally integrated, fully assembled (e.g., rearranged relative to the germline) and functional immunoglobulin light chain gene that was constitutively expressed (ITS-4). The stable cell line also expressed RAG-1 and RAG-2 and a heavy chain diversity generating vector(s) encoding an Ig fusion protein having a membrane anchor domain as described herein (V64). The light chain was secreted and was not found on the cell surface unless associated with a membrane-associating heavy chain. Cells that did not produce Ig heavy chain gene VDJ events, or that generated out-of-frame products, were not able to generate a heavy chain. Cells that did produce a functionally rearranged heavy chain gene were able to assemble the expressed heavy chain in association with the light chain and so generated a membrane bound antibody, due to the membrane anchoring domains included in the heavy chain diversity generating vector. Clones of 293 cells harboring integrated V64 (1V-1 D-6J) VDJ substrates were analyzed by FACS (10,000 cells analyzed). A number of clones were identified that expressed human IgG on the cell surface of a significant number of cells (Table 5). Immunofluorescence microscopy readily permitted visualization of cells with fluorescently stained human IgG on their surfaces.

TABLE 4
Immunocytofluorimetric Detection of Surface Ig Positive (sIg+)
Transfectants by Fluorescence Activated Cell Sorter (FACS) Analysis

| | | | % Surface Ig |
|---|---|---|---|
| Filename | Clone ID | Description | Positive Cells |
| Specimen_001_1.fcs | 1 | V64.3 clone 1 | 0.2% |
| Specimen_001_4_003.fcs | 7 | V64.3 clone 7 | 5.4% |
| Specimen_001_4_012.fcs | 16 | V64.1 clone 8 | 8.2% |
| Specimen_001_4_021.fcs | 25 | V64.1 clone 17 | 10.5% |
| Specimen_001_4_023.fcs | 27 | V64.1 clone 19 | 3.1% |

With such demonstrated expression of the antibody product of VDJ recombination on the cell surface, antigen-binding or anti-Ig binding assays can be performed to identify cells expressing Ig heavy chains having desired binding properties.

It should be appreciated that in related alternative embodiments, the above described process can be conducted with a stably integrated immunoglobulin heavy chain gene in the host cell, into which are introduced light chain diversity generating vectors assembled as described herein. A rearranged heavy chain gene recovered from a host cell expressing an immunoglobulin having desired binding properties and identified as described above in this Example, can be integrated into a host cell and subsequently a light chain diversity generating vector can be used. For example and according to non-limiting theory, by this approach both the heavy chain and the light chain CDR3s are selected for a desired binding activity (e.g., specific binding to a desired antigen) to generate high affinity antibodies.

### Example 5

### Diversifying Both Heavy and Light Chains in a Single Host Cell

This Example describes introducing Ig heavy and light chain diversification constructs into the same host cell. In order to avoid the recombination signals from the two constructs being utilized inappropriately (e.g., V_{H} to J_{L} etc.) it is preferred to have the constructs introduced sequentially so that they integrate into different chromosomes. A trans-chromosomal recombination event between the two constructs is not impossible but kinetically the intrachromosomal recombination event is favored. At least one D segment gene is present on each nucleic acid construct for generating immunoglobulin diversity, so that all V and J gene segments (both heavy chain and light chain) contain the same RSS spacer size (i.e., 12 or 23 nucleotide signals as described above) whilst the D segment gene contains the functionally complementary RSS spacer size (i.e., 23 nt if V and J use 12 nt; 12 nt if V and J use 23 nt); this configuration precludes direct V to J recombination events.

Including the D segment gene on the Ig light chain diversity construct promotes the generation of a diverse light chain repertoire. Again, because of the 12/23 rule it prevents direct V to J recombination. In the in vitro system, which does not contain the regulatory controls found in vivo that terminate recombination following the successful completion of a functional light chain gene assembly, multiple rounds of light chain recombination transpire until either the expression of the recombinase is stopped or all the light chain V and J gene segments are consumed. In either event significant biases are observed and proximal V and J genes (e.g., V region genes further from the 5′ terminus and J segment genes further from the 3′ terminus) are more frequently deleted and under-utilized.

The tripartite V-D-J assembly process for Ig light chain gene recombination promotes an unprecedentedly diverse light chain repertoire. The D segment encoding polynucleotides of the D segment gene(s) include natural D segment encoding gene sequences found in the human genome and/or artificial D segment encoding sequences.

In a preferred embodiment artificial D segment genes having D segment encoding polynucleotide sequences with between 1 and 6 nucleotides predominantly containing a “G” or “C” are included so as to mimic the biased addition of TdT. Because N nucleotide addition is generally lower at the light chain locus and deletions occur at both the 5′ and 3′ ends of the D segment encoding sequence, the remaining G/C nucleotides are functionally equivalent to TdT additions and provide additional diversity at the light chain locus. The products from larger species of such D-like segments with high G/C content thus represent the fucntional equivalents of larger N nucleotide insertions.

Although an artificial D segment encoding sequence having one or only a few nucleotides (e.g., 2, 3, 4, 5) is likely on a probabilistic basis to be eliminated by deletion accompanying recombination, low probability successful recombination events that utilize the D segment encoding sequence enhance light chain sequence diversity, and deletional events that eliminate the D segment still contribute to reduced positional (e.g., 5′ or 3′) bias in the usage of light chain V and J gene segments in productive recombination.

Another nucleic acid composition for generating Ig structural diversity includes three D segment genes on a light chain diversity generating construct: 3′ to the V region genes is a first D segment encoding gene having the nucleotide sequence 5′-(GCGC)-3′ situated between a first D segment upstream RSS and a first D segment downstream RSS; downstream from the first D segment encoding gene is a second D segment encoding gene having a single “G” nucleotide situated between a second D segment upstream RSS and a second D segment downstream RSS; downstream from the second D segment encoding gene is a third D segment encoding gene that is proximal to a J segment gene and that has the nucleotide sequence 5′-(GGCGCC)-3′ situated between a third D segment upstream RSS and a third D segment downstream RSS. In this exemplary light chain diversity-generating composition, D segment encoding sequences are separated by sequences that are also found separating D segment genes of the heavy chain locus in the human genome.

### Example 6

### Preparation of Constructs for Introducing Sequence Diversity into an Avimer

A domain or avimer-encoding DNA sequences were generated by gene synthesis by GeneArt® (Invitrogen, Carlsbad, Calif.). The sequences were codon-optimized and included RSSs in the appropriate positions, an IgG1 hinge region, CH2, CH3, a 5′ hemaglutin (HA) tag, a PDGFR transmembrane domain sequence and a selectable marker, as detailed in Tables 5 and 6 below.

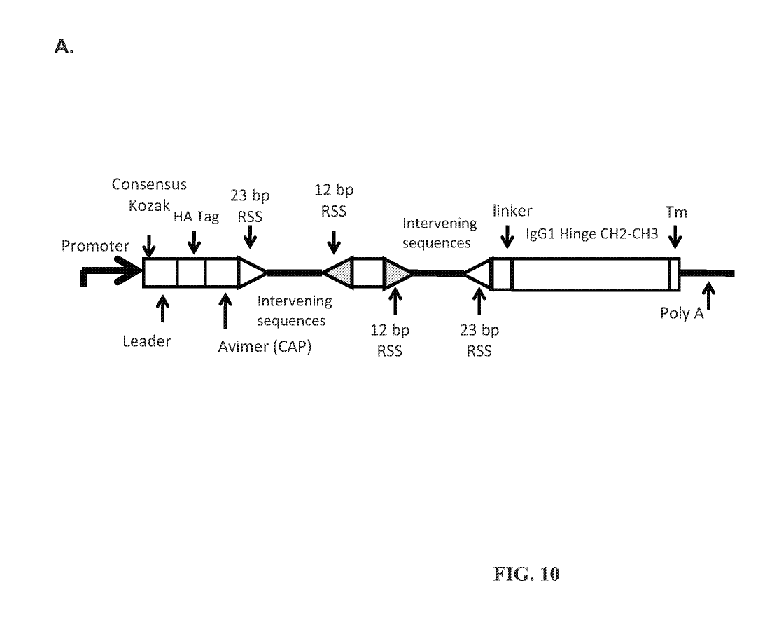

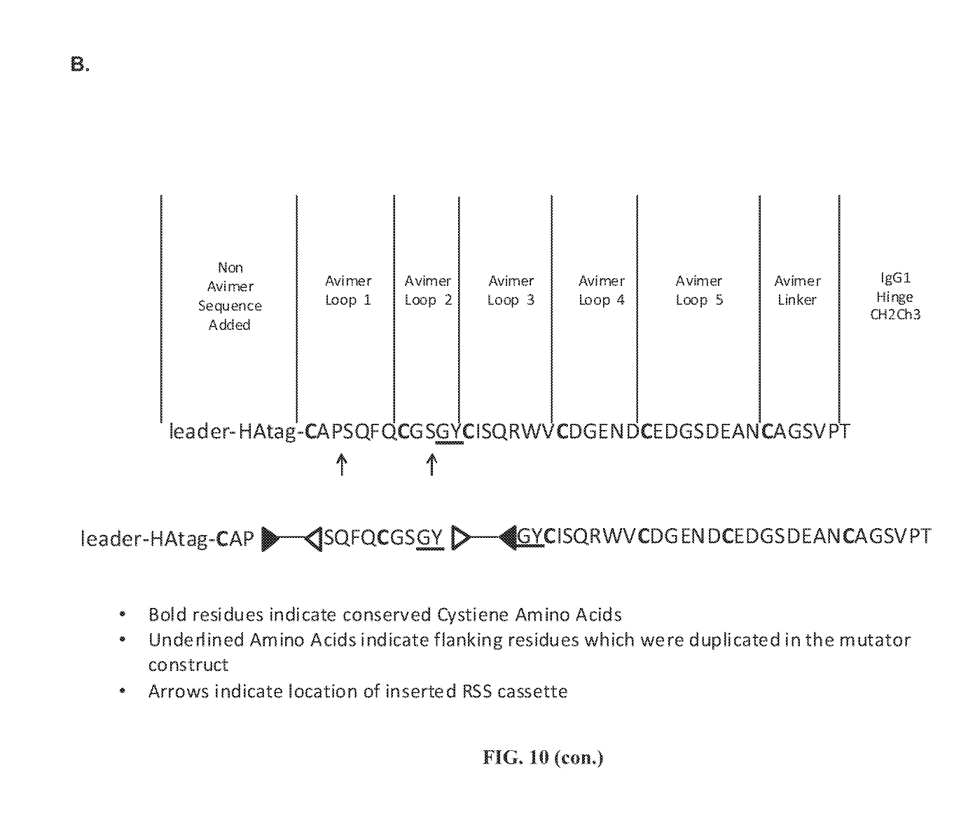

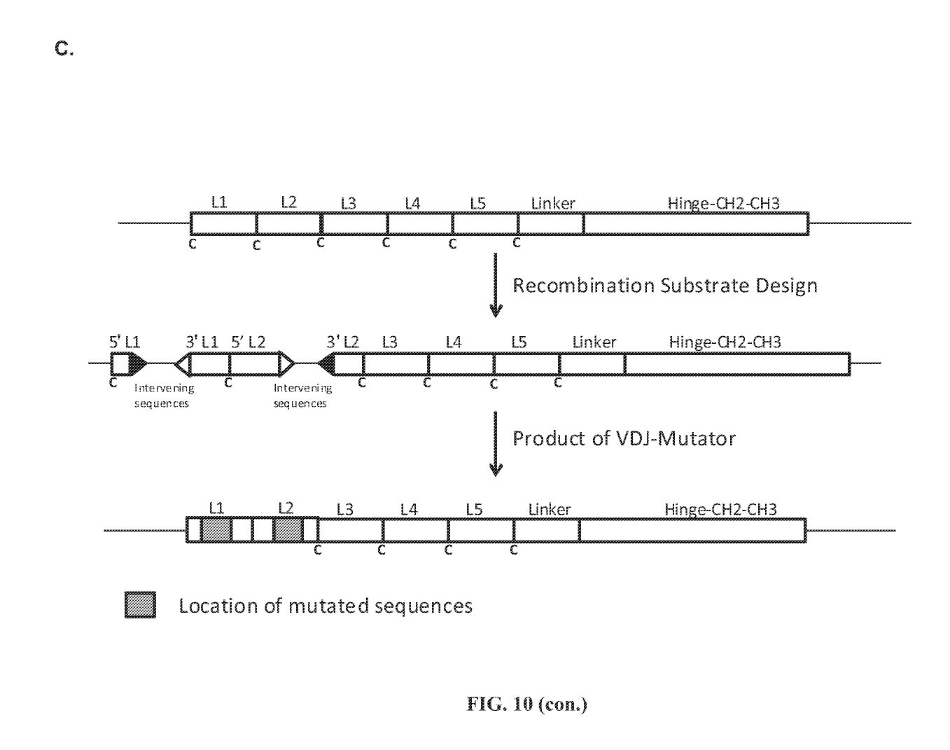

E188 is a single A domain avimer construct and includes a pair of RSSs introduced into loop 1 of the construct and a pair of RSSs introduced into loop 2 of the construct together with flanking sequences encoding GY amino acid residues, which were selected to be a duplication of the naturally occurring residues, but could also have been non-endogenous sequences (see FIG. 10A-C).

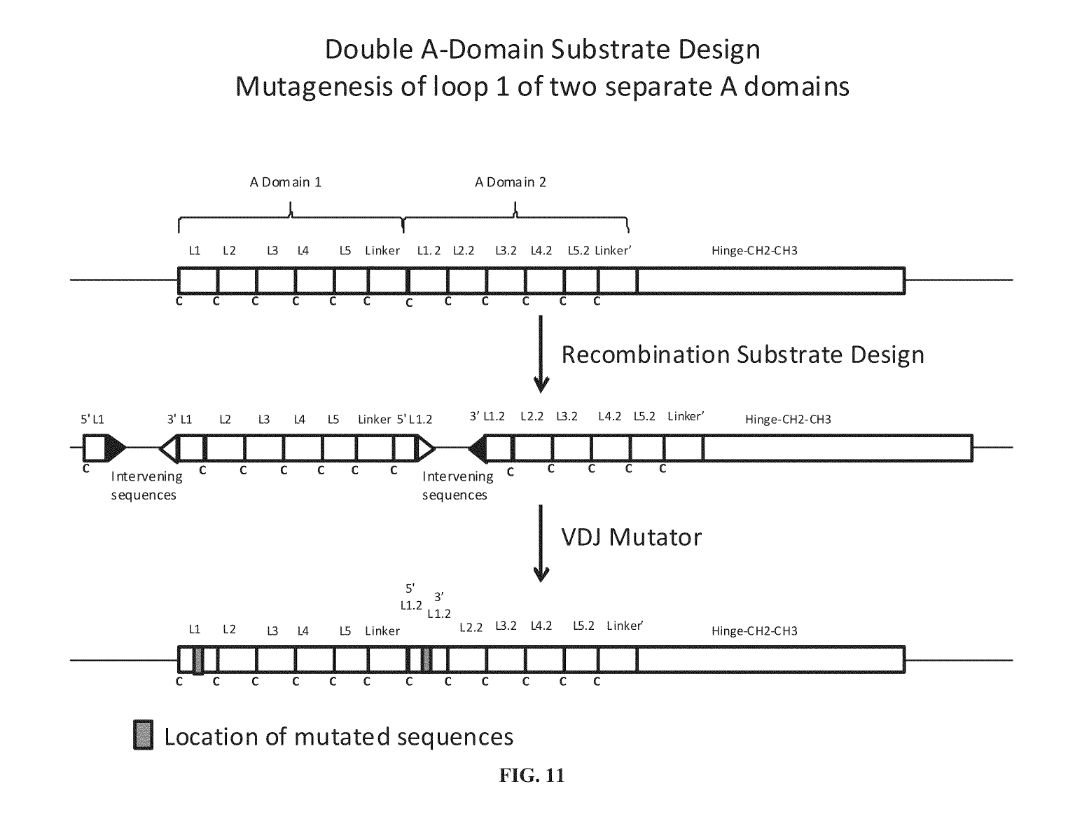

E189 is a double A domain avimer construct and includes a pair of RSSs in each loop 1 of the construct (see FIG. 11). E189 also includes stop codons in other reading frames in the 3′ loop 1 to 5′ loop 1.2 region, but does not include flanking sequences.

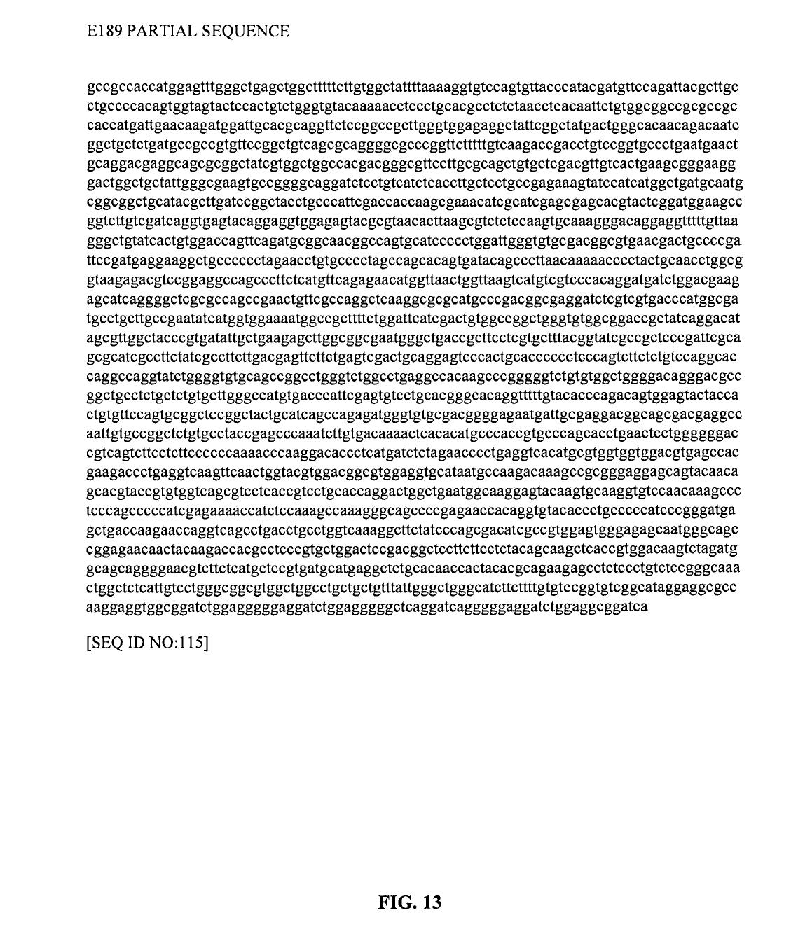

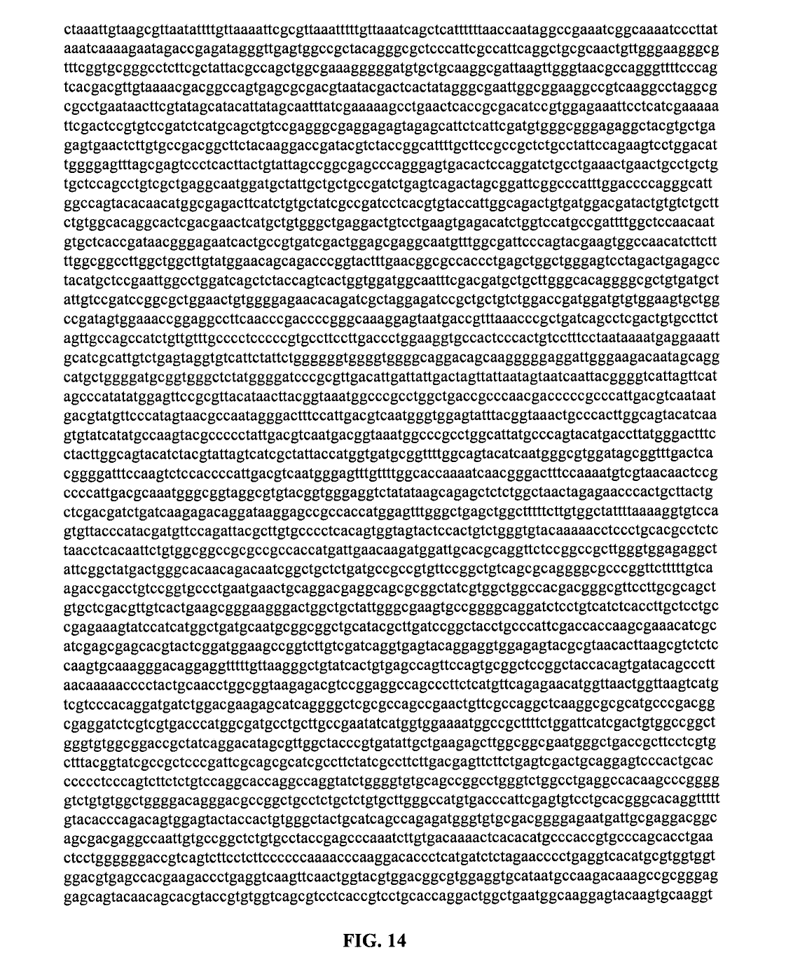

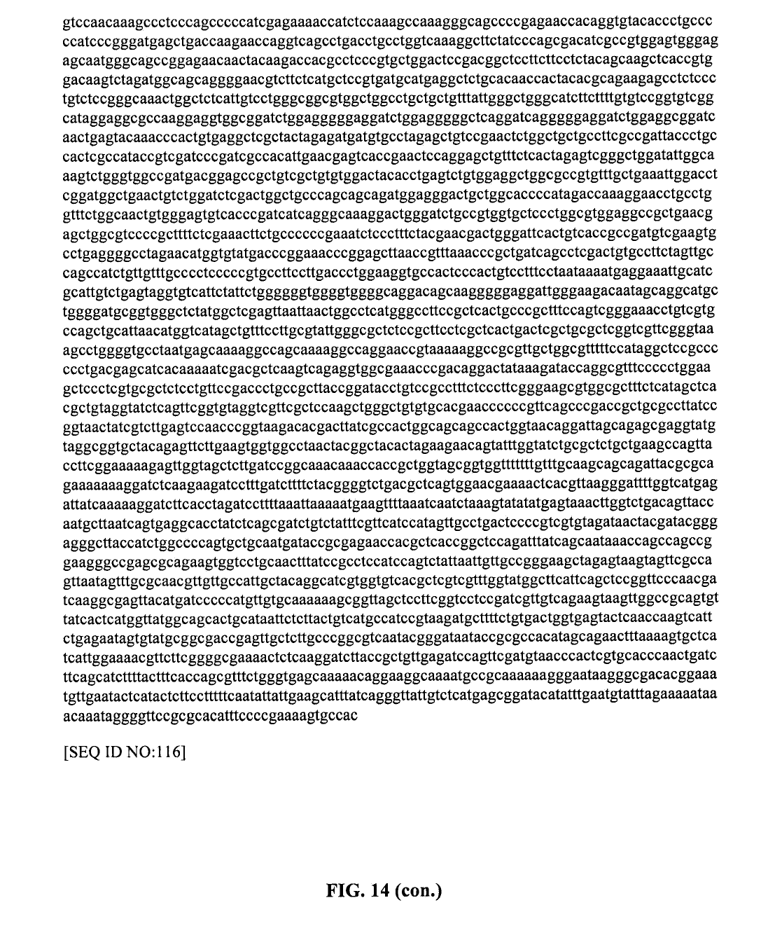

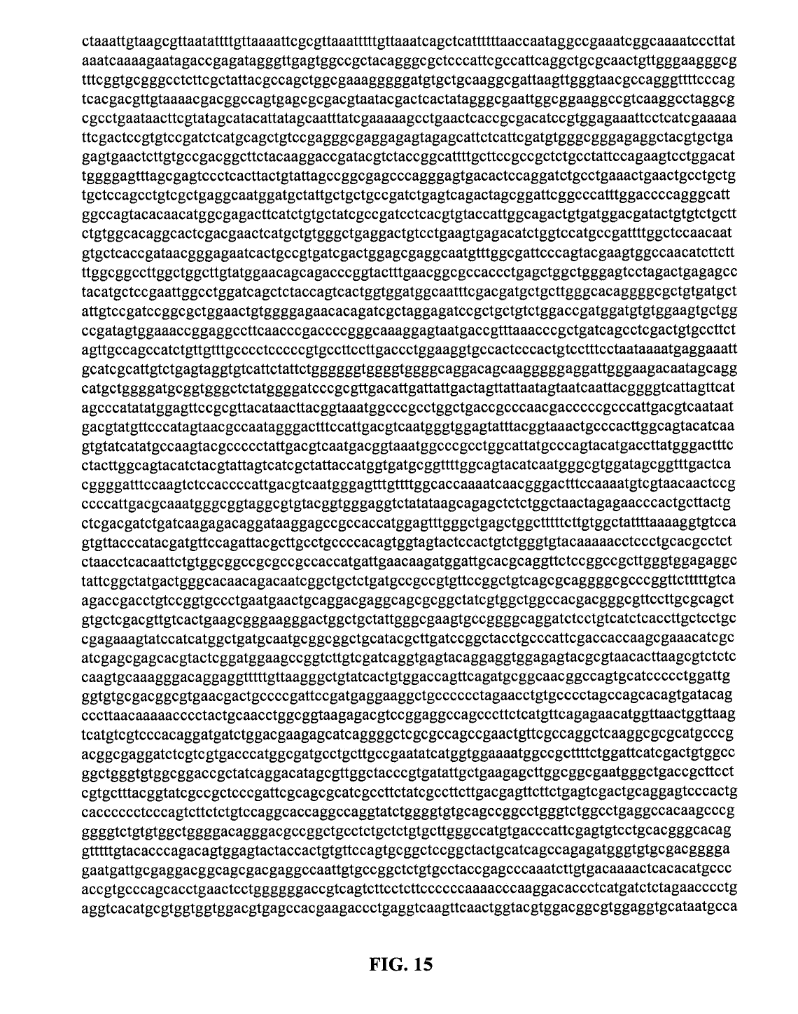

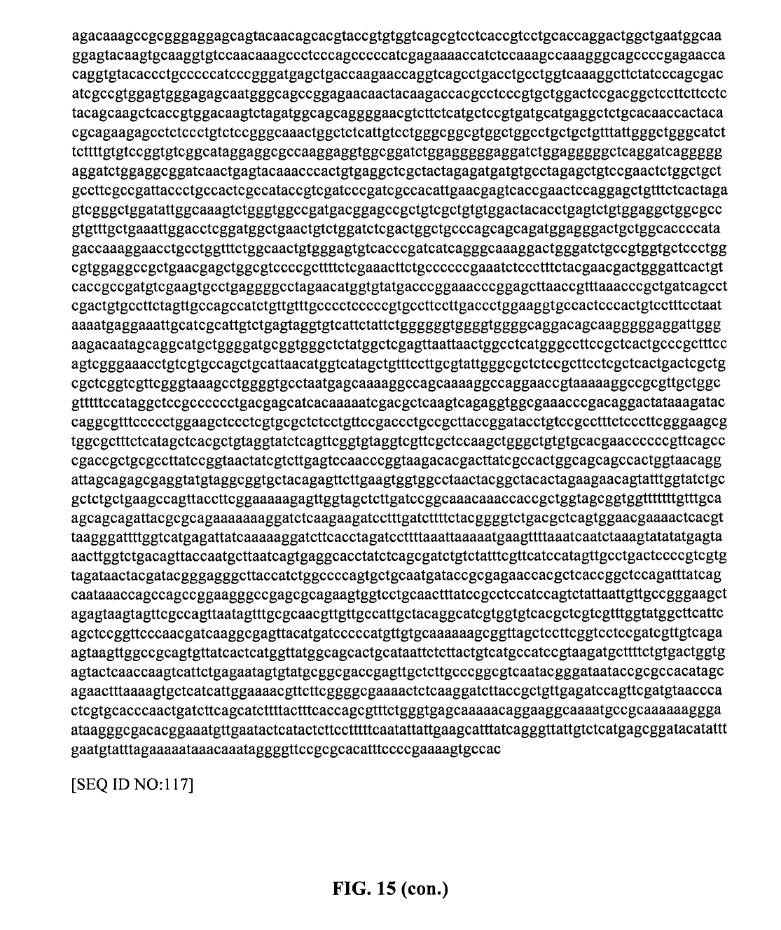

Portions of the E188 and E189 sequences are shown in FIG. 12 [SEQ ID NO:114] and FIG. 13 [SEQ ID NO:115], respectively. The complete vector sequences are provided in FIG. 14 [SEQ ID NO:116] and FIG. 15 [SEQ ID NO:117], respectively.

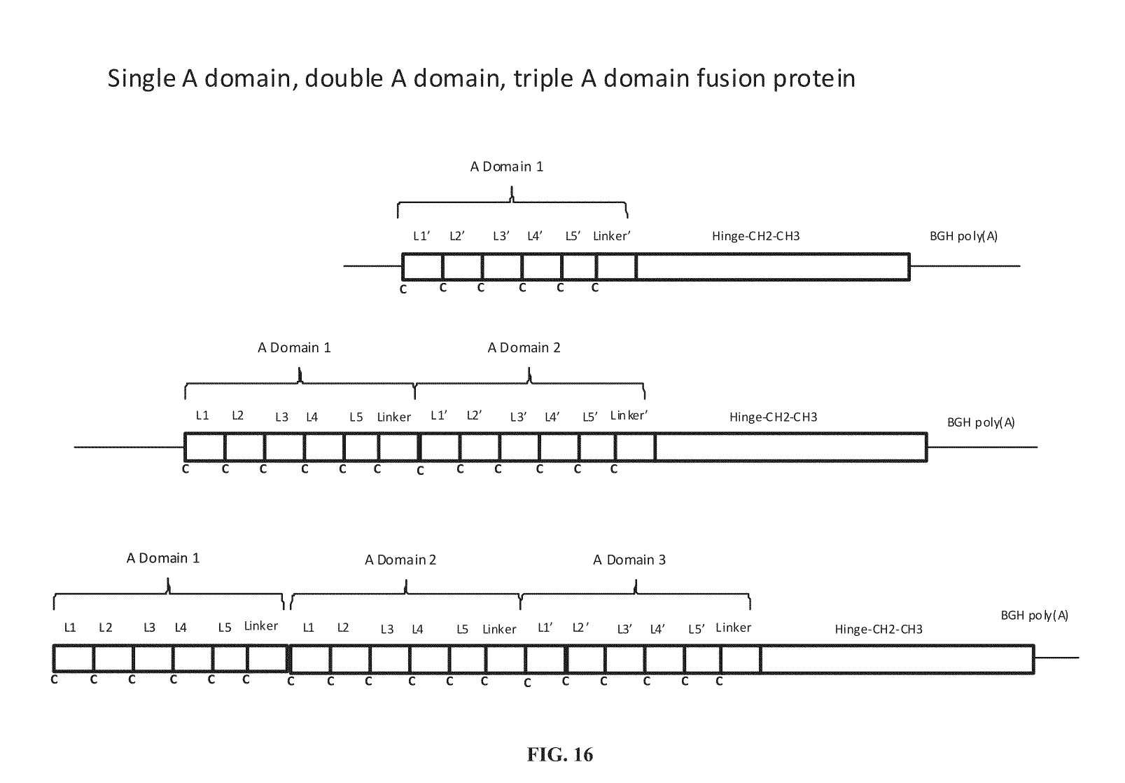

Multiple A domain avimers can also be constructed (see FIG. 16).

TABLE 5
Sequence Annotation for [SEQ ID NO: 114]

| | |
|---|---|
| Leader | 10-66 |
| HA-tag | 67-93 |
| Coding sequences 5′ loop 1 | 94-102 |
| Inserted flanking sequence | NA |
| 23 bp RSS (>) | 103-141 |
| Intervening sequence | 142-722 |
| 12 bp RSS (<) | 723-250 |
| Inserted flanking sequence | NA |
| Coding intervening sequence 3′ loop | 751-771 |
| 1/5′ loop 2 | |
| Inserted flanking sequence (GGCTAC) | 772-777 |
| 12 bp RSS (>) | 778-805 |
| Intervening sequence | 806-1429 |
| 23 bp RSS (<) | 1430-1468 |
| Inserted flanking sequence | NA |
| 3′ loop 2-loop 5 | 1469-1501 |
| Avimer linker | 1502-1561 |
| IgG1 hinge CH2-CH3 | 1562-2257 |
| Transmembrane sequence | 2258-2425 |

TABLE 6
Sequence Annotation for [SEQ ID NO: 115]

| | |
|---|---|
| Leader | 10-66 |
| HA-tag | 67-93 |
| Coding sequences 5′ loop 1 | 94-102 |
| Inserted flanking sequence | NA |
| 23bp RSS (>) | 103-141 |
| Intervening sequence | 142-722 |
| 12bp RSS (<) | 723-250 |
| Inserted flanking sequence | NA |
| Coding sequence 3′ loop 1- loop 5 linker 5′ loop 1.2 | 751-870 |
| Inserted flanking sequence | NA |
| 12bp RSS (>) | 871-898 |
| Intervening sequence | 899-1522 |
| 23bp RSS (<) | 1523-1561 |
| Inserted flanking sequence | NA |
| Coding sequences 3′ loop 1.2 - loop 5.2 | 1562-1609 |
| Avimer linker | 1610-1669 |
| IgG1 hinge CH2-CH3 | 1670-2365 |
| Transmembrane sequence | 2366-2533 |

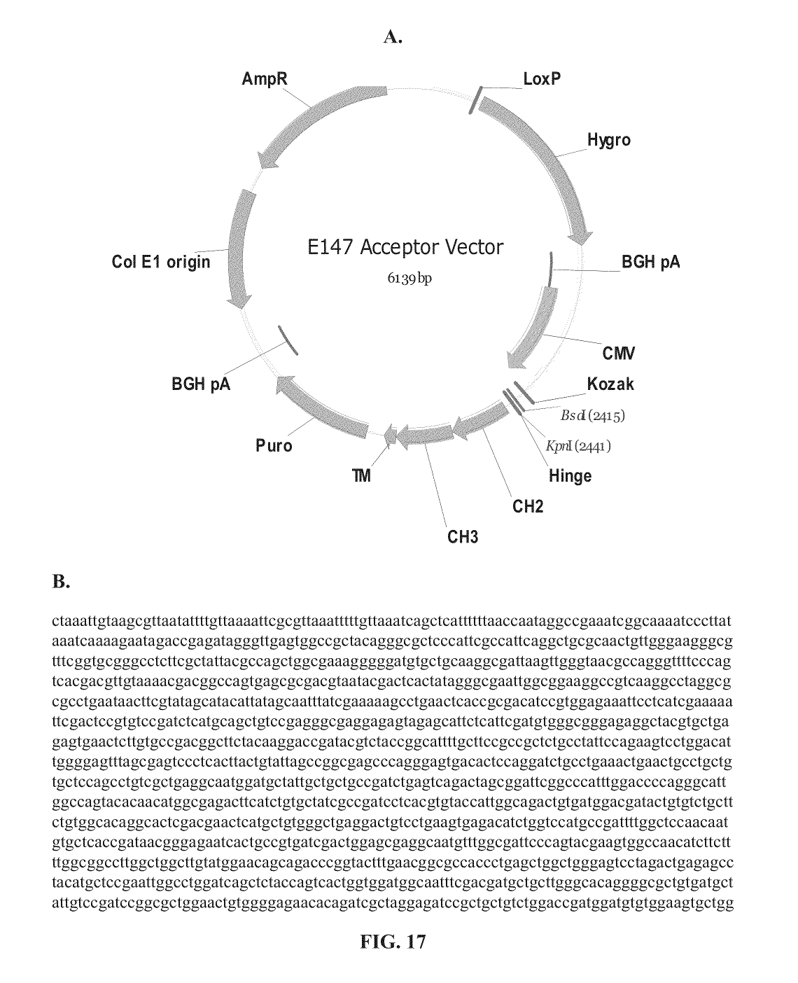

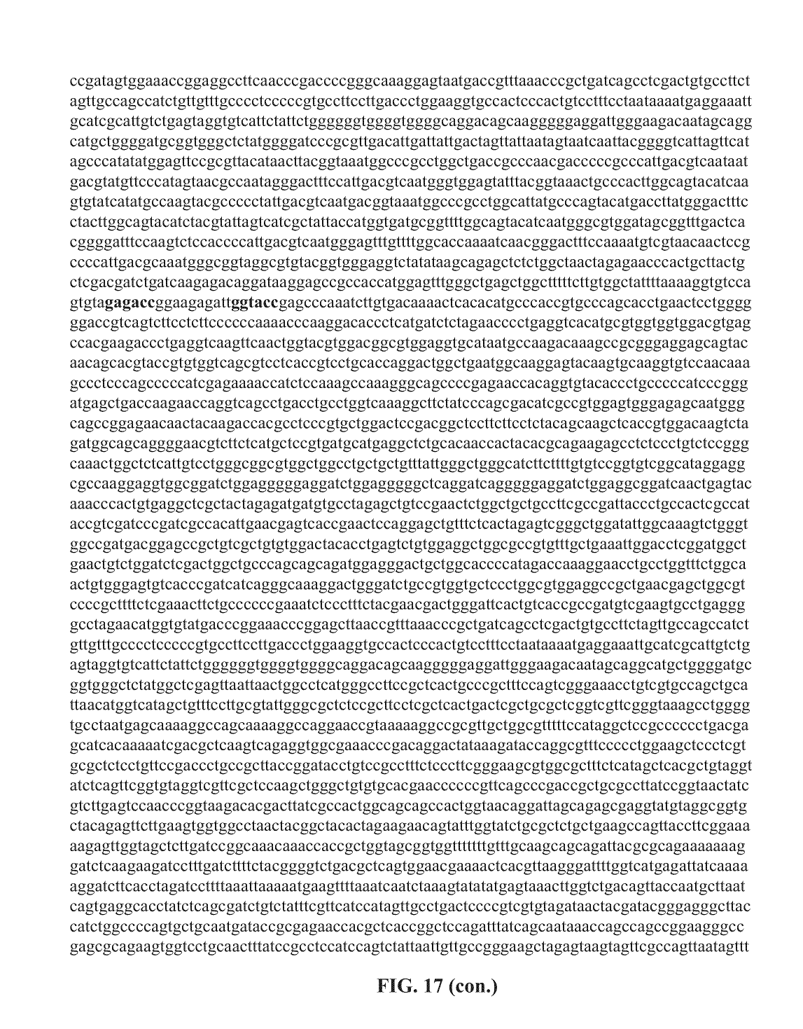

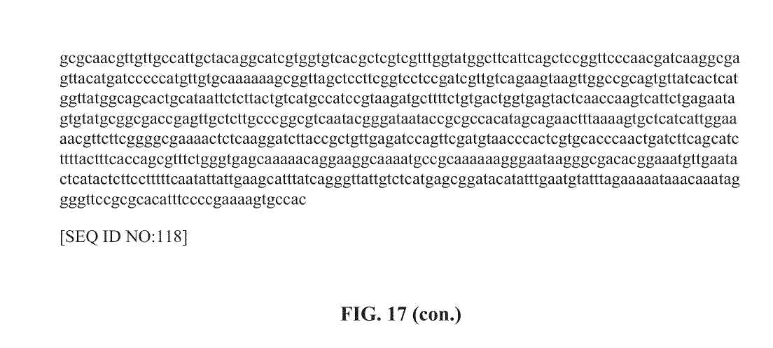

The synthesized DNA was cloned into a modified pcDNA (Invitrogen, Carlsbad, Calif.) that contains a consensus Kozak sequence and a mammalian leader signal sequence (see FIG. 17) for efficient secretion or surface expression of the recombined avimers. The modified pcDNA acceptor vector allows for cloning of the avimer construct so that the 3′ end is fused to the Fc portion of human IgG1 followed by a PDGFR transmembrane domain and selectable marker such that the recombined molecules are surface expressed and can be selected for in-frame products. The nucleotide sequences for the IgG hinge through CH₃ sequences and a transmembrane domain are shown in FIG. 17B [SEQ ID NO:118]. The avimer scaffold was cloned at the KpnI site (bolded in FIG. 17B), which translates as a Gly-Thr prior to the hinge sequences of IgG1.

### Example 7

### Generation of Surface Expressed Avimer Mutants

Avimer vectors containing E188 prepared as described in Example 6 were transfected into a recombination competent cell line and stable neomycin integrants were generated. The sequences of the expressed avimer mutants were obtained as described in Example 9 below.

### Example 8

### Generation of Libraries of Surface Expressed Avimer Mutants

Avimer vectors containing E188 prepared as described in Example 6 were stably integrated into a recombination competent cell line. Stable integrants were expanded and then transfected with plasmids expressing RAG1/RAG2/TdT. The transfection was carried out using 1×107 stable integrants transfected with 8 ug each of RAG1, RAG2 and TdT expression vectors using a 3:1 ratio of linear PEI (1 mg/ml) to DNA.

RAG1/RAG2/TdT treated cells were then stained using anti-IgG Fc to confirm surface expression of the recombined avimer molecules. Approximately 1×106 cells were stained with 1 ug/ml Biotin conjugated anti-human IgG Fc (Jackson Laboratories) for 30 min. The cells were then washed twice and stained with streptavidin-conjugated Alexa-647 for 30 min. Samples were subsequently washed twice, resuspended in 300 ul of PBS and analyzed using flow cytometry. The recombined population was shown to have high uniform expression. The sequences of the expressed avimer mutants were obtained as described in Example 9 below.

### Example 9

### Sequence Analysis of Avimer Mutants (Single a Domain)

RNA samples obtained from FACS sorted cells (Example 8) were used for sequence analysis of the expressed avimer variants. mRNA from approximately 106 recombined cells was purified using Qiagen RNeasy RNA purification kit as per the manufacturer's recommendations. cDNA synthesis was carried out using Superscript enzyme (Invitrogen, Carlsbad, Calif.) as per the manufacturer's recommended protocol and primer MG59 (sequence 5′-TCTTGGCATTATGCACCTCCACGCCGTCC-3′ [SEQ ID NO:119]).

The cDNA was then used as a temple and amplified using primer MG301 (sequence 5′-GAGAGAGATTGGTCTCGAGAACCCACTGCTTACTGCTCGACGATCTGAT-3′ [SEQ ID NO:120]), which anneals in the 5′ UTR region, and primer MG58 (sequence 5′-GTCTTCGTGGCTCACGTCCACCACCACGCA-3′ [SEQ ID NO:121]), which anneals internal to the MG59 primer used in the RT reaction.

The amplified product was purified using a Qiagen PCR clean up kit as per the manufacturer's recommended protocol and eluted into 35 ul of water. The purified PCR product was then digested with Bsal (NEB) and cloned into the modified pcDNA acceptor vector (Invitrogen, Carlsbad, Calif.) with corresponding compatible ends. Plasmid DNA from E. coli cultures was purified using Qiagen Miniprep kit and avimer sequences were analyzed using primer MG60 (sequence 5′-CTGACCTGGTTCTTGGTCAGCTCATCCCG-3′ [SEQ ID NO:122]).

The results are presented in Tables 7 and 8 below.

TABLE 7
Nucleotide Sequence Analysis Of Single A Domain Avimer Variants

| Mutant | L1 5′ | L1 Additions | L1 3′ | L2 5′ | L2 Additions | L2 3′ |
|---|---|---|---|---|---|---|
| # | Deletions | [SEQ ID NO] | Deletions | Deletions | [SEQ ID NO] | Deletions |
| 1 | −1 | | −2 | 0 | GA | −2 |
| 2 | 0 | AGGGCCAAGA [123] | −15 | −7 | TGGGGTTAAGCCTC [124] | −2 |
| 3 | −1 | GAG | −2 | 0 | | 0 |
| 4 | 0 | C | −1 | 0 | GGG | −6 |
| 5 | −2 | TAGGGGGTTCCAGT | −13 | −2 | GAG | 0 |
| | | [125] | | | | |
| 6 | 0 | AGAA | −3 | −12 | CCCTCCGTCCTACCTC | −2 |
| | | | | | [126] | |
| 7 | 0 | AGTGGGGAT | 0 | −12 | C | −4 |
| 8 | −1 | CCC | −6 | −14 | TCCAGTGCGGCTCCGGGA | −24 |
| | | | | | [127] | |
| 9 | −1 | CCT | −2 | −2 | TC | 0 |
| 10 | −2 | T | 0 | −2 | | −3 |
| 11 | −8 | TCC | −4 | −4 | CTACA | −4 |
| 12 | 0 | AC | −3 | −4 | CG | −3 |
| 13 | 0 | AGAAGG | −3 | 0 | | −3 |
| 14 | −3 | TTATTA | −1 | 0 | | −2 |
| 15 | −2 | AAGAC | −12 | 0 | GTC | −2 |
| 16 | 0 | CC | −5 | 0 | | −6 |
| 17 | −1 | CTC | −3 | −13 | | −4 |
| 18 | 0 | AGG | 0 | −23 | GGAGCCGCACTGGAACT | 0 |
| | | | | | [128] | |
| 19 | 0 | | −1 | −2 | | −6 |
| 20 | 0 | CG | −5 | −2 | CT | −6 |
| 21 | 0 | AGAC | −1 | −2 | TCCC | −2 |

TABLE 8
Amino Acid Sequence Analysis Of Single A Domain Avimer Variants

| | | | | Total aa Length |
|---|---|---|---|---|
| Mutant | Loop 1 (5′) | Loop 1 (3′)/Loop2 (5′) | Loop 2 (3′)and loop 3 | (from CAP to |
| # | [SEQ ID NO] | [SEQ ID NO] | [SEQ ID NO] | GYC) |
| Parent | DYACAP [129] | SQFQCGSGY [130] | GYCISQRWVCD [131] | 15 |
| 1 | DYA | FQFQCGSGYN [132] | CISQRWVCD [133] | 10 |
| 2 | DYACAP [129] | TSSSAAPAY [134] | CISQRWVCD [133] | 13 |
| 3 | DYACAP [129] | RRQFQCGSGY [135] | YCISQRWVCD [136] | 14 |
| 4 | DYACA | LLASSSAAPAT [137] | YCISQRWVCD [136] | 13 |
| 5 | DYACA | QDAAPATS [138] | YCISQRWVCD [136] | 13 |
| 6 | DYACAP [129] | PQFQCGSGY [139] | CISQRWVCD [133] | 13 |
| 7 | DYACAP [129] | SSSSD [140] | CISQRWVCD [133] | 13 |
| 8 | DYACAP [129] | RSRSRTGT [141] | GYCISQRWVCD [131] | 15 |
| 9 | DYACAP [129] | ASSSAAPA [142] | CISQRWVCD [133] | 13 |
| 10 | DYACAP [129] | RFQCGSGS [143] | CISQRWVCD [133] | 13 |
| 11 | DYACAP [129] | RRQFQCGSGFP [144] | YCISQRWVCD [136] | 14 |
| 12 | DYACAP [129] | QFQCGSGYD [145] | YCISQRWVCD [136] | 14 |
| 13 | DYACAP [129] | RAKRLWGAS [146] | YCISQRWVCD [136] | 14 |
| 14 | DYACAP [129] | SQFQCGSGY [147] | GYCISQRWVCD [131] | 15 |
| 15 | DYACAP [129] | RQFQCGSGYG [148] | CISQRWVCD [133] | 13 |
| 16 | DYACA | LGGSSAAPAE [149] | GYCISQRWVCD [131] | 14 |
| 17 | DYACAP [129] | RTVPVPLRPTS [150] | YCISQRWVCD [136] | 14 |
| 18 | DYACAP [129] | SGDSQFQCH [151] | CISQRWVCD [133] | 13 |
| 19 | DYACAP [129] | PSSSSAAPG [152] | VCD | 7 |
| 20 | DYACAP | LQFQCGSGF [153] | GYCISQRWVCD [131] | 15 |
| 21 | DYACA | LASSSAAPA [154] | YCISQRWVCD [136] | 13 |

This data indicates that net size of the product is still smaller than the original product indicating that this is a situation in which additional flanking sequences may be beneficial. The data also demonstrated that a large fraction of products used the other reading frames for the RSS flanked cassette and as a result eliminated the cysteine residue. To counter this, an alternative cassette was designed as described in Example 10 below.

### Example 10

### Alternative Construct for Introducing Sequence Diversity into an Avimer

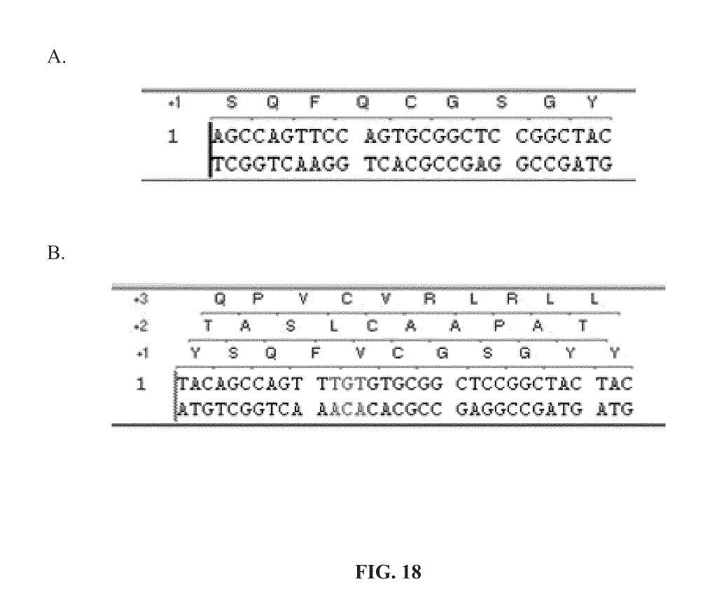

The cassette used in Example 6 (see FIG. 18A) was redesigned as shown in FIG. 18B. The alternate cassette includes as additional flanking sequences, a TAC at both the 5′ end and the 3′ end (adding potential tyrosine if not deleted). The modified cassette also includes nucleotide changes that add cysteines in the other frames to help ensure retention of a cysteine in the final product.

### REFERENCES

Azuma et al., 1976 J Biochem 80:1023; Alt et al., 1984 Embo J 3:1209; Chaney et al., 1986 Somat Cell Mol Genet 12:237; Caporale et al., 1990 Gene 87:285; Alessandrini et al., 1991 Mol Cell Biol 11:2096; Akamatsu et al., 1994 J Immunol 153:4520; Bradshaw et al., 1995 Nucleic Acids Res 23:4850; Connor et al., 1995 J Immunol 155:5268; Corbett et al., 1997 J Mol Biol 270:587; Sauer, 1998 Methods 14:381; Arakawa et al., 2001 BMC Biotechnol 1:7; Choi et al., 2001 Methods Mol Biol 175:57; Chowdhury et al., 2001 Embo J 20:6394; Kaczmarczyk et al., 2001 Nucleic Acids Res 29:E56; Sauer, 2002 Endocrine 19:221; Bruce et al., 2003 Rna 9:1264; Cowell et al., 2003 J Exp Med 197:207; Kondo et al., 2003 Nucleic Acids Res 31:e76; Chatterjee et al., 2004 Nucleic Acids Res 32:5668; Chowdhury et al., 2004 Immunol Rev 200:182; Ciubotaru et al., 2004 Mol Cell Biol 24:8727; Cowell et al., 2004 Immunol Rev 200:57; Arnaout, 2005 BMC Genomics 6:148; Afshar et al., 2006 J Immunol 176:2439; Baird et al., 2006 Rna 12:1755; Browman et al., 2007 Trends Cell Biol 17:394; Chakraborty et al., 2007 Mol Cell 27:842; Chen et al., 2007 Faseb J 21:2931; Ferguson et al., 1986 J Biol Chem 261:14760; Engler et al., 1987 Proc Natl Acad Sci USA 84:4949; Galli et al., 1988 Proc Natl Acad Sci USA 85:2439; Ferrier et al., 1990 Embo J 9:117; Gnirke et al., 1991 Embo J 10:1629; Gauss et al., 1992 Nucleic Acids Res 20:6739; Gauss et al., 1992 Genes Dev 6:1553; Gauss et al., 1993 Mol Cell Biol 13:3900; Gerstein et al., 1993 Genes Dev 7:1459; Ezekiel et al., 1995 Immunity 2:381; Fabb et al., 1995 Mol Cell Biol Hum Dis Ser 5:104; Davies et al., 1996 Methods Mol Biol 54:281; Dul et al., 1996 J Immunol 157:2969; Eastman et al., 1996 Nature 380:85; Fanning et al., 1996 Immunogenetics 44:146; Gauss et al., 1996 Mol Cell Biol 16:258; Eastman et al., 1997 Nucleic Acids Res 25:4370; Ezekiel et al., 1997 Mol Cell Biol 17:4191; Delassus et al., 1998 J Immunol 160:3274; Frank et al., 1998 Nature 396:173; Gauss et al., 1998 Eur J Immunol 28:351; Grawunder et al., 1998 J Biol Chem 273:24708; Eastman et al., 1999 Mol Cell Biol 19:3788; Fugmann et al., 2000 Annu Rev Immunol 18:495; Gellert, 2002 Annu Rev Biochem 71:101; Dai et al., 2003 Proc Natl Acad Sci USA 100:2462; De et al., 2004 Mol Cell Biol 24:6850; Espinoza et al., 2005 J Immunol 175:6668; Drejer-Teel et al., 2007 Mol Cell Biol 27:6288; Horne et al., 1982 J Immunol 129:660; Hamel et al., 1987 J Immunol 139:3012; Hesse et al., 1987 Cell 49:775; Hoeijmakers et al., 1987 Exp Cell Res 169:111; Koiwai et al., 1987 Biochem Biophys Res Commun 144:185; Kojima et al., 1987 Biochem Biophys Res Commun 143:716; Ichihara et al., 1988 Embo J 7:4141; Hesse et al., 1989 Genes Dev 3:1053; Hendrickson et al., 1991 Proc Natl Acad Sci USA 88:4061; Huang et al., 1992 J Clin Invest 89:1331; Ichihara et al., 1992 Immunol Lett 33:277; Kim, U. J. et al., 1992 Nucleic Acids Res 20:1083; Jakobovits et al., 1993 Nature 362:255; Knarr et al., 1995 J Biol Chem 270:27589; Huxley, 1997 Trends Genet 13:345; Julicher et al., 1997 Genomics 43:95; Hikida et al., 1998 J Exp Med 187:795; Ikeno et al., 1998 Nat Biotechnol 16:431; Kim, S. Y. et al., 1998 Genome Res 8:404; Hesslein et al., 2001 Adv Immunol 78:169; Holowka et al., 2001 Semin Immunol 13:99; Kaczmarczyk et al., 2001 Nucleic Acids Res 29:E56; Jones et al., 2003 Proc Natl Acad Sci USA 100:15446; Jung et al., 2003 Immunity 18:65; Kondo et al., 2003 Nucleic Acids Res 31:e76; Harder, 2004 Curr Opin Immunol 16:353; Ko et al., 2004 J Biol Chem 279:7715; Hayashi et al., 2005 Life Sci 77:1612; Ivanov et al., 2005 J Immunol 174:7773; Kapitonov et al., 2005 PLoS Biol 3:e181; Heaney et al., 2006 Mamm Genome 17:791; Inlay et al., 2006 J Exp Med 203:1721; Jung et al., 2006 Annu Rev Immunol 24:541; Heckmann et al., 2007 Methods Enzymol 426:463; Hillion et al., 2007 J Immunol 179:6790; Hillion et al., 2007 Autoimmun Rev 6:415; Meyerowitz et al., 1980 Gene 11:271; Landau et al., 1987 Mol Cell Biol 7:3237; Lee et al., 1999 Immunity 11:771; Lieber et al., 1987 Genes Dev 1:751; McCormick et al., 1987 Methods Enzymol 151:397; Lieber et al., 1988 Cell 55:7; Lieber et al., 1988 Proc Natl Acad Sci USA 85:8588; Lewis, 1994 Proc Natl Acad Sci USA 91:1332; Lieber et al., 1994 Semin Immunol 6:143; Lonberg et al., 1994 Nature 368:856; Lilie et al., 1995 J Mol Biol 248:190; Lonberg et al., 1995 Int Rev Immunol 13:65; Mattila et al., 1995 Eur J Immunol 25:2578; Livak et al., 1996 Mol Cell Biol 16:609; Leu et al., 1997 Immunity 7:303; Livak et al., 1997 J Mol Biol 267:1; Larijani et al., 1999 Nucleic Acids Res 27:2304; Modesti et al., 1999 Embo J 18:2008; Maes et al., 2000 J Immunol 165:703; Moshous et al., 2000 Hum Mol Genet 9:583; Mageed et al., 2001 Clin Exp Immunol 123:1; Moshous et al., 2001 Cell 105:177; Larin et al., 2002 Trends Genet 18:313; Ma et al., 2002 Cell 108:781; Lee et al., 2003 PLoS Biol 1:E1; Market et al., 2003 PLoS Biol 1:E16; Martin et al., 2003 J Immunol 171:4663; Montalbano et al., 2003 J Immunol 171:5296; Morshead et al., 2003 Proc Natl Acad Sci USA 100:11577; Moshous et al., 2003 Ann N Y Acad Sci 987:150; Le Deist et al., 2004 Immunol Rev 200:142; Li et al., 2005 J Immunol 174:2420; London, 2005 Biochim Biophys Acta 1746:203; Maes et al., 2006 J Immunol 176:5409; Masuda et al., 2006 Febs J 273:2184; Masumoto et al., 2006 Tanpakushitsu Kakusan Koso 51:2155; Monaco et al., 2006 Biochem Soc Trans 34:324; Lu et al., 2007 Nucleic Acids Res 35:6917; Lantelme et al., 2008 Mol Immunol 45:328; Ravetch et al., 1981 Cell 27:583; Peterson et al., 1984 Proc Natl Acad Sci USA 81:4363; Reth, M. G. et al., 1985 Nature 317:353; Rinfret et al., 1985 J Immunol 135:2574; Padlan et al., 1986 Mol Immunol 23:951; Reth, M. G. et al., 1986 Embo J 5:2131; Reth, M. et al., 1987 Embo J 6:3299; Pavan et al., 1990 Mol Cell Biol 10:4163; Ramsden et al., 1991 Proc Natl Acad Sci USA 88:10721; Rathbun et al., 1993 Int Immunol 5:997; Ramsay, 1994 Mol Biotechnol 1:181; Rolink et al., 1995 Semin Immunol 7:155; Pan et al., 1997 Int Immunol 9:515; Raaphorst et al., 1997 Int Immunol 9:1503; Roch et al., 1997 Nucleic Acids Res 25:2303; Nadel et al., 1998 J Exp Med 187:1495; Ohmori et al., 1998 Crit Rev Immunol 18:221; Ripoll et al., 1998 Gene 210:163; Nitschke et al., 2001 J Immunol 166:2540; Rooney et al., 2002 Mol Cell 10:1379; Oberdoerffer et al., 2003 Nucleic Acids Res 31:e140; Roose et al., 2003 PLoS Biol 1:E53; Poinsignon et al., 2004 J Exp Med 199:315; Repasky et al., 2004 J Immunol 172:5478; Reddy et al., 2006 Genes Dev 20:1575; Sandri-Goldin et al., 1981 Mol Cell Biol 1:743; Schatz et al., 1988 Cell 53:107; Schroeder et al., 1988 Proc Natl Acad Sci USA 85:8196; Sauer et al., 1990 New Biol 2:441; Yamada et al., 1991 J Exp Med 173:395; Schatz et al., 1992 Annu Rev Immunol 10:359; Seto et al., 1992 Nucleic Acids Res 20:3786; Solin et al., 1992 Immunogenetics 36:306; Taylor et al., 1992 Nucleic Acids Res 20:6287; Shapiro et al., 1993 Mol Cell Biol 13:5679; Tuaillon et al., 1993 Proc Natl Acad Sci USA 90:3720; Wei et al., 1993 J Biol Chem 268:3180; Schlissel et al., 1994 J Immunol 153:1645; Slightom et al., 1994 Gene 147:77; Woo et al., 1994 Nucleic Acids Res 22:4922; Schatz, 1997 Semin Immunol 9:149; Sauer, 1998 Methods 14:381; Skowronek et al., 1998 Proc Natl Acad Sci USA 95:1574; Tuaillon et al., 1998 Proc Natl Acad Sci USA 95:1703; Yu, C. C. et al., 1998 J Immunol 161:3444; Sun et al., 1999 Mol Immunol 36:551; Yu, K. et al., 1999 Mol Cell Biol 19:8094; Soderlind et al., 2000 Nat Biotechnol 18:852; Tevelev et al., 2000 J Biol Chem 275:8341; Tuaillon et al., 2000 J Immunol 164:6387; Tuaillon et al., 2000 Eur J Immunol 30:2998; Shizuya et al., 2001 Keio J Med 50:26; Wang et al., 2001 Genome Res 11:137; Williams et al., 2001 J Immunol 167:257; Sauer, 2002 Endocrine 19:221; Schlissel, 2002 Cell 109:1; Tsai et al., 2002 Genes Dev 16:1934; Verkaik et al., 2002 Eur J Immunol 32:701; Yu, Y. et al., 2003 DNA Repair (Amst) 2:1239; Yurchenko et al., 2003 Genes Dev 17:581; Schatz, 2004 Immunol Rev 200:5; Shockett et al., 2004 Mol Immunol 40:813; Souto-Carneiro et al., 2004 J Immunol 172:6790; That et al., 2004 J Immunol 173:4009; Wollscheid et al., 2004 Subcell Biochem 37:121; Schatz et al., 2005 Curr Top Microbiol Immunol 290:49; Schelonka et al., 2005 J Immunol 175:6624; Spicuglia et al., 2006 Curr Opin Immunol 18:158; Suarez et al., 2006 Mol Immunol 43:1827; Semprini et al., 2007 Nucleic Acids Res 35:1402; Takada et al., 2007 Genome Biol 8:215; VanDyk et al., 1996 J. Immunol 157: 4005-4015; Vanura et al., 2007 PLoS Biol 5:e43; Zheng et al., 2007 Mol Immunol 44:2221; Zou et al., 2007 Chin Med J (Engl) 120:410.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

We claim:

1. An isolated recombination-competent host cell comprising a nucleic acid composition for generating protein structural diversity comprising a tripartite recombination substrate, wherein the tripartite recombination substrate comprises:
(a) a first nucleic acid sequence operably linked to an expression control sequence and consisting essentially of (i) a first polynucleotide sequence that encodes at least a first portion of a protein, and (ii) a first recombination signal sequence located 3′ to the first polynucleotide sequence;
(b) a second nucleic acid sequence consisting essentially of (i) a second polynucleotide sequence that encodes at least a second portion of a protein, (ii) a second recombination signal sequence located 5′ to the second polynucleotide sequence that is capable of functional recombination with the first recombination signal sequence, and (iii) a third recombination signal sequence located 3′ to the second polynucleotide sequence; and
(c) a third nucleic acid sequence consisting essentially of (i) a third polynucleotide sequence that encodes at least a third portion of a protein, and (ii) a fourth recombination signal sequence located 5′ to the third polynucleotide sequence that is capable of functional recombination with the third recombination signal sequence,
wherein the tripartite recombination substrate can undergo recombination in the isolated host cell to form a recombined polynucleotide that encodes a structurally diversified protein, and wherein the isolated host cell expresses the structurally diversified protein, and wherein at least one of the first, second and third portions is a portion of a non-immunoglobulin protein.

2. The isolated host cell of claim 1, wherein the first, second and third portions are each a portion of a non-immunoglobulin protein.

3. The isolated host cell of claim 2, wherein the first, second and third portions are each a portion of the same non-immunoglobulin protein.

4. The isolated host cell of claim 1, wherein at least one of the first, second and third portions is a portion of an immunoglobulin protein.

5. The isolated host cell of claim 1, wherein the nucleic acid composition further comprises a fourth nucleic acid sequence that comprises a polynucleotide sequence encoding a membrane anchor domain operably linked to the tripartite recombination substrate, and wherein the expressed protein comprises a membrane anchor domain.

6. The isolated host cell of claim 5, wherein the membrane anchor domain polypeptide comprises a transmembrane domain peptide, a glycosylphosphatidylinositol-linkage polypeptide, a lipid raft-associating polypeptide, or a specific protein-protein association domain polypeptide.

7. The isolated host cell according to claim 1, wherein the nucleic acid composition is maintained extrachromosomally in the isolated host cell.

8. The isolated host cell according to claim 1, wherein the nucleic acid composition is integrated into the genome of the isolated host cell.

9. The isolated host cell according to claim 1, wherein the first, second and third nucleic acid sequences are joined in operable linkage as a single nucleic acid molecule.

10. The isolated host cell according to claim 1, wherein the first, second and third nucleic acid sequences are joined in operable linkage in a vector.

11. The isolated host cell according to claim 1, wherein the expression control sequence is selected from the group consisting of: a constitutive promoter, a regulated promoter, a repressor binding site and an activator binding site.

12. The isolated host cell according to claim 11, wherein the expression control sequence is an inducible promoter.

13. The isolated host cell according to claim 11, wherein the expression control sequence is a tightly regulated promoter.

14. The isolated host cell according to claim 1, wherein the isolated host cell is genetically engineered to express a mammalian RAG-1 gene, a mammalian RAG-2 gene and a mammalian TdT gene, or a fragment thereof that encodes a protein that is capable of mediating gene rearrangement and junctional diversity.

15. A method for generating structural diversity in a protein comprising maintaining the isolated host cell of claim 1 under conditions and for a time sufficient to allow for recombination of the tripartite recombination substrate and expression of the recombined polynucleotide, thereby generating a structurally diversified protein.

16. The method of claim 15, wherein the first, second and third portions are each a portion of a non-immunoglobulin protein.

17. The method of claim 15, wherein the first, second and third portions are each a portion of the same non-immunoglobulin protein.

18. The method of claim 15, wherein at least one of the first, second and third portions is a portion of an immunoglobulin protein.

19. The method according to claim 15, wherein the nucleic acid composition further comprises a fourth nucleic acid sequence that comprises a polynucleotide sequence encoding a membrane anchor domain operably linked to the tripartite recombination substrate, and the recombination events result in formation of a recombined polynucleotide that encodes a protein having a membrane anchor domain.

20. The method according to claim 15, wherein the step of maintaining the isolated host cell comprises maintaining under conditions and for a time sufficient for expression of the non-immunoglobulin protein.

21. The method according to claim 15, further comprising, prior to the step of maintaining, expanding the isolated host cell to obtain a plurality of recombination-competent host cells each comprising at least one tripartite recombination substrate.

22. The method according to claim 15, wherein the nucleic acid composition is maintained extrachromosomally in the isolated host cell.

23. The method according to claim 15, wherein the nucleic acid composition is integrated into the genome of the isolated host cell.

24. The method according to claim 15, wherein the first, second and third nucleic acid sequences are joined in operable linkage as a single nucleic acid molecule.

25. The method according to claim 15, wherein the first, second and third nucleic acid sequences are joined in operable linkage in a vector.

26. The method according to claim 15, wherein the expression control sequence is selected from the group consisting of: a constitutive promoter, a regulated promoter, a repressor binding site and an activator binding site.

27. The method according to claim 26, wherein the expression control sequence is an inducible promoter.

28. The method according to claim 26, wherein the expression control sequence is a tightly regulated promoter.

29. The method according to claim 15, wherein the isolated host cell is genetically engineered to express a mammalian RAG-1 gene, a mammalian RAG-2 gene and a mammalian TdT gene, or a fragment thereof that encodes a protein that is capable of mediating gene rearrangement and junctional diversity.

30. The method according to claim 18, wherein the tripartite recombination substrate is under control of an inducible recombination control element, and wherein the step of maintaining comprises contacting the plurality of isolated host cells with a recombination inducer.

31. The method according to claim 15, wherein the isolated recombination-competent host cell is selected from the group consisting of: (a) an isolated host cell that is capable of dividing without recombination occurring; (b) an isolated host cell that can be induced to express one or more recombination control elements selected from a RAG-1 gene and a RAG-2 gene; and (c) an isolated host cell that expresses first and second recombination control elements that comprise, respectively, a RAG-1 gene, and a RAG-2 gene, wherein expression of at least one of said recombination control elements by the host cell can be substantially impaired.
